(19) United States
(12) Patent Application Publication
(10) Pub. No.: US 2024/0296903 A1
(43) Pub. Date: Sep. 5, 2024

(54) METHODS OF PLACING LOCKED NUCLEIC ACIDS IN SMALL INTERFERING RNA STRANDS

(71) Applicant: Switch Therapeutics Inc., South San Francisco, CA (US)

(72) Inventor: Si-ping Han, South San Francisco, CA (US)

(21) Appl. No.: 18/575,654

(22) PCT Filed: Jul. 5, 2022
(86) PCT No.: PCT/US2022/073432

§ 371 Date: Dec. 29, 2023

Related U.S. Application Data
(60) Provisional application No. 63/218,865, filed on Jul. 6, 2021.

(51) Int. Cl. G16B 15/10; C12N 15/113

(52) U.S. Cl. CPC G16B 15/10; C12N 15/113; C12N 2310/11; C12N 2310/322; C12N 2310/3231

(57) ABSTRACT

Provided herein include methods, systems, and compositions for placing locked nucleic acids in small interfering RNA (siRNA) strands, for example conditionally activatable siRNA sensor strands, as well as the siRNA complexes generated using the method herein described and the component strands. The siRNA complex can be conditionally activated upon a complementary binding to an input nucleic acid strand (e.g. a mRNA of a biomarker gene specific to a target cell) through a sequence in a sensor nucleic acid strand of the nucleic acid complex. The activated nucleic acid complex can release a RNAi duplex which can specifically inhibit a target RNA.

### CROSS-REFERENCE TO RELATED APPLICATIONS

This application claims the benefit under 35 U.S.C. § 119(e) to U.S. Provisional Patent Application No. 63/218,865 filed on Jul. 6, 2021, the content of which is incorporated herein by reference in its entirety for all purposes.

### REFERENCE TO SEQUENCE LISTING

The present application is being filed along with a Sequence Listing in electronic format. The Sequence Listing is provided as a file entitled 75EN-329796-WO, created Jul. 4, 2022, which is 238 kilobytes in size. The information in the electronic format of the Sequence Listing is incorporated herein by reference in its entirety.

### BACKGROUND

### Field

The present disclosure relates generally to the field of nucleic acid, for example, small interfering RNA complexes.

### Description of the Related Art

Despite emerging developments in the field of dynamic nuclei acid nanotechnology and biomolecular computing, there is still a challenge to develop targeted RNAi therapy that can use nuclei acid logic switches to sense RNA transcripts (such as mRNAs and miRNAs), thereby restricting RNA interfering (RNAi) therapy to specific populations of disease-related cells. In particular, there is a need to develop targeted and conditionally activated RNAi therapy with improved drug potency, sensitivity, and stability, low design complexity, and low dosage requirement.

### SUMMARY

Disclosed herein includes a method of introducing a plurality of locked nucleic acid (LNA) modifications in a nucleic acid strand, comprising: under control of a hardware processor: (a) determining a secondary structure of the nucleic acid strand; (b) determining if each nucleotide of the nucleic acid strand is capable of base pairing with another nucleotide of the nucleic acid strand; (c) identifying a plurality of sequence regions in the nucleic acid strand, each having a starting position and an end position and each comprising a nucleotide capable of base pairing with at most one other nucleotide in the secondary structure; (d) for each of the plurality of sequence region identified in (c), from the starting position to the end position (i) introducing a first LNA modification at a first nucleotide of the sequence region capable of forming a minimum number of base pairs with other nucleotides of the nucleic acid strand; (ii) introducing a second LNA modification at a second nucleotide of the sequence region at least two bases downstream from the first LNA modification introduced if the second nucleotide is not a guanine (G); and (iii) repeating (ii) until reaching the end position of the sequence region to generate a LNA-modified nucleic acid strand.

The secondary structure of the nucleic acid strand can comprise an internal secondary structure formed by the nucleic acid strand, a self-duplex secondary structure formed by two interacting nucleic acid strands, or both. In some embodiments, the secondary structure of the nucleic acid strand has a minimal free energy. In some embodiments, obtaining the secondary structure of the nucleic acid strand comprises calculating a free energy of each of a plurality of secondary structures of the nucleic acid strand and identifying the secondary structure of the nucleic acid strand having a minimal free energy. In some embodiments, calculating the free energy of each of the plurality of secondary structures of the nucleic acid strand comprises using a nearest neighbor model. In some embodiments, analyzing the secondary structure of the nucleic acid strand comprises: determining a base pair score for each nucleotide of the nucleic acid strand, the base pair score being proportional to the number of base pairs a nucleotide forms with one or more nucleotides of the nucleic acid strand. In some embodiments, the base pairs are formed between nucleotides of a single nucleotide acid strand. In some embodiments, the base pairs are formed between nucleotides of two interacting nucleotide acid strands. In some embodiments, the number of base pairs the nucleotide forms with one or more nucleotides of the nucleic acid strand is at most three. In some embodiments, identifying the plurality of sequence regions each having a starting position and an end position and each comprising a nucleotide forming at most one base pair with another nucleotide in the secondary structure, comprises: identifying, for each sequence region, at least one nucleotide having a lowest base pair score.

The method can comprise eliminating any sequence region in which each nucleotide of the sequence region is capable of base pairing with two or more other nucleotides in the nucleic acid strand. In some embodiments, the plurality of sequence regions each have 2-20 nucleotides in length. In some embodiments, the first nucleotide of the sequence region at which the first LNA modification is introduced is not a G.

In some embodiments, the method comprises: identifying one or more nucleotide of the sequence region each forming a minimum number of base pairs with other nucleotides of the nucleic acid strand. In some embodiments, the second nucleotide of the sequence region forms a minimum number of base pairs with other nucleotides of the nucleic acid strand. In some embodiments, introducing the first LNA modification at the first nucleotide of the sequence region forming the minimum number of base pairs with other nucleotides of the nucleic acid strand comprises: identifying the first nucleotide of the sequence region that is not guanine (G) and that forms the minimum number of base pairs with other nucleotides of the nucleic acid strand; and introducing the first LNA modification at the first nucleotide of the sequence region. In some embodiments, the method comprises: introducing the first LNA modification at the first nucleotide of the sequence region that is a G and that form a minimum number of base pairs with other nucleotides of the nucleic acid strand, if all the nucleotides of the sequence region each forming the minimum number of base pairs with other nucleotides of the nucleic acid strand are G.

In some embodiments, the second LNA modification is introduced at the second nucleotide of the sequence region three bases downstream from the first LNA modification introduced. In some embodiments, the second LNA modification is introduced at the second nucleotide of the sequence region four bases downstream from the first LNA modification introduced. In some embodiments, the plurality of sequence regions does not overlap with one another when aligned with the nucleic acid strand. In some embodiments, the LNA modification comprises introducing a chemical bridge connecting the 2′ and 4′ carbons of a nucleotide. The chemical bridge can be, for example, a 2′-O, 4′-C methylene bridge or a 2′-O, 4′-C ethylene bridge. In some embodiments, about 10%-50% of the nucleotides of the nucleic acid strand are modified with LNA or analogues thereof. In some embodiments, any two LNA modifications of the plurality of LNA modifications are at least one nucleotide apart. In some embodiments, two LNA modifications of the plurality of LNA modifications are two nucleotides apart. In some embodiments, the 5′ terminus, the 3′ terminus, or both of the LNA-modified nucleic acid strand comprises a terminal moiety. The terminal moiety can, for example, comprise a ligand, a fluorophore, a exonuclease, a fatty acid, a Cy3, an inverted dT attached to a tri-ethylene glycol, or a combination thereof. In some embodiments, the nucleotide acid strand has 10-35 nucleotides in length. In some embodiments, the method further comprises producing the LNA-modified nucleic acid strand.

Provided herein includes a method for producing a nucleic acid complex, comprising: providing a first nucleic acid strand comprising 20-70 linked nucleosides; providing a second nucleic acid strand; providing a third nucleic acid strand, wherein one or more of the first, second and third nucleic acid strands is a LNA-modified nucleic acid strand produced by any of the methods disclosed herein; and contacting the first nucleic acid strand, the second nucleic strand, and the third nucleic acid strand under a condition for a period of time to form a nucleic acid complex, wherein the nucleic acid complex comprises: the second nucleic acid strand binding to a central region of the first nucleic acid strand to form a first nucleic acid duplex; and the third nucleic acid strand binding to a 5′ region and a 3′ region of the first nucleic acid strand to form a second nucleic acid duplex, wherein the third nucleic acid strand comprises a 3′ toehold that is not complementary to the first nucleic acid strand and is capable of binding to an input nucleic acid strand to cause the displacement of the third nucleic acid strand from the first nucleic acid strand. The central region of the first nucleic acid strand can comprise a sequence complementary to a target RNA. In some embodiments, the sequence complementary to a target RNA is 10-35 nucleosides in length.

Also disclosed herein include a method for producing a nucleic acid complex, comprising: providing a first nucleic acid strand comprising 20-70 linked nucleosides; providing a second nucleic acid strand; providing a third nucleic acid strand, wherein one or more of the first, second and third nucleic acid strands is a LNA-modified nucleic acid strand produced by any of the methods disclosed herein; and contacting the first nucleic acid strand, the second nucleic strand, and the third nucleic acid strand under a condition for a period of time to form a nucleic acid complex, wherein the nucleic acid complex comprises: the second nucleic acid strand binding to a first region of the first nucleic acid strand to form a first nucleic acid duplex; and the third nucleic acid strand binding to a second region of the first nucleic acid strand to form a second nucleic acid duplex, wherein the third nucleic acid strand comprises a 3′ toehold that is not complementary to the first nucleic acid strand and is capable of binding to an input nucleic acid strand to cause the displacement of the third nucleic acid strand from the first nucleic acid strand, and wherein the first region of the first nucleic acid strand is 3′ of the second region of the first nucleic acid strand, and the third nucleic acid strand does not bind to any region of the first nucleic acid strand that is 3′ of the first region of the first nucleic acid strand. In some embodiments, the first region of the first nucleic acid strand comprises a sequence complementary to a target RNA. The sequence complementary to a target RNA can be, for example, 10-35 nucleosides in length. In some embodiments, the third nucleic acid strand comprises a 5′ toehold

Details of one or more implementations of the subject matter described in this specification are set forth in the accompanying drawings and the description below. Other features, aspects, and advantages will become apparent from the description, the drawings, and the claims. Neither this summary nor the following detailed description purports to define or limit the scope of the inventive subject matter.

### BRIEF DESCRIPTION OF THE DRAWINGS

FIG. 1 is a flow diagram showing an exemplary method of placing locked nucleic acids (LNAs) in a nucleic acid strand, e.g., a small interfering RNA (siRNA) strand.

FIG. 2 is a block diagram of an illustrative computing system configured to place LNAs in a siRNA strand.

FIG. 3 is a flow diagram showing a non-limiting workflow for placing LNAs in the sensor strand of a conditionally activatable siRNA complex.

FIG. 4 illustrates a schematic representation of two non-limiting exemplary nucleic acid complex constructs.

FIG. 5 illustrates a schematic representation of a non-limiting exemplary nucleic acid complex construct.

FIG. 6 illustrates a schematic representation of two non-limiting exemplary nucleic acid complex constructs.

FIG. 7 illustrates a schematic representation of a sensor nucleic acid strand, a core nucleic acid strand and a passenger nucleic acid strand of a non-limiting exemplary nucleic acid complex.

FIG. 8 illustrates a schematic representation of a non-limiting exemplary nucleic acid complex construct with regions for screening highlighted in yellow.

FIG. 9 is a non-limiting schematic diagram showing the formation of an active RNAi duplex following the displacement of a sensor nucleic acid strand from a core nucleic acid strand and the degradation of the core nucleic acid strand overhangs.

FIG. 10A and FIG. 10B show sequence diagrams of two non-limiting exemplary nucleic acid complex constructs having the same passenger strand but different core strand. Core strand v3c1: from 5′ to 3′ SEQ ID NO: 3-5 joined by a C3 spacer; Passenger strand v3p1: SEQ ID NO: 2; Core strand v3c5: SEQ ID NO: 11; Passenger strand 1: SEQ ID NO: 2.

FIG. 11 show sequence diagrams of two positive control constructs. HTT Guide 1: SEQ ID NO: 21; HTT Pass 1: SEQ ID NO: 22; HTT Guide 2: SEQ ID NO: 23; HTT Pass 2: SEQ ID NO: 24.

FIG. 12 shows various siRNA complex variants with different passenger strand (V3P1, V3P2, V3P3, V3P5, V3P5, V3P6, V3P7, V3P8, and V3P9) assembled with an exemplary core strand (v3c1 which include two C3 linkers) shown in FIG. 10A and used in target protein expression shown in FIG. 13.

FIG. 13 shows a graphic representation of the target protein expression data generated using the siRNA complex deign variants shown in FIG. 12.

FIG. 14 shows various siRNA complex variants with different passenger strand (V3P1, V3P2, V3P3, V3P5, V3P5, V3P6, V3P7, V3P8, and V3P9) assembled with an exemplary core strand (v3c5 which does not include a C3 linker) shown in FIG. 10B.

FIG. 15 shows a graphic representation of the target protein expression data generated using the siRNA complex variants shown in FIG. 14.

FIG. 16A and FIG. 16B show sequence diagrams of various exemplary nucleic acid complex constructs each having the same passenger strand (Passenger strand 1) and the same sensor strand (Mir23 Sensor 1) but a different core strand (Core strand v3c1, Core strand v3c2, Core strand v3c3, Core strand v3c4, Core strand v3c5, and Core strand v3c6, which are referred to as C1, C2, C3, C4, C5, C6, respectively, in FIGS. 18-19 and description thereof). The sequences shown in FIGS. 16A and 16B are listed in Table 2.

FIG. 17 shows non-denaturing polyacrylamide gel (PAGE) of various nucleic acid complex constructs.

FIG. 18 shows the RNAi activity of two-stranded assemblies each having the same passenger strand v3p1 and a different core strand (C1, C2, C3, C4, C5, and C6) at different concentrations.

FIG. 19 shows the RNAi activity of three-stranded assemblies each having the same passenger strand v3p1, the same sensor strand (Mir23 sensor 1), and a different core strand (C1, C2, C3, C4, C5, and C6) at three different concentrations.

FIG. 20 shows sequence diagrams of a non-limiting exemplary nucleic acid complex construct disclosed herein (top: V3C3a) and a partially modified nucleic acid complex (bottom: G1C1S1). The sequences shown in FIG. 20 are listed in Table 3.

FIG. 21 shows the RNAi activity of the exemplary two-stranded nucleic acid complex constructs (V3C3a siRNA) and three-stranded nucleic acid complex constructs (V3C3a and V3C3b) in comparison with the partially modified two-stranded construct (G1C1 siRNA) and the partially modified three-stranded constructs (G1C1S1) shown in FIG. 20 at three different concentrations.

FIG. 22 shows sequence diagrams of three non-limiting exemplary nucleic acid complex constructs. Alt anp sens1: SEQ ID NO: 33; Alt anp-calc core 1: SEQ ID NO: 34; Alt anp sens2: SEQ ID NO: 35; Alt mus-calc core2: SEQ ID NO: 36; Alt mus-calc core 3: SEQ ID NO: 37. Calc V3P3 passenger: SEQ ID NO: 13.

Throughout the drawings, reference numbers may be re-used to indicate correspondence between referenced elements. The drawings are provided to illustrate example embodiments described herein and are not intended to limit the scope of the disclosure.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

RNA interference (RNAi) is an intrinsic cellular mechanism conserved in most eukaryotes, that helps to regulate the expression of genes critical to cell fate determination, differentiation, survival and defense from viral infection. Researchers have exploited this natural mechanism by designing synthetic double-stranded RNA for sequence-specific gene silencing. Emerging developments in the field of dynamic nuclei acid nanotechnology and biomolecular computing also offer a conceptual approach to design programmable RNAi agents. However, challenges still remain in developing targeted RNAi therapy that can use nuclei acid logic switches to sense RNA transcripts (such as mRNAs and miRNAs) in order to restrict RNA silencing to specific populations of disease-related cells and spare normal tissues from toxic side effects. Significant challenges include poorly suppressed background drug activity, weak activated state drug potency, input and output sequence overlap, high design complexity, short lifetimes (<24 hours) and high required device concentrations (>10 nM).

Provided herein includes a method for placing locked nucleic acids (LNAs) in conditionally activable small interfering RNA (siRNA) strands (e.g. a sensor nucleic acid strand or a segment thereof). Provided herein also includes the nucleic acid complex generated using the method herein described as well as the component strands of the nucleic acid complex (e.g. the core nucleic acid strand, the LNA-modified sensor nucleic acid strand, and the passenger nucleic acid strand). The conditionally activatable siRNA complex generated using the method herein described can switch from an inactivated state to an activated state when triggered by a complementary binding of an input nucleic acid strand (e.g. a disease biomarker gene specific to disease-related cells) to the siRNA complex, thereby activating the RNA interference activity of the siRNA complex to target a specific target RNA (e.g. a RNA to be silenced). The nucleic acid complexes herein described can mediate conditionally activated RNA interference activity to silence target RNA in specific populations of disease-related cells with improved potency at a low concentration as well as improved specificity that can reduce off-target effects.

Disclosed herein includes a method for introducing a plurality of LNA modification in a nucleic acid strand. The method can comprise under control of a hardware processor, obtaining a secondary structure of the nucleic acid strand. The method can comprise determining if each nucleotide of the nucleic acid strand is capable of base pairing with another nucleotide of the nucleic acid strand. The method can comprise identifying a plurality of sequence regions in the nucleic acid strand, each having a starting position and an end position and each comprising a nucleotide capable of base pairing with at most one other nucleotide in the secondary structure. For each of the plurality of sequence regions identified, from the starting position to the end position, the method introduces a first LNA modification at a first nucleotide of the sequence region capable of forming a minimum number of base pairs with other nucleotides of the nucleic acid strand. The method can comprise introducing a second LNA modification at a second nucleotide of the sequence region at least two bases downstream from the first LNA modification introduced if the second nucleotide is not a guanine (G). The method can also comprise repeating the processing by introducing one or more LNA modifications in the sequence region until reaching the end position of the sequence region to provide a LNA-modified nucleic acid strand.

Disclosed herein also include a method for producing a nucleic acid complex. The method can comprise providing a first nucleic acid strand (e.g. a core nucleic acid strand), a second nucleic acid strand (e.g. a passenger nucleic acid strand), and a third nucleic acid strand (e.g. a sensor nucleic acid strand) and contacting the first, second and third nucleic acid strands under a condition for a period of time to form a nucleic acid complex. The core nucleic acid strand can comprise 20-70 linked nucleosides, optionally 20-60 linked nucleosides. One or more of the core nucleic acid strand, the passenger nucleic acid strand, and the sensor nucleic acid strand is a LNA-modified nucleic acid strand produced using the method described herein. In some embodiments, the formed nucleic acid complex can comprise the passenger nucleic acid strand binding to a central region of the core nucleic acid strand to form a RNAi duplex and the sensor nucleic acid strand binding to a 5′ region and a 3′ region of the core nucleic acid strand to form a sensor duplex. In some embodiments, the formed nucleic acid complex can comprise the passenger nucleic acid strand binding to a first region of the core nucleic acid strand to form a RNAi duplex and the sensor nucleic acid strand binding to a second region of the core nucleic acid strand to form a sensor duplex. The first region of the core nucleic acid strand is 3′ of the second region of the core nucleic acid strand. The sensor nucleic acid strand does not bind to any region of the core nucleic acid strand that is 3′ of the first region of the core nucleic acid strand. In some embodiments, the sensor nucleic acid strand comprises a 3′ toehold that is not complementary to the core nucleic acid strand and is capable of binding to an input nucleic acid strand to cause the displacement of the sensor nucleic acid strand from the core nucleic acid strand. In some embodiments, the sensor nucleic acid strand further comprises a 5′ toehold.

### Definitions

Unless defined otherwise, technical and scientific terms used herein have the same meaning as commonly understood by one of ordinary skill in the art to which the present disclosure belongs. See, e.g., Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, N Y 1989). For purposes of the present disclosure, the following terms are defined below.

As used herein, the term “nucleoside” refers to a molecule having a purine or pyrimidine base covalently linked to a ribose or deoxyribose sugar. Exemplary nucleosides include adenosine, guanosine, cytidine, uridine and thymidine.

The term “nucleotide” refers to a nucleoside having one or more phosphate groups joined in ester linkages to the sugar moiety. Exemplary nucleotides include nucleoside monophosphates, diphosphates and triphosphates.

The terms “polynucleotide” and “nucleic acid molecule” are used interchangeably herein and refer to a polymer of nucleotides joined together by a phosphodiester linkage between 5′ and 3′ carbon atoms.

The term “RNA” or “RNA molecule” or “ribonucleic acid molecule” refers to a polymer of ribonucleotides. The term “DNA” or “DNA molecule” or deoxyribonucleic acid molecule” refers to a polymer of deoxyribonucleotides. DNA and RNA can be synthesized naturally (e.g., by DNA replication or transcription of DNA, respectively). RNA can be post-transcriptionally modified. DNA and RNA can also be chemically synthesized. DNA and RNA can be single-stranded or multi-stranded (e.g., double-stranded or triple-stranded). “mRNA” or “messenger RNA” is single-stranded RNA molecule that is complementary to one of the DNA strands of a gene. “miRNA” or “microRNA” is a small single-stranded non-coding RNA molecule that functions in RNA silencing and post-transcriptional regulation of gene expression.

The term “RNA analog” refers to an polynucleotide having at least one altered or modified nucleotide as compared to a corresponding unaltered or unmodified RNA. The nucleotide can retain the same or similar nature or function as the corresponding unaltered or unmodified RNA such as forming base pairs.

A single-stranded polynucleotide has a 5′ terminus or 5′ end and a 3′ terminus or 3′ end The terms “5′ end” “5′ terminus” and “3′ end” “3′ terminus” of a single-stranded polynucleotide indicate the terminal residues of the single-stranded polynucleotide and are distinguished based on the nature of the free group on each extremity. The 5′-terminus of a single-stranded polynucleotide designates the terminal residue of the single-stranded polynucleotide that has the fifth carbon in the sugar-ring of the deoxyribose or ribose at its terminus (5′ terminus). The 3′-terminus of a single-stranded polynucleotide designates the residue terminating at the hydroxyl group of the third carbon in the sugar-ring of the nucleotide or nucleoside at its terminus (3′ terminus). The 5′ terminus and 3′ terminus in various cases can be modified chemically or biologically e.g. by the addition of functional groups or other compounds as will be understood by the skilled person.

As used herein, the terms “complementary binding” and “bind complementarily” mean that two single strands are base paired to each other to form nucleic acid duplex or double-stranded nucleic acid. The term “base pair” as used herein indicates formation of hydrogen bonds between base pairs on opposite complementary polynucleotide strands or sequences following the Watson-Crick base pairing rule. For example, in the canonical Watson-Crick DNA base pairing, adenine (A) forms a base pair with thymine (T) and guanine (G) forms a base pair with cytosine (C). In RNA base paring, adenine (A) forms a base pair with uracil (U) and guanine (G) forms a base pair with cytosine (C). A certain percentage of mismatches between the two single strands are allowed as long as a stable double-stranded duplex can be formed. In some embodiments, the two strands that bind complementarily can have a mismatches can be, be about, be at most, or be at most about, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50%.

As used herein, the terms “RNA interference”, “RNA interfering”, and “RNAi” refer to a selective intracellular degradation of RNA. RNAi can occur in cells naturally to remove foreign RNAs (e.g., viral RNAs). Natural RNAi proceeds via fragments cleaved from free dsRNA which direct the degradative mechanism to other similar RNA sequences. RNAi can also be initiated non-naturally, for example, to silence the expression of target genes.

As used herein, the terms “small interfering RNA” and “siRNA” refer to an RNA or RNA analog capable of reducing or inhibiting expression of a gene or a target gene when the siRNA is activated in the same cell as the target gene. The siRNA used herein can comprise naturally occurring nucleic acid bases and/or chemically modified nucleic acid bases (RNA analogs).

### Method of Placing LNAs in Nucleic Acid Strands

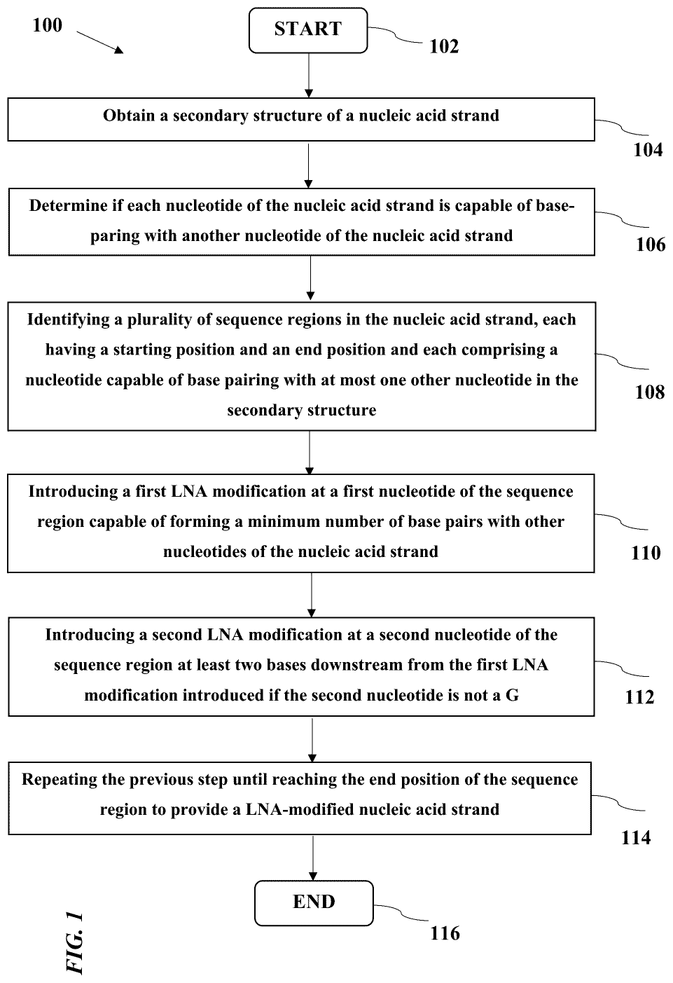

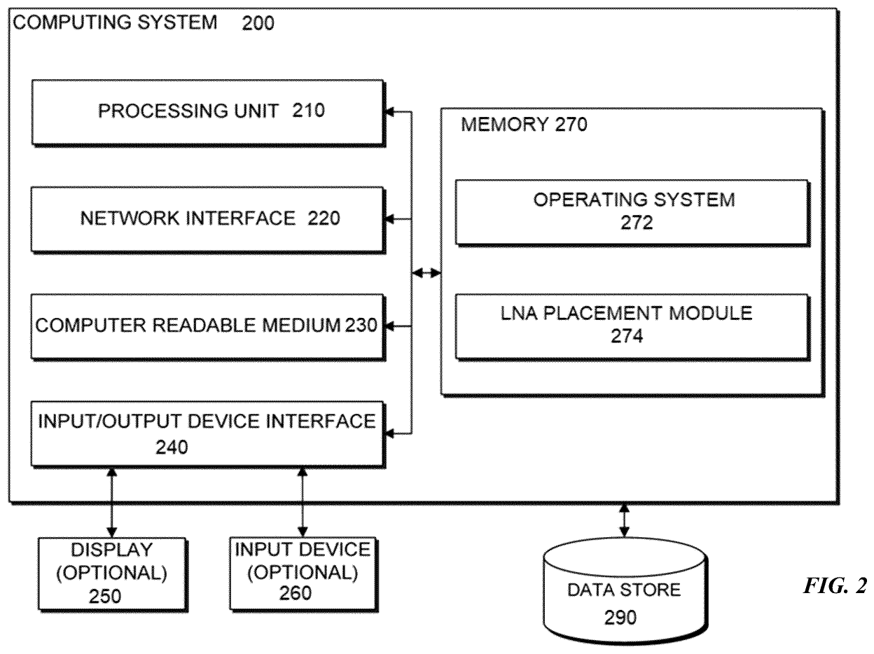

FIG. 1 is a flow diagram showing an exemplary method of 100 of placing LNAs in a nucleic acid strand (e.g. a sensor strand of a conditionally activatable siRNA complex) or a segment thereof. The method can be embodied in a set of executable program instructions stored on a computer-readable medium such as one or more disk drives, of a computing system. For example, the computing system 200 shown in FIG. 2 and described in greater details below can execute a set of executable program instructions to implement the method 100. When the method 100 is initiated, the executable program instructions can be loaded into memory, such as RAM, and executed by one or more processors of the computing system 200. Although the method 100 is described with respect to the computing system 200 shown in FIG. 2, the description is illustrative only and is not intended to be limiting. In some embodiments, the method 100 or portions thereof can be performed serially or in parallel by multiple systems.

After the method 100 begins at block 102, the method 100 proceeds to block 104, where a computing system (e.g. the computing system 200 shown in FIG. 2) can obtain a secondary structure of a nucleic acid strand (e.g. a sensor strand of a conditionally activatable siRNA complex) or a segment thereof to which the computing system will introduce LNAs. A secondary structure as used herein can comprise an internal secondary structure formed by base-pairing interaction within a single nucleic acid strand. For example, a RNA secondary structure can be composed of a stem structure formed by complementary pairing of contiguous bases and a cyclic structure by non-pairing of bases. A secondary structure can also comprise a self-duplex secondary structure formed by base-pairing interactions between two interacting nucleic acid molecules (e.g. two interacting sensor strands), such as a duplex.

In some embodiments, the computing system receives a sequence of a nucleic acid strand or a segment thereof, for example, from a user of the system and generates a secondary structure based on the received sequence. The sequence can be provided in any computer-readable format such as plain sequence format, FASTQ format, EMBL format, FASTA format, GenBank format or any other format identifiable to a person skilled in the art. The computing system can generate and/or cause to display a first user interface (UI). The first UI can comprise one or more input elements (e.g. one or more text boxes) for receiving the input sequence, information related to the input sequence, and parameters related to generating a secondary structure of the input sequence. The nucleic acid strand or a segment thereof can be 10-35 nucleotides in length. In some embodiments, the nucleic acid strand or a segment thereof is a sensor nucleic acid strand of a conditionally activatable siRNA complex described elsewhere herein or a segment thereof.

In some embodiments, generating a secondary structure of a RNA sequence comprises identifying a secondary structure (e.g. an internal secondary structure and/or a self-duplex secondary structure) of the RNA sequence having a minimal free energy (e.g. a minimal Gibbs free energy). In some embodiments, obtaining the secondary structure of the nucleic acid strand can comprise calculating a minimal free energy of an internal secondary structure formed by the nucleic acid strand. In some embodiments, obtaining the secondary structure of the nucleic acid strand can comprise calculating a minimal free energy of a self-duplex secondary structure formed by two interacting nucleic acid strands.

A secondary structure of a RNA molecule having a minimal free energy can also be referred to as an optimally folded structure. This can be carried out by predicting a plurality of folded secondary structures of the RNA molecule and calculating a free energy for each folded secondary structure. Accordingly, in some embodiments, the method can comprise calculating a free energy of each of a plurality of secondary structures of the nucleic acid strand and identifying the secondary structure of the nucleic acid strand having a minimal free energy (e.g. minimal Gibbs free energy).

In some embodiments, calculating a free energy of each of a plurality of secondary structures of the nucleic acid strand and identifying the secondary structure of the nucleic acid strand having a minimal free energy can be determined by thermodynamics.

For example, for a unimolecular reaction such as the folding of an RNA molecule (e.g. the complementary candidate sequence segment):

$$\begin{matrix}U \text{←→} F \\ K = [ F ] / [ U ] = {e}^{- Δ {G}^{0} / RT}\end{matrix}$$

wherein K is the equilibrium constant giving the ratio of concentration for folded, F, and unfolded, U, species at equilibrium. ΔG₀ is the standard free energy difference between F and U; R is the gas constant; and T is the temperature in kelvins. Secondary structure prediction involves identifying the base-pairing that gives the lowest free energy change in going from the unfolded to folded state and the highest concentration of folded species.

Exemplary computer programs for obtaining a secondary structure energy of RNA sequences include, but are not limited to, Mfold/UnaFold, the Vienna RNA package, RNAstructure, RNAsoft and Sfold. Table 1 provides a non-limiting list of the exemplary computer programs for RNA secondary structure prediction and free energy calculation and their URL.

TABLE 1
A non-limiting list of exemplary computer programs for RNA
secondary structure prediction and free energy calculation

| Program | URL |
|---|---|
| RNAstructure | rna.urmc.rochester.edu/RNAstructure.html |
| Sfold | sfold.wadsworth.org/ |
| UNAFold/Mfold | mfold.bioinfo.rpi.edu |
| Vienna RNA Package | www.tbi.univie.ac.at/RNA |
| RNAsoft | www.rnasoft.ca/ |

In some embodiments, calculating a minimal free energy of a secondary structure of a RNA sequence comprises using a dynamic programming to implicitly search the entire set of possible RNA secondary structures to find the lowest free energy structure without the necessity of generating all structures explicitly. For example, the free energy change can be typically approximated with a nearest neighbor model in which the ΔG₀ is the sum of free energy increments for the various nearest neighbor motifs (e.g. stacked base pairs in an RNA helix) that occur in a structure, as described in Turner 2000 (Conformational changes. In Nucleic acids (ed. Bloomfield V., Crothers D., Tinoco I. Jr), pp. 259-334 University Science Books, Sausalito, CA). Parameters for the nearest neighbor increments have been experimentally determined by various studies such as by optical melting studies, by relating parameters to the number of occurrences of various motifs in known secondary structures, by optimizing parameters to predict known secondary structures, or by a combination of these approaches as will be apparent to a skilled artisan. In some embodiments, calculating the free energy of each of the plurality of secondary structures of the nucleic acid strand comprises using a nearest neighbor model.

A nearest-neighbor method as used herein refers to a modeling algorithm for predicting RNA secondary structure. The algorithm is based on the approximation that the stabilities of a secondary structure depend on the sequences of each motif in the secondary structure and the interaction between neighboring base pairs. The overall stability of the secondary structure is the sum of individual stability increments for each motif. The nearest-neighbor model treats a nucleic acid helix as a string of interactions between ‘neighboring’ base pairs. For example, the nucleotide acid shown below has nearest-neighbor interactions indicated by the arrows.

↓ ↓ ↓ ↓ ↓
5′ C-G-U-U-G-A 3′
3′ G-C-A-A-C-U 5′

The free energy of forming the duplex above from the two individual strands, ΔG°, can be represented (e.g. at 37° C.) as

ΔG°₃₇(predicted)=ΔG°₃₇(C/G initiation)+ΔG°₃₇(CG/GC)+ΔG°₃₇(GU/CA)+ΔG°₃₇(UU/AA)+ΔG°₃₇(UG/AC)+ΔG°₃₇(GA/CU)+ΔG°₃₇(A/U initiation)

The first term represents the free energy of the first base pair, CG, in the absence of a nearest neighbor. The second term includes both the free energy of formation of the second base pair, GC, and stacking interaction between this base pair and the previous base pair. The remaining terms are similarly defined.

Each ΔG° term can be derived from enthalpy (ΔH°) and entropy changes (ΔS°):

ΔG°=ΔH°−TΔS°

The nearest neighbor parameters comprise the values of enthalpy changes (ΔH°) and entropy changes (ΔS°) for the ten possible pairs of interactions: AA/UU, AT/UA, UA/AU, CA/GU, GU/CA, CU/GA, GA/CU, CG/GC, GC/CG, GG/CC, as well as for the terminal A/U and G/C base pairs (e.g. initiation base pairs).

The nearest neighbor parameters herein described are temperature dependent and the nearest neighbor parameters can comprise parameters for folding a RNA at 37° C. (310.15 K). In some embodiments, the nearest neighbor parameters can be extrapolated to temperatures close to 37° C., in an approximate range of 10° C. to 60° C.

Nearest neighbor parameter sets can be divided into rules for individual motifs, which are helices or loops. Helices are composed of canonical base pairs (e.g. AU, GC, and GU). Loops are composed nucleotides of nucleotides not in canonical pairs and of junctions of helices. Descriptions of various individual motifs in a RNA secondary structure are available, for example, in rna.urmc.rochester.edu/NNDB/help.html, the content of which is incorporated by reference in its entirety.

Accordingly, in some embodiments, calculating the secondary structure (e.g. the internal secondary structure or the self-duplex secondary structure) of a nucleic acid strand uses nearest neighbor energy parameters.

Examples of nearest-neighbor parameters that can be used in the method herein disclosed are described, for example, in the Nearest Neighbor Database (rna.urmc.rochester.edu/NNDB/index.html) which is a web-based resource for disseminating parameter sets for predicting nucleic acid secondary structure stabilities. For each set of parameters, the database includes the set of rules with descriptive text, sequence-dependent parameters in plain text and html, literature references to experiments and usage tutorials. Nearest neighbor methods and parameters are also described, for example, in Turner 2010 (NNDB: the nearest neighbor parameter database for predicting stability of nucleic acid secondary structure. Nucleic Acids Res. 2010 January; 38:D280-2), the content of which is incorporated herein by reference. The nearest neighbor model and the nearest neighbor parameters can be implemented in a computer program that predicts low free energy secondary structures or optimally folded structures.

In some embodiments, the computing system can be connected to a public RNA secondary structure generation server indirectly via a wireless network connection. In some embodiments, the computing system can include a RNA secondary structure generation module that generates the secondary structure from an input sequence as described herein. The first UI of the computing system can comprise one or more input elements, such as one or more text boxes and/or one or more drop-down lists, for receiving parameters related to the secondary structure generation, such as nearest-neighbor parameters, temperature, maximum loop size, minimum helix length, maximum absolute energy difference (or maximum percent energy difference), and other parameters an user can further set for generating a secondary structure from sequence as will be apparent to a skilled person.

In some embodiments, the computing system receives a pre-generated secondary structure of a nucleic acid strand or a segment thereof. The secondary structure may be generated using any nucleic acid secondary structure prediction software known in the art such as the computer programs listed in Table 1.

The method 100 proceeds from block 104 to 106, where the computing system analyzes the secondary structure obtained in block 104 to determine if each nucleotide of the nucleic acid strand is capable of base-paring with another nucleotide of the nucleic acid strand.

In some embodiments, the secondary structure of the nucleic acid strand obtained in block 104 can be visually analyzed to determine if a nucleotide of the nucleic acid strand forms a base pair with another nucleotide of the nucleic acid strand and the number of other nucleotides each nucleotide of the nucleic acid strand can base pair with. The base pairs can be formed between nucleotides of a single nucleic acid strand, such as the base pairs formed in an internal secondary structure. The base pairs can also be formed between nucleotides of two interacting nucleic acid strands, such as in a duplex. Accordingly, a base pair used herein can refer to an intermolecular pair formed between two interacting nucleic acid strands (e.g. in a duplex), an intramolecular pair formed within a single nucleic acid strand, or both.

The secondary structure conformation of the nucleic acid strand can be represented in a graph or a diagram. Exemplary visualization approaches for characterizing the RNA secondary structure include, for example, planar graphs, linear art diagrams, circular diagrams and dot-plot diagrams as will be apparent to a person skilled in the art. For example, in a linear arc or circular diagram, the backbone can be represented by a straight line or a circle. The bases are consecutively placed along the line or the circle and paired bases are connected with arcs or chords. In a planar graph, the backbone is represented as a convoluted planar curves with predefined distances between neighboring paired bases and a minimum number of overlaps. Base pairing between the nucleotides of the nucleic acid strand can be characterized by examining the secondary structure diagrams or graphs and counting the number of base pairs each nucleotide is involved in.

In some embodiments, analyzing the secondary structure of the nucleic acid strand comprises determining a base-pairing score for each nucleotide of the nucleic acid strand. The base-pairing score of a nucleotide can be a numerical value proportional to the number of base pairs the nucleotide forms with one or more other nucleotides of the nucleic acid strand. The greater number of base pairs a nucleotide is involved in, the higher base-pairing score the nucleotide has. For example, a nucleotide may be assigned a numerical score of zero if the nucleotide is not involved in any base pair in the obtained internal secondary structure and/or self-duplex secondary structure. A nucleotide may be assigned a numerical score of one if the nucleotide is involved in one base pair or a numerical score of two if the nucleotide is involved in two base pairs. In some embodiments, the number of base pairs a nucleotide can form with other nucleotides of the nucleic acid strand is at most three (e.g. zero, one, two or three). In other words, a nucleotide can base pair with zero, one, two or three other nucleotides of the nucleic acid strand. The base pairs can be intramolecular base pairs, intermolecular base pairs, or both. In some embodiments, a nucleotide is not capable of base pairing with four or more other nucleotides of the nucleic acid strand.

The method 100 proceeds from block 106 to block 108, where the computing system identifies a plurality of sequence regions in the nucleic acid strand or a segment thereof. Each of the plurality of sequence regions is defined by a starting position and an end position. For example, the starting position of a sequence region can be the 5′ end of the sequence region and the end position of the sequence region can be the 3′ end of the sequence region. Each sequence region comprises a nucleotide capable of base pairing with at most one other nucleotide in the nucleotide acid strand, such as an unpaired nucleotide or a nucleotide forming a single base pair with another nucleotide. The plurality of sequence regions can be generated by segmenting the nucleic acid strand into multiple regions, each region having about 3-20 nucleotides in length. For example, each region of the plurality of sequence region generated from the nucleic acid strand can be 3, 4, 5, 6, 7, 8, 9, 10, 11, 12, 13, 14, 15, 16, 17, 18, 19, 20, or a range between any two of these values, nucleotides in length. In some embodiments, two of the sequence regions need not overlap with each other when aligned with the nucleic acid strand. In some embodiments, the plurality of sequence regions need not overlap with one another when aligned with the nucleic acid strand.

In some embodiments, identifying the plurality of sequence regions in the nucleic acid strand comprises eliminating any sequence region in which the least number of base pairs a nucleotide can form with other nucleotides of the nucleic acid strand in a secondary structure is two or more. In other words, any sequence region in which each nucleotide base pairs with two or more other nucleotides of the nucleic acid strand in the obtained internal and/or self-duplex secondary structure is eliminated.

In some embodiments, the method comprises, for each sequence region generated from the nucleic acid strand, identifying at least one nucleotide base pairing with the least number of other nucleotides. For example, the method can comprise, for each sequence region generated from the nucleic acid strand, assigning a base-pairing score for each nucleotide of the sequence region and identifying the nucleotide having the lowest base-pairing score in the sequence region. In some embodiments, more than one nucleotide may be associated with the lowest base-pairing score. The method can further comprise eliminating any sequence region having the lowest base-pairing score of the nucleotides greater than one (e.g., two or more).

The method 100 proceeds from block 108 to block 110, wherein, for each remaining sequence region, the computing system introduces a first LNA modification at a first nucleotide position of the sequence region capable of forming a minimum number of base pairs with other nucleotides of the nucleic acid strand.

In some embodiments, the method can scan the sequence region from the starting position (e.g. 5′ end) to the end position (e.g. 3′ end) to identify the first nucleotide of the sequence region capable of forming a minimum number of base pairs with other nucleotides of the nucleic acid strand. In some embodiments, there may be two or more nucleotides of the sequence region each capable of forming a minimum number of base pairs with other nucleotides of the nucleic acid strand. The minimum number of bases pairs a nucleotide can form with other nucleotides can be zero or one.

In some embodiments, if the first nucleotide forming the minimum number of base pairs with other nucleotides of the nucleic acid strand is not a guanine (G), the method introduces the first LNA modification at the first nucleotide. If the first nucleotide forming the minimum number of base pairs with other nucleotides of the nucleic acid strand is a G, the method continues to scan the sequence region until the next non-G base (e.g. A, C, or U) forming the minimum number of base pairs with other nucleotides of the nucleic acid strand is identified. The method then introduces the first LNA modification at the next non-G base. If all the nucleotides of the sequence region each forming the minimum number of base pairs with other nucleotides of the nucleic acid strand are G, the method can introduce the first LNA modification at the first G base of the sequence region that form the minimum number of base pairs with other nucleotides of the nucleic acid strand.

As described in greater details below, introducing a LNA modification comprises introducing a chemical bridge connecting two carbons of a nucleotide such as the 2′ and 4′ carbons. In some embodiments, the chemical bridge connecting the 2′ and 4′ carbons of a nucleotide can be a 2′-O, 4′-C methylene bridge or a 2′-O, 4′-C ethylene bridge. In some embodiments, the computing system can comprise one or more input elements (e.g. text boxes) allowing a user to define a specific LNA modification, such as the features of the chemical bridge to be introduced.

The method 100 proceeds from block 110 to block 112, where the computing system introduces a second LNA modification at a second nucleotide position of the sequence region. In some embodiments, the second nucleotide is placed at a non-G base (e.g. A, C, or U) of the sequence region.

In some embodiments, following the placement of the first LNA, the method continues along the sequence region to identify the second nucleotide where the second LNA modification can be placed. The second nucleotide can be at least two bases downstream from the first LNA modification introduced, such as two, three or four bases downstream from the first LNA modification introduced. In some embodiments, the second nucleotide can base pair with another nucleotide of the nucleic acid strand in the obtained internal secondary structure and/or self-duplex secondary structure. In some embodiments, the second nucleotide is unpaired with any nucleotide of the nucleic acid strand.

For example, in the method described herein, along the sequence region from the first nucleotide where the first LNA is placed, a next non-G nucleotide can be identified at least two bases downstream that is either unpaired or forms a single base pair with another nucleotide of the nucleic acid strand and introduce the second LNA at that base.

The method 100 proceeds from block 112 to block 114, where the computing system repeats the previous step in block 112 to identity one or more nucleotides suitable for placing LNAs until reaching the end position of the sequence region.

In some embodiments, following the placement of the second LNA, the method continues along the sequence region to identify the third nucleotide where the third LNA modification can be placed. The third nucleotide can be at least two bases downstream from the second LNA modification introduced, such as two, three or four bases downstream from the second LNA modification introduced. In some embodiments, the third nucleotide can base pair with another nucleotide of the nucleic acid strand in the obtained internal secondary structure and/or self-duplex secondary structure formed by the nucleic acid strand. In some embodiments, the third nucleotide is unpaired with any nucleotide of the nucleic acid strand. For example, the method can move along the sequence region from the second nucleotide where the second LNA is placed to identify a next non-G nucleotide at least two bases downstream that is either unpaired or forms a single base pair with another nucleotide of the nucleic acid strand and introduce the third LNA at that base. The method can repeat this process by placing one or more LNAs until reaching the end position of the sequence region.

The computing system then generates a plurality of sequence regions each comprising one or more LNA modifications. The modified sequence regions can be mapped to the input nucleic acid strand to provide a LNA-modified nucleic acid strand.

In some embodiments, about 10%-50% of the nucleotides of the nucleic acid strand are modified with LNA or analogues thereof. In some embodiments, any two LNA modifications of the plurality of LNA modifications are at least one nucleotide apart (e.g. one, two, or three nucleotides apart).

In some embodiments, to output the LNA-modified nucleic acid strand, the computing system can generate and/or cause to display a second UI comprising the information related to the generated LNA-modified nucleic acid strand. The second UI can also comprise a link (e.g. a web address) to the information related to the LNA-modified nucleic acid strand and/or an input element (e.g. a button) for receiving a user input or selection for exporting the information related to the LNA-modified nucleic acid strand.

The method 100 can end at block 116.

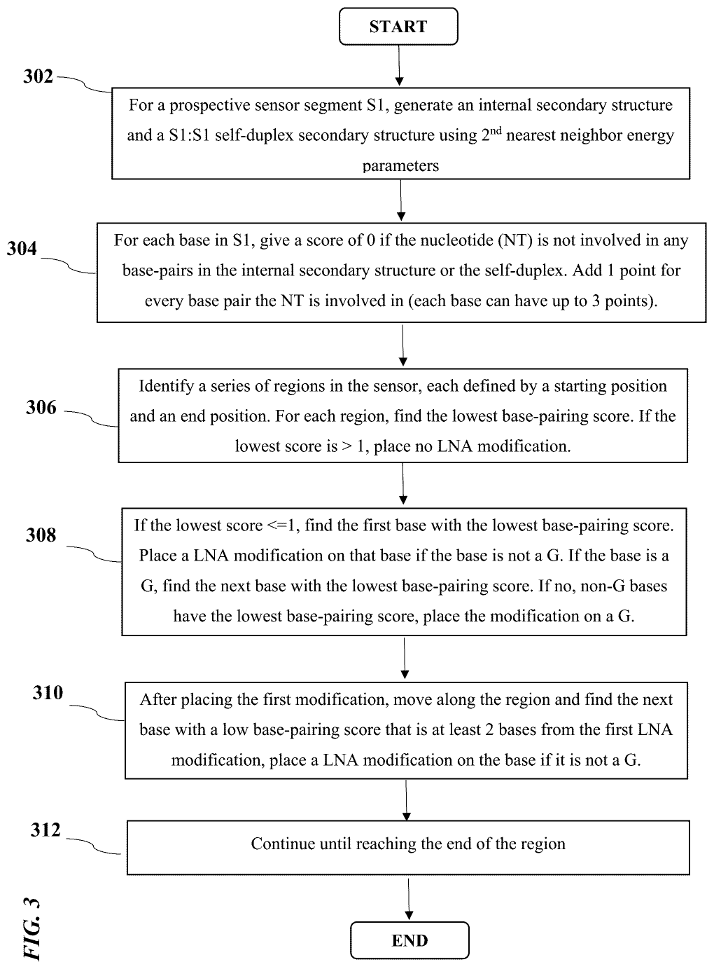

FIG. 3 is a schematic diagram showing a non-limiting exemplary workflow for placing LNAs in a conditionally activatable siRNA sensor strand. A computing system such as the computing system shown in FIG. 2 receives a sensor segment S1 and generates an internal secondary structure and a S1:S1 self-duplex structure using nearest neighbor energy parameters such as those in Nearest Neighbor Database (rna.urmc.rochester.edu/NNDB/index.html) (302). The computing system then assigns a base-pairing score for each base in S1 (304). For example, a nucleotide can be assigned a score of zero point if the nucleotide is not involved in any base-pairs in the internal secondary structure or the self-duplex secondary structure generated in block 302. A nucleotide can be assigned one point for every base pair the nucleotide is involved in. Each nucleotide can have up to three points, i.e. base pairing with up to 3 other nucleotides. The computing system then identifies a series of sequence regions from the sensor segment, each defined by a starting position and an end position (306). For each sequence region, the computing system identifies the lowest base-pairing score of the nucleotides. If the lowest base-pairing score of a sequence region is greater than 1, the sequence region is discarded, i.e. no LNA modification will be placed in this sequence region. If the lowest score of a sequence region is zero or one, the computing system identifies the first base with the lowest base-pairing score (e.g. zero) and places a LNA modification on that base if the base is not a G (308). If the base is a G, the computing system continues along the sequence region to identify the next base with the lowest base-paring score (e.g. zero) and places a LNA modification on that base. If no, non-G bases have the lowest base-pairing score (e.g. zero), the computing system places the modification on a base G, such as the first G of the sequence region having the lowest base-paring score (e.g. zero). After placing the first LNA, the computing system continues along the sequence region to identify the second base with a low base-pairing score (e.g. zero or one) that is at least two bases downstream from the first LNA modification and places the second LNA modification at the second base if the second base is not a G (310). After placing the second LNA, the computing system continues along the sequence region to identify one or more nucleotides with a low base-paring score (e.g. zero or one) that is at least two bases downstream from the previous LNA modification and places one or more LNA modifications at those bases until reaching the end position of the sequence region. The computing system then maps each of the LNA-modified regions to the sensor segment S1 to generate a LNA-modified sensor segment.

The generated LNA-modified sensor strand can be assembled with the other two component strands, a core strand and a passenger strand, in silico or in vitro to further test for secondary and tertiary structure assembly, conditional activation (e.g. by a trigger RNA), target specificity and off-target binding effects. Suitable software suites can be used to aid in the design and analysis of nucleic acid structures. For example, RNA secondary structure design software (e.g. Nupack, RNAstructure, RNAfold) can be used to check the formation of duplexes (e.g. the duplex formed by a core nucleic acid strand and a LNA-modified sensor nucleic acid strand) and to rank the thermodynamic stability of the duplexes. Computational simulation tools (e.g. molecular dynamics) can be employed to predict realistic molecular conformations of the nucleic acid complexes formed by the three components strands (e.g. the core strand, the passenger strand, and the LNA-modified sensor strand), and optionally in the presence of an input nucleic acid strand.

The LNA-modified strands (e.g. the core strand, the passenger strand, and/or the sensor strand) can also be synthesized and the synthesized oligonucleotides can be allowed to form a nucleic acid tertiary structure under a desirable physiological condition (e.g. 1× phosphate buffered saline at pH 7.4 with 1 mM concentration of MgCl2 at 37° C.). The formed tertiary structure can be analyzed using standard methods known in the art such as chemical mapping or NMR.

In some embodiments, the LNA-modified sensor strand can be tested in cell culture using an appropriate cell line representative of the targeted tissue. For example, a LNA-modified sensor strand can be combined with a core nucleic acid strand and a passenger nucleic acid strand under a suitable experimental condition to allow the assembly of a nucleic acid complex by thermally annealing the three strands. The core nucleic acid strand and/or the passenger nucleic acid strand can be either LNA-modified or unmodified. The assembled nucleic acid complexes can be transfected into an appropriate cell line (e.g. HCT 116 cells) containing a reporter vector (e.g. a dual luciferase vector) carrying a target RNA. The specific conditions and experimental protocols used are described in the examples (see e.g., Examples 1-3).

In some embodiments, according to the test results, the LNA-modified sensor strand can be further modified in the same or different computing system by introducing or removing one or more chemical modifications, mismatches, wobble parings, as necessary, until a desired structure is obtained. The chemical modification can comprise any phosphonate modification, ribose modification, base modification, and/or terminal moieties as described in greater details in the sections below. For example, the LNA-modified sensor strand can be further modified by attaching a terminal moiety at the 5′ terminus, the 3′ terminus, or both. The terminal moiety can comprise a ligand, a fluorophore, a exonuclease, a fatty acid, a Cy3, an inverted dT attached to a tri-ethylene glycol, or a combination thereof.

The method described herein can further comprise producing the generated LNA-modified strand. The LNA-modified strand generated using the method described herein can be produced using, for example, chemical synthesis. The LNA-modified strand can be synthesized using standard methods for oligonucleotide synthesis well-known in the art including, for example, Oligonucleotide Synthesis by Herdewijin, Piet (2005) and Modified oligonucleotides: Synthesis and Strategy for Users, by Verma and Eckstein, Annul Rev. Biochem. (1998): 67:99-134, the contents of which are incorporated herein by reference in their entirety.

### Nucleic Acid Complexes

Provided herein is a nucleic acid complex comprising a passenger nucleic acid strand, a core nucleic acid strand, and a sensor nucleic acid strand such as a LNA-modified sensor strand generated using the method disclosed herein.

The nucleic acid complex can be conditionally activated upon a complementary binding to an input nucleic acid strand (e.g. a mRNA of a disease biomarker gene specific to a target cell (e.g., disease-related cells)) through a sequence in a sensor nucleic acid strand of the nucleic acid complex. The activated nucleic acid complex can release the potent RNAi duplex formed by a core nucleic acid strand and a passenger nucleic acid strand, which can specifically inhibit or silence a target RNA. The target RNA can have a sequence independent from the input nucleic acid strand. The target RNA can be from a gene that is different from the gene that the input nucleic acid strand is from. In some embodiments, the target RNA is from a gene that is the same as the gene that the input nucleic acid strand is from.

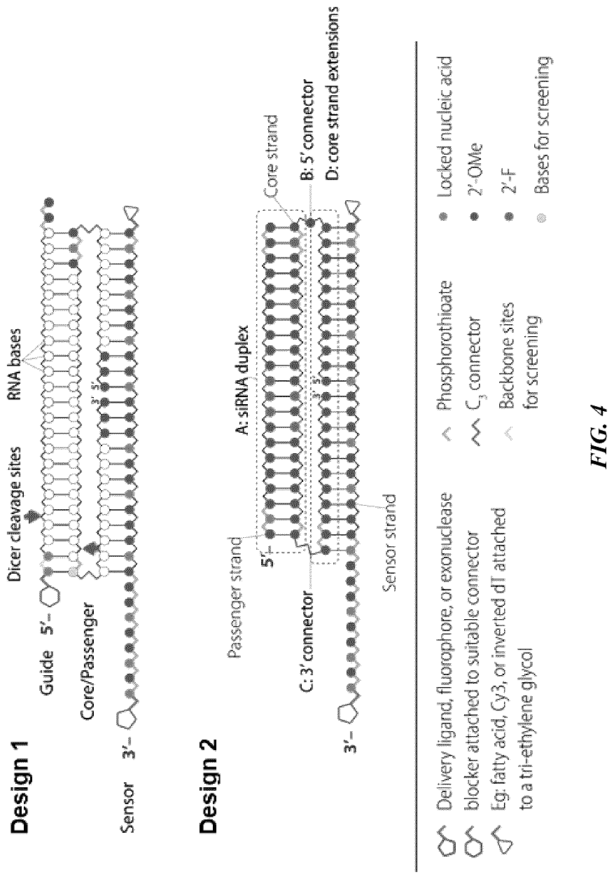

FIGS. 4-6 illustrate schematic representations of non-limiting exemplary nucleic acid complex constructs.

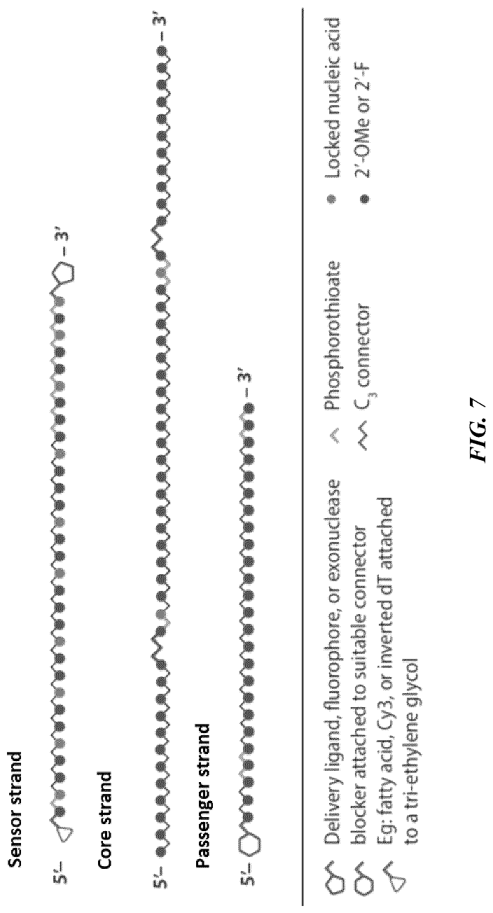

In some embodiments, the nucleic acid complexes described herein comprise a core nucleic acid strand, a passenger nucleic acid strand, and a sensor nucleic acid strand as shown in a non-limiting embodiment of FIG. 7. These three strands can base-pair with one another to form, for example, a RNAi duplex and a sensor duplex. One or more of the core nucleic acid strand, the passenger nucleic acid strand, and the sensor nucleic acid strand can be RNA analogs comprising modified nucleotides.

The term “nucleic acid duplex” as used herein refers to two single-stranded polynucleotides bound to each other through complementarily binding. The nucleic acid duplex can form a helical structure, such as a double-stranded RNA molecule, which is maintained largely by non-covalent bonding of base pairs between the two single-stranded polynucleotides and by base stacking interactions.

In some embodiments, the core nucleic acid strand of a nucleic acid complex herein described can comprise a 5′ region, a 3′ region, and a central region between the 5′ region and the 3′ region (see e.g., in FIG. 4). The central region of the core nucleic acid strand can be linked to the 5′ region and/or the 3′ region of the core nucleic acid strand via a connector. In some embodiments, the central region of the core nucleic acid strand is linked the 5′ region of the core nucleic acid strand via a 5′ connector. In some embodiments, the central region of the core nucleic acid strand is linked to the 3′ region of the core nucleic acid strand via a 3′ connector. The central region of the core nucleic acids strand is complementarily bound to the passenger nucleic acid strand to form a RNAi duplex. Not the entire sequence of the core nucleic acid strand is complementarily bound to the passenger nucleic acid strand. For example, the 5′ region and the 3′ region of the core nucleic acid strand is not complementarily bound to the passenger nucleic acid strand.

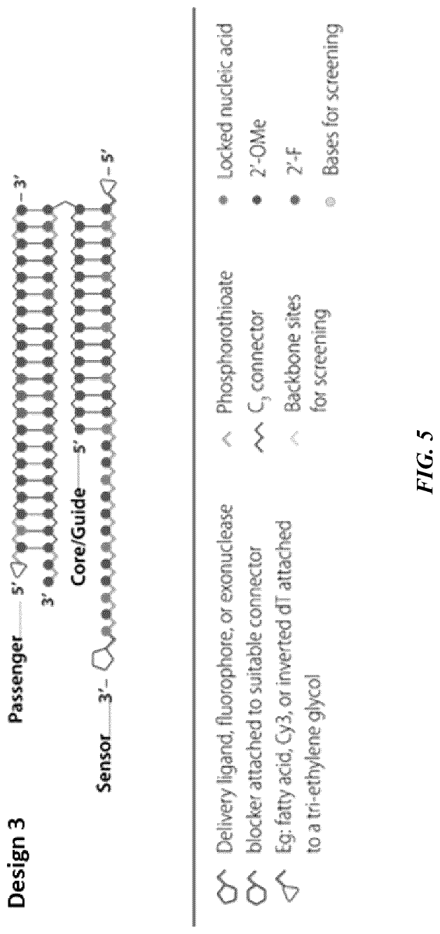

In some embodiments, the core nucleic acid strand can comprise two regions: a first region and a second region and the first region is at the 3′ direction of the second region (see e.g., FIG. 5). In other words, the first region is at the 3′ end of the core nucleic acid strand and the second region is at the 5′ end of the core nucleic acid strand. The first region of the core nucleic acid strand can be linked to the second region of the core nucleic acid strand via a connector, which can also be referred to as a 5′ connector. The 5′ connector can be a normal phosphodiester internucleoside linkage connecting two adjacent nucleotides. In some embodiments, the core nucleic acid strand only comprises one connector (e.g. 5′ connector) and does not comprise a 3′ connector. The first region of the core nucleic acid strand is complementarily bound to the passenger nucleic acid strand to form a RNAi duplex. Not the entire sequence of the core nucleic acid strand is complementarily bound to the passenger nucleic acid strand. For example, the second region of the core nucleic acid strand is not complementarily bound to the passenger nucleic acid strand. In some embodiments, the first region of the core nucleic acid strand is fully complementary to the passenger nucleic acid strand, thereby forming a RNAi duplex having a blunt end with no overhang at the 5′ and 3′ termini of the first region of the core nucleic acid strand. The core nucleic acid strand of the RNAi duplex can have a short overhang at the 3′ terminus (e.g. one, two, or three nucleosides), but the 3′ overhang does not extend back into the middle of the sensor duplex to bind with the sensor nucleic acid strand (see e.g., Design 3 in FIG. 5). In some embodiments, the core nucleic acid strand does not have any region at the 3′ of the first region of the core nucleic acid strand.

The core nucleic acid strand (e.g. the central region of Design 1 and Design 2 in FIG. 4 or the first region of Design 3 in FIG. 5) can comprise a sequence complementary to a target nucleic acid (e.g. a RNA to be silenced). The core nucleic acid strand of the nucleic acid complex therefore acts as a guide strand (antisense strand) and is used to base pair with a target RNA. The passenger nucleic acid strand can therefore comprise a sequence homologous to the same target nucleic acid.

Upon activation of the nucleic acid complex (e.g. binding to an input nucleic acid strand), the released RNAi duplex can complementarily bind a target nucleic acid through the binding between the target nucleic acid and the core nucleic acid strand. In some embodiments, the sequence complementary to a target RNA in the core nucleic acid strand can be about 10-35 nucleosides in length. In some embodiments, the core nucleic acid strand comprises 20-70 linked nucleosides, optionally 20-60 linked nucleosides.

In some embodiments, the sensor nucleic acid strand is complementarily bound to the 5′ region and the 3′ region of the core nucleic acid strand to form a sensor duplex (e.g. in FIG. 4). The sensor nucleic acid strand does not bind to the central region of the core nucleic acid strand. In some embodiments, the sensor nucleic acid strand is complementarily bound to the second region of the core nucleic acid strand to form a sensor duplex (e.g. in FIG. 5). The sensor nucleic acid strand does not bind to the first region of the core nucleic acid strand nor any region of the core nucleic acid strand that is 3′ of the first region of the core nucleic acid strand. The sensor nucleic acid strand also does not bind to the passenger nucleic acid strand.

The sensor nucleic acid strand can comprise a toehold or an overhang. The term “overhang” as used herein refers to a stretch of unpaired nucleotides that protrudes at one of the ends of a double-stranded polynucleotide (e.g. a duplex). An overhang can be on either strand of the polynucleotide and can be included at either the 3′ terminus of the strand (3′ overhang) or at the 5′ terminus of the strand (5′ overhang). The overhang can be at the 3′ terminus of the sensor nucleic acid strand. The overhang of the sensor nucleic acid strand does not bind to any region of the core nucleic acid strand. The overhang of the sensor nucleic acid strand can be about 5-20 nucleosides in length.

The sensor nucleic acid strand can comprise a sequence capable of binding to an input nucleic acid strand (e.g. a mRNA of a disease biomarker gene specific to a target cell, including a disease-related cell). Upon activation, the binding of the sensor nucleic acid strand to the input nucleic acid strand can cause displacement and subsequent release of the sensor nucleic acid strand from the core nucleic acid strand, thereby releasing the potent RNAi duplex and switching on the RNA interfering activity of the RNAi duplex. In the absence of an input nucleic acid strand or a detectable amount of the input nucleic acid strand, the nucleic acid complex herein described remains in an inactivated state (switched off) and the displacement of the sensor nucleic acid strand from the core nucleic acid strand does not take place. Therefore, the input nucleic acid strand can act as a trigger to activate (switch on) the RNA interfering activity of the nucleic acid complex (e.g. RNAi duplex).

The length of the RNAi duplex of the nucleic acid complex herein described can vary in different embodiments. In some embodiments, the length of the RNAi duplex can be 10-35 nucleotides, optionally 10-30 nucleotides. For example, the length of the RNAi duplex can be, 10, 11, 12, 13, 14, 15, 16, 17, 18, 19, 20, 21, 22, 23, 24, 25, 26, 27, 28, 29, 30, 31, 32, 33, 34, 35, or a range of any two of these values, nucleotides. In some embodiments, the length of the RNAi duplex can be 19-25 nucleotides, optionally 17-22 nucleotides.

The length of the sensor duplex of the nucleic acid complex herein described can vary in different embodiments. In some embodiments, the length of the sensor duplex can be 10-35 nucleotides, optionally 10-30 nucleotides. For example, the length of the sensor duplex can be, 10, 11, 12, 13, 14, 15, 16, 17, 18, 19, 20, 21, 22, 23, 24, 25, 26, 27, 28, 29, 30, 31, 32, 33, 34, 35, a range of any two of these values, nucleotides. In some embodiments, the length of the sensor duplex is about 14 nucleotides. In some embodiments, the sensor duplex has a relatively short length with respect to the RNAi duplex (see e.g., Design 3 in FIG. 5).

### Method of Producing a Nucleic Acid Complex

Provided herein also includes a method of producing a nucleic acid complex herein described. The component strands (e.g. the sensor strand, the passenger strand, and the core strand) of the nucleic acid complex described herein can be synthesized using standard methods for oligonucleotide synthesis well-known in the art. The component strands can also be purchased from commercial sources. The synthesized nucleic acid strands can be allowed to assembly into a nucleic acid complex and form its secondary structure under a desirable physiological condition as will be apparent to a skilled artisan.

In some embodiments, the method of producing a nucleic acid complex herein described comprises providing a core nucleic acid strand, a passenger nucleic acid strand, and a sensor nucleic acid strand, and contacting the core nucleic acid strand, the passenger nucleic acid strand, and the sensor nucleic acid strand under a condition for a period of time to form a nucleic acid complex. In some embodiments, one or more of the core nucleic acid strand, the passenger nucleic acid strand, and the sensor nucleic acid strand is a LNA-modified nucleic acid strand generated using the method herein described.

In some embodiments, the sensor nucleic acid strand is a LNA-modified strand. Accordingly, in some embodiments, the method of producing a nucleic acid complex herein described can comprise contacting a core nucleic acid strand, a passenger nucleic acid strand, and a LNA-modified sensor nucleic acid strand under a condition for a period of time to allow the assembly and formation of a nucleic acid complex.

In some embodiments, the nucleic acid complex is assembled by combining all three component strands under suitable experimental conditions such as 1× phosphate buffered saline (PBS) buffer and pH about 7.0. Assembly can take place by thermal annealing of the three strands at a suitable temperature using an annealing protocol identifiable to a skilled person (e.g. from 85° C. to 37° C. at about 1 degree Celsius per minute cooling rate). The term “thermal annealing” refers to a process of heating and cooling two or more single-stranded oligonucleotides with complementary sequences to allow for the formation of a nucleic acid assembly. For example, the component strands are heated to a temperature and held for a period of time (e.g. 85° C. for about 30 seconds) to disrupt any secondary structure within each strand, then followed by a slow cooling to facilitate hybridization as new hydrogen bonds form between the complementary sequences of the strands. The cooling rate can be about 0.02° C./second to about 0.2° C./second. For example, the strands can be cooled down from 85° C. to 50° C. at a cooling rate of 0.1° C./second, held for a period of time (e.g. 45 min) at 50° C., followed by a second cooling round from 50° C. to 37° C. at a cooling rate of 0.02° C./second. The strands can be further cooled down to a lower temperature (e.g. 4° C.) at a same or different cooling rate for temporary storage.

The nucleic acid complex can be assembled with or without purification. For assembly without purification, the sensor, core and passenger strands can be mixed at a suitable ratio (e.g. at a 1.1 to 1.0 to 1.1 molar ratio at 50 nM or 100 nM concentration in 1×PBS at pH˜7.0). The component strands can be combined at any suitable concentrations such as from 10 nM to 200 nM, optionally from 50 nM to 150 nM, optionally from 50 nM to 100 nM. In some embodiments, an excess of sensor and passenger strands are used to prevent production of constitutively active RNAi duplex formed by the core strand and the passenger strand. For assembly with purification, the sensor, core, and passenger strands can be combined and assembled at a nominal concentration (e.g. about 1 μM) using an annealing protocol identifiable to a person skilled in the art. Exemplary annealing and assembly protocols of the nucleic acid complex disclosed herein are described, for example, in WO 2020/033938 and U.S. Pat. No. 9,725,715B2, the contents of which are incorporated herein by reference.

The quality of the assembly is affected by the concentration and stoichiometric ratio of the strands used in the assembly, the duration of the annealing step, the temperature profile, the salt concentration, the pH, and other constituents of the assembly buffer, as will be understood by a person skilled in the art. The quality of the assembly can be assessed, for example, using non-denaturing gel electrophoresis (e.g. on 10% to 15% PAGE in 1×TBE at 4° C.). The assembled nucleic acid complex is typically presented as a single band with minimal detectable higher molecular weight aggregates or lower molecular weight fragments. The band corresponding to the assembled nucleic acid complexes can be cut from the gel. The assembled nucleic acid complexes can be extracted using a nucleic acid gel extraction kit or an electrodialysis extraction system identifiable by a skilled person.

Suitable software suites can be used to aid in the design and analysis of nucleic acid structures. For example, RNA secondary structure design software (e.g. Nupack, RNAstructure, RNAfold) can be used to check the formation of the duplexes and to rank the thermodynamic stability of the duplexes. Oligonucleotide design tools can be further used to optimize the placement of LNA modifications. The nucleic acid complex construct can be further modified, according to the test result, by introducing or removing chemical modifications or mismatches, as necessary, until the desired structure is obtained. Any of the regions of one or more of the strands in a nucleic acid complex herein described can be screened for an input nucleic acid sequence, a target nucleic acid sequence and/or chemical modifications herein described. For example, FIG. 8 illustrates a schematic representation of a non-limiting exemplary nucleic acid complex construct, highlighting in yellow the terminal bases that can be screened for chemical modifications such as LNA placements and other nucleotide analogs herein described.

The nucleic acid complexes produced using the methods herein described can be delivered to a target site, in vivo, ex vivo or in vitro, to modulate a target RNA. For example, a cell at the target site comprising a target RNA can be contacted with the nucleic acid complex herein described. Upon detection of an input nucleic acid strand, an input strand can bind to the overhang of the sensor nucleic acid strand to cause displacement of the sensor nucleic acid strand from the core nucleic acid strand to release the sequence complementary to the target RNA into the cell, thereby modulating the target RNA. The nucleic acid complexes generated can also be used to treat a disease or a condition in a subject or an individual. For example, the nucleic acid complex generated herein can be administered to the cells, tissues, and/or organs of a subject in need thereof in an effective amount via any suitable local or systemic administration route. Upon detection of an input nucleic acid strand, the input nucleic acid strand can bind to the overhang of the sensor nucleic acid strand to cause displacement of the sensor nucleic acid strand from the core nucleic acid strand to release the sequence complementary to a target RNA, thereby reducing the activity of the target RNA or protein expression from the target RNA in the subject to treat the disease or condition. Various delivery systems can be employed for delivering the nucleic acid complex herein described such as antibody conjugates, micelles, natural polysaccharides, peptides, synthetic cationic polymers, microparticles, lipid-based nanovectors among others as will be apparent to a skilled person.

### RNA Interference (RNAi)

The nucleic acid complexes produced using the method disclosed herein can be conditionally activated to switch from an assembled, inactivated state to an activated state to act on (e.g. degrade or inhibit) a specific target nucleic acid in response to the detection of an input nucleic acid (e.g. a nucleic acid sequence specific to a target cell, including a disease-related cell) having a sequence complementary to a sequence in the sensor nucleic acid strand of a nucleic acid complex.

In the assembled, inactivated configuration, the sensor nucleic acid strand of the nucleic acid complex inhibits enzymatic processing of the RNAi duplex, thereby keeping RNAi activity switched off.

In the event that an input nucleic acid strand complementary to the sensor nucleic acid strand of a nucleic acid complex is present, the input nucleic acid strand can activate the nucleic acid complex by inducing separation of the sensor nucleic acid strand from the core nucleic acid strand via toehold mediated strand displacement. Displacement can start from a toehold formed at the 3′ or 5′ terminus of the sensor nucleic acid strand (e.g. a 5′ toehold or a 3′ toehold) through a complementary binding between the input nucleic acid strand and a toehold of the sensor nucleic acid strand.

After removal of the sensor nucleic acid strand, the unpaired region(s) of the core nucleic acid strand become 3′ and/or 5′ overhangs that can be degraded by nucleases (e.g. exonuclease). This degradation stops at the 3′ end and 5′ end of the RNAi duplex due to the presence of chemically modified nucleotides and/or exonuclease cleavage-resistance moieties, thus rendering an active RNAi duplex for further endonuclease processing if needed and RNA-induced silencing complex (RISC) loading.

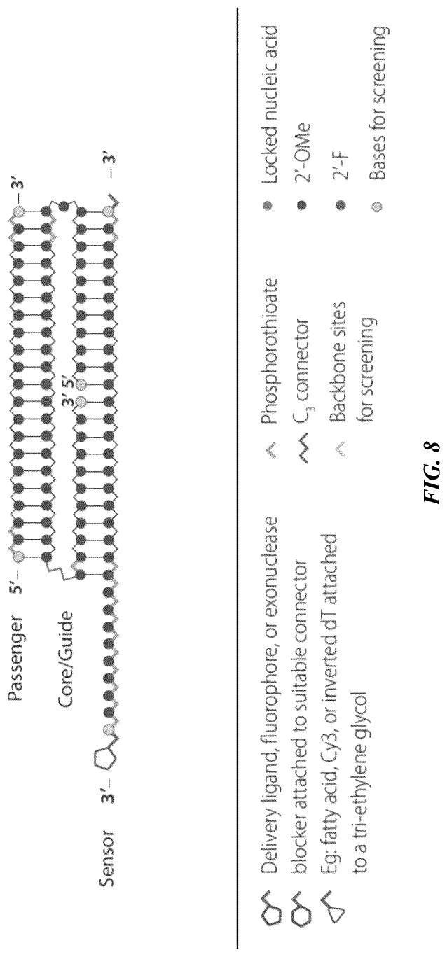

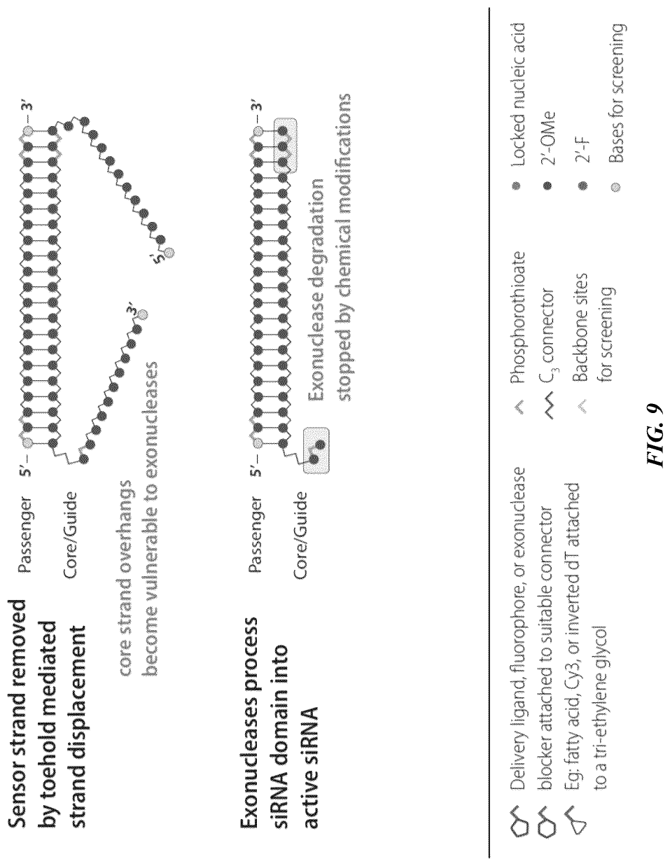

FIG. 9 is a schematic diagram showing the formation of an active RNAi duplex following the displacement of a sensor nucleic acid strand from a core nucleic acid strand and the degradation of the core nucleic acid strand overhangs.

RISC is a multiprotein complex that incorporates one strand of a siRNA or miRNA and uses the siRNA or miRNA as a template for recognizing complementary target nucleic acid. Once a target nucleic acid is identified, RISC activates RNase (e.g. Argonaute) and inhibits the target nucleic acid by cleavage. In some embodiments, Dicer is not required for loading the RNAi duplex into RISC.

The passenger nucleic acid strand is then discarded, while the core nucleic acid strand (e.g. the central region of the core nucleic acid strand) is incorporated into RICS. The core nucleic acid strand of the nucleic acid complex disclosed herein acts as a guide strand (antisense strand) and is used to base pair with a target RNA. The passenger nucleic acid strand acts as a protecting strand prior to the loading of the core nucleic acid strand into RICS. RICS uses the incorporated core nucleic acid strand as a template for recognizing a target RNA that has complementary sequence to the core nucleic acid strand, particularly the central region of the core nucleic acid strand. Upon binding to the target RNA, the catalytic component of RICS, Argonaute, is activated which can degrade the bound target RNA. The target RNA can be degraded or the translation of the target RNA can be inhibited.

In some embodiments, the nucleic acid complexes generated herein do not have a dicer cleavage site, and therefore the RNAi interference mediated by the nucleic acid complexes can bypass Dicer-mediated cleavage.

As will be apparent to a skilled artisan, Dicer is an endoribonuclease in the RNAse III family that can initiate the RNAi pathway by cleaving double-stranded RNA (dsRNA) molecule into short fragments of dsRNAs about 20-25 nucleotides in length.

In some embodiments, the nucleic acid complexes generated herein differentiate from the conditionally activated small interfering RNAs (Cond-siRNAs) disclosed in WO 2020/033938 in that the nucleic acid complexes generated herein can bypass the Dicer processing.

In some embodiments, the nucleic acid complexes generated herein have structural features that discourage the Dicer binding. In some embodiments, the RNAi duplex does not create a Dicer substrate. For example, the RNAi duplex formed by the passenger nucleic acid strand and the core nucleic acid strand do not have a 3′ and/or 5′ overhang, but instead forming a blunt end that can render the passenger nucleic acid strand unfavorable for Dicer binding. In some embodiments, the passenger nucleic acid strand has about 17-22 nucleotides in length, making it short enough to bypass Dicer cleavage. In some embodiments, the passenger nucleic acid strand does not have G/C rich bases to the 3′ and/or 5′ end of the passenger nucleic acid strand. In some embodiments, the passenger nucleic acid strand are attached to a terminal moiety to avoid Dicer binding.

Upon activation, the nucleic acid complex can inhibit a target nucleic acid in target cells, therefore resulting in a reduction or loss of expression of the target nucleic acid in the target cells. The target cells are cells associated or related to a disease or disorder. The term “associated to” “related to” as used herein refers to a relation between the cells and the disease or condition such that the occurrence of a disease or condition is accompanied by the occurrence of the target cells, which includes but is not limited to a cause-effect relation and sign/symptoms-disease relation. The target cells used herein typically have a detectable expression of an input nucleic acid.

In some embodiments, the expression of a target nucleic acid in target cells is inhibited about, at least, at least about, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or a number or a range between any of these values.

As used herein, inhibition of gene expression refers to the absence or observable decrease in the level of protein and/or mRNA product from a target gene in target cells. The degree of inhibition can be evaluated by examination of the expression level of the target gene as demonstrated in the examples.

In some embodiments, gene expression and/or the inhibition of target gene expression can be determined by use of a reporter or drug resistance gene whose protein product is easily assayed. Exemplary reporter genes include, but no limiting to, acetohydroxyacid synthase (AHAS), alkaline phosphatase (AP), beta galactosidase (LacZ), beta glucoronidase (GUS), chloramphenicol acetyltransferase (CAT), green fluorescent protein (GFP), horseradish peroxidase (HRP), luciferase (Luc), nopaline synthase (NOS), octopine synthase (OCS), and derivatives thereof. Multiple selectable markers are available that confer resistance to ampicillin, bleomycin, chloramphenicol, gentarnycin, hygromycin, kanamycin, lincomycin, methotrexate, phosphinothricin, puromycin, and tetracyclin. Quantitation of the amount of gene expression allows one to determine a degree of inhibition as compared to cells not treated with the nucleic acid complexes or treated with a negative or positive control. Various biochemical techniques may be employed as will be apparent to a skilled artisan such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS).

In some embodiments, the nucleic acid complexes produced herein exhibit improved switching performance and reduced off-target effects. The nucleic acid complexes generated herein can have a reduced unwanted RNAi activity when the nucleic acid complexes are in an inactivated state (switched off) and an enhanced RNAi activity when the nucleic acid complexes are activated upon detection of an input nucleic acid strand.

In some embodiments, the expression of a target nucleic acid in non-target cells (e.g. cells not having an input nucleic acid strand) is inhibited about, at most, or at most about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or a number or a range between any of these values. Non-target cells can comprise cells of the subject other than target cells.

In some embodiments, the nucleic acid complexes produced herein have an enhanced potency, thus capable of evoking an RNAi activity at low concentrations. Nonspecific, off-target effects and toxicity (e.g. undesired proinflammatory responses) can be minimized by using low concentrations of the nucleic acid complexes.

The concentration of the nucleic acid complexes produced herein can vary in different embodiments. In some embodiments, the nucleic acid complexes generated herein can be provided at a concentration of, about, at most, or at most about, 0.001 nM, 0.01 nM, 0.02 nM, 0.03 nM, 0.04 nM, 0.05 nM, 0.06 nM, 0.07 nM, 0.08 nM, 0.09 nM, 0.1 nM, 0.2 nM, 0.3 nM, 0.4 nM, 0.5 nM, 0.6 nM, 0.7 nM, 0.8 nM, 0.9 nM, 1.0 nM, 1.5 nM, 2.0 nM, 2.5 nM, 3.0 nM, 3.5 nM, 4.0 nM, 4.5 nM, 5.0 nM, 5.5 nM, 6.0 nM, 6.5 nM, 7.0 nM, 7.5 nM, 8.0 nM, 8.5 nM, 9.0 nM, 9.5 nM, 10 nM, 11 nM, 12 nM, 13 nM, 14 nM, 15 nM, 16 nM, 17 nM, 18 nM, 19 nM, 20 nM, 30 nM, 40 nM, 50 nM, or a number or a range between any two of these values. For example, the nucleic acid complexes generated herein can be provided at a concentration between about 0.1-10 nM, preferably between about 0.1-1.0 nM. In some embodiments, the nucleic acid complex generated herein has a transfection concentration at about 0.1 nM or lower.

The nucleic acid complex produced herein can allow lasting and consistently potent inhibition effects at low concentrations. For example, the nucleic acid complex can remain active for an extended period of time such as 1 hour, 2 hours, 6 hours, 12 hours, 24 hours, 36 hours, 48 hours, 60 hours, 72 hours, 84 hours, 96 hours, 5 days, 6 days, 7 days, two weeks, or a number or a range between any of these values, or more. In some embodiments, the nucleic acid complex can remain active for at least 12 hours, at least 24 hours, at least 36 hours, at least 48 hours, at least 60 hours, at least 72 hours, at least 84 hours, or at least 96 hours. In some embodiments, the nucleic acid complex can remain active for up to 30 days, up to 60 days, or up to 90 days.

### Chemical Modification

The nucleic acid strands (the core nucleic acid strand, the passenger nucleic acid strand, and/or the sensor nucleic acid strand) comprised in the nucleic acid complexes herein described can be further modified to introduce non-standard, modified nucleotides (nucleotide analog) or non-standard, modified nucleosides (nucleoside analog). The term “nucleotide analog” or “modified nucleotide” refers to a non-standard nucleotide comprising one or more modifications (e.g. chemical modifications), including non-naturally occurring ribonucleotides or deoxyribonucleotides. The term “nucleoside analog” or “modified nucleoside” refers to a non-standard nucleoside comprising one or more modification (e.g. chemical modification), including non-naturally occurring nucleosides other than cytidine, uridine, adenosine, guanosine, and thymidine. The modified nucleoside can be a modified nucleotide without a phosphate group. The chemical modifications can include replacement of one or more atoms or moieties with a different atom or a different moiety or functional group (e.g. methyl group or hydroxyl group).

The modifications are introduced to alter certain chemical properties of the nucleotide/nucleoside such as to increase or decrease thermodynamic stability, to increase resistance to nuclease degradation (e.g. exonuclease resistant), and/or to increase binding specificity and minimize off-target effects. For example, thermodynamic stability can be determined based on measurement of melting temperature Tₘ. A higher Tₘ can be associated with a more thermodynamically stable chemical entity.

In some embodiments, the modification can render one or more of the nucleic acid strands in the nucleic acid complex to resist exonuclease degradation/cleavage. The term “exonuclease” as used herein, indicates a type of enzyme that works by cleaving nucleotides one at a time from the end (exo) of a polynucleotide chain. A hydrolyzing reaction that breaks phosphodiester bonds at either the 3′ or the 5′ end occurs. A 3′ and 5′ exonuclease can degrade RNA and DNA in cells, and can degrade RNA and DNA in the interstitial space between cells and in plasma, with a high efficiency and a fast kinetic rate. A close relative is the endonuclease, which cleaves phosphodiester bonds in the middle (endo) of a polynucleotide chain. 3′ and 5′ exonuclease and exonucleolytic complexes can degrade RNA and DNA in cells, and can degrade RNA and DNA in the interstitial space between cells and in plasma. The term “exoribonuclease” as used herein, refers to exonuclease ribonucleases, which are enzymes that degrade RNA by removing terminal nucleotides from either the 5′ end or the 3′ end of the RNA molecule. Enzymes that remove nucleotides from the 5′ end are called 5′-3′ exoribonucleases, and enzymes that remove nucleotides from the 3′ end are called 3′-5′ exoribonucleases.

The modification can comprise phosphonate modification, ribose modification (in the sugar portion), and/or base modification. Preferred modified nucleotides used herein include sugar- and/or backbone-modified ribonucleotides.

In some embodiments, the modified nucleotide can comprise modifications to the sugar portion of the nucleotides. For example, the 2′ OH-group of a nucleotide can be replaced by a group selected from H, OR, R, F, Cl, Br, I, SH, SR, NH2, NIR, NR₂, COOR, or OR, wherein R is substituted or unsubstituted C₁-C₆ alkyl, alkenyl, alkynyl, aryl, etc. In some embodiments, the 2′ OH-group of a nucleotide or nucleoside is replaced by 2′ O-methyl group and the modified nucleotide or nucleoside is a 2′-O-methyl nucleotide or 2′-O-methyl nucleoside (2′-OMe). The 2′-O-methyl nucleotide or 2′-O-methyl nucleoside can be 2′-O-methyladenosine, 2′-O-methylguanosine, 2′-O-methyluridine, or 2′-O-methylcytidine. In some embodiments, the 2′ OH-group of a nucleotide is replaced by fluorine (F), and the modified nucleotide or nucleoside is a 2′-F nucleotide or 2′-F nucleoside (2′-deoxy-2′-fluoro or 2′-F). The 2′-F nucleotide or 2′-F nucleoside can be 2′-F-adenosine, 2′-F-guanosine, 2′-F-uridine, or 2′-F-cytidine. The modifications can also include other modifications such as nucleoside analog phosphoramidites. In some embodiments, glycol nucleic acids can be used.

In some embodiments, the modified nucleotide can comprise a modification in the phosphate group of the nucleotide, e.g. by substituting one or more of the oxygens of the phosphate group with sulfur or a methyl group. In some embodiments, one or more of the nonbridging oxygens of the phosphate group of a nucleotide is replaced by a sulfur.

In some embodiments, the nucleic acid strands herein described comprise one or more non-standard internucleoside linkage that is not a phosphodiester linkage. In some embodiments, the nucleic acid strands herein described comprise one or more phosphorothioate internucleoside linkages. The term “phosphorothioate linkage” (PS) as used herein, indicates a bond between nucleotides in which one of the nonbridging oxygens is replaced by a sulfur. In some embodiments, both nonbridging oxygens may be replaced by a sulfur (PS2). In some embodiments, one of the nonbridging oxygens may be replaced by a methyl group. The term “phosphodiester linkage” as described herein indicates the normal sugar phosphate backbone linkage in DNA and RNA wherein a phosphate bridges the two sugars. In some embodiments, the introduction of one or more phosphorothioate linkage in the core nucleic acid strand, the passenger nucleic acid strand, and/or the sensor nucleic acid strand can endow the modified nucleotides with increased resistance to nucleases (e.g. endonucleases and/or exonucleases).

In some embodiments, the modified nucleotide can comprise modifications to or substitution of the base portion of a nucleotide. For example, uridine and cytidine residues can be substituted with pseudouridine, 2-thiouridine, N6-methyladenosine, 5-methycytidine or other base analogs of uridine and cytidine residues. Adenosine can comprise modifications to Hoogsteen (e.g. 7-triazolo-8-aza-7-deazaadenosines) and/or Watson-Crick face of adenosine (e.g. N²-alkyl-2-aminopurines). Examples of adenosine analogs also include Hoogsteen or Watson-Crick face-localized N-ethylpiperidine triazole-modified adenosine analogs, N-ethylpiperidine 7-EAA triazole (e.g. 7-EAA, 7-ethynyl-8-aza-7-deazaadenosine) and other adenosine analogs identifiable to a person skilled in the art. Cytosine may be substituted with any suitable cytosine analogs identifiable to a person skilled in the art. For example, cytosine can be substituted with 6′-phenylpyrrolocytosine (PhpC) which has shown comparable base pairing fidelity, thermal stability and high fluorescence.

In some embodiments, one or more nucleotides in the nucleic acid complex disclosed herein can be substituted with a universal base. The term “universal base” refers to nucleotide analogs that form base pairs with each of the natural nucleotides with little discrimination between them. Examples of universal bases include, but are not limited to, C-phenyl, C-naphthyl and other aromatic derivatives, inosine, azole carboxamides, and nitroazole derivatives such as 3-nitropyrrole, 4-nitroindole, 5-nitroindole, and 6-nitroindole as known in the art (see e.g., Loakes, 2001, Nucleic Acids Research, 29, 2437-2447).

In some embodiments, base modification disclosed herein can reduce innate immune recognition while making the nucleic acid complex more resistant to nucleases. Examples of base modifications that can be used in the nucleic acid complex disclosed herein are also described, for example, in Hu et al. (Signal Transduction and targeted Therapy 5: 101 (2020)), the content of which is incorporated by reference in its entirety.

In some embodiments, the nucleic acid strands (the core nucleic acid strand, the passenger nucleic acid strand, and/or the sensor nucleic acid strand) comprised in the nucleic acid complexes herein described can comprise one or more locked nucleic acids or analogs thereof. For example, the sensor nucleic acid strand can be further modified to introduce locked nucleic acids using the method described herein. Exemplary locked nucleic acid analogs include, for example, their corresponding locked analog phosphoramidites and other derivatives apparent to a skilled artisan.

As used herein, the term “locked nucleic acids” (LNA) indicates a modified RNA nucleotide. The ribose moiety of an LNA nucleotide is modified with an extra bridge connecting the 2′ and 4′ carbons (a 2′-O, 4′-C methylene bridge). The bridge “locks” the ribose in the 3′-endo structural conformation and restricts the flexibility of the ribofuranose ring, thereby locking the structure into a rigid bicyclic formation. LNA nucleotides can be mixed with DNA or RNA bases in the oligonucleotide whenever desired. The incorporation of LNA into the nucleic acid complexes disclosed herein can increase the thermal stability (e.g. melting temperature), hybridization specificity of oligonucleotides as well as accuracies in allelic discrimination. LNA oligonucleotides display hybridization affinity toward complementary single-stranded RNA and complementary single- or double-stranded DNA. Additional information about LNA can be found, for example, at www.sigmaaldrich.com/technical-documents/articles/biology/locked-nucleic-acids-faq.html. In some embodiments, glycol nucleic acids can be used.

In some embodiments, the nucleic acid strands (the core nucleic acid strand, the passenger nucleic acid strand, and/or the sensor nucleic acid strand) comprised in the nucleic acid complexes herein described can comprise other chemically modified nucleotide or nucleoside with 2′-4′ bridging modifications. A 2′-4′ bridging modification refers to the introduction of a bridge connecting the 2′ and 4′ carbons of a nucleotide. The bridge can be a 2′-O, 4′-C methylene bridge (e.g. in LNA). The bridge can also be a 2′-O, 4′-C ethylene bridge (e.g. in ethylen-bridged nucleic acids (ENA)) or any other chemical linkage identifiable to a person skilled in the art.

In some embodiments, the introduction of LNA, analogues thereof, or other chemically modified nucleotides with 2′-4′ bridging modifications in the nucleic acid complex herein described can enhance hybridization stability as well as mismatch discrimination. For example, a nucleic acid complex comprising a sensor nucleic acid strand with LNA, analogues thereof, or other chemically modified nucleotides with 2′-4′ bridging modifications can have an enhanced sensitivity to distinguish between matched and mismatched input nucleic acid strand (e.g. in the complementary binding between an input nucleic acid strand and a sensor nucleic acid strand).

In some embodiments, one or more of the nucleic acid strands of the nucleic acid complex can comprise a chemical moiety linked to the 3′ and/or 5′ terminus of the strand. The terminal moiety can include one or more any suitable terminal linkers or modifications. For example, the terminal moiety can include a linker to link the oligonucleotide with another molecule or a particular surface (biotins, amino-modifiers, alkynes, thiol modifiers, azide, N-Hydroxysuccinimide, and cholesterol), a dye (e.g. fluorophore or a dark quencher), a fluorine modified ribose, a space (e.g. C3 spacer, Spacer 9, Spacer 18, dSpacer, tri-ethylene glycol spacer, hexa-ethylene glycol spacer), moieties and chemical modification involved in click chemistry (e.g. alkyne and azide moieties), and any linkers or terminal modifications that can be used to attach the 3′ and 5′ end to other chemical moieties such as antibodies, gold or other metallic nanoparticles, polymeric nanoparticles, dendrimer nanoparticles, small molecules, single chain or branched fatty acids, peptides, proteins, aptamers, and other nucleic acid strands and nucleic acid nanostructures. The terminal moiety can serve as a label capable of detection or a blocker to protect a single-stranded nucleic acid from nuclease degradation. Additional linkers and terminal modification that can be attached to the terminus of the sensor nucleic acid strand are described in www.idtdna.com/pages/products/custom-dna-rna/oligo-modifications and www.glenresearch.com/browse/labels-and-modifiers, the contents of which are incorporated herein by reference in their entirety.

Additional modifications to the nucleotides and/or nucleosides can also be introduced to one or more strands of the nucleic acid complex herein described, such as modifications described in Hu et al. (Signal Transduction and targeted Therapy 5: 101 (2020)), the content of which is incorporated by reference in its entirety.

### Ribose Modification

The percentage of the modified nucleosides of the nucleic acid complex can vary in different embodiments. In some embodiments, the percentage of the modified nucleosides of the nucleic acid complex herein described can be, be about, be at least, or be at least about 80%, 85%, 90%, or 95%. For example, percentage of the modified nucleosides of the nucleic acid complex herein described can be, be about, be at least, or be at least about 80%, 81%, 82%, 83%, 84%, 85%, 86%, 87%, 88%, 89%, 90%, 91%, 92%, 93%, 94%, 95%, 96%, 97%, 98%, 99%, or 100%, or a number or a range between any two of these values. In some embodiments, at least 90%, 91%, 92%, 93%, 94%, 95%, or a number or a range between any two of these values of the nucleotides of the nucleic acid complex are modified (e.g. are non-DNA and non-RNA). In some embodiments, all of the nucleotides of the nucleic acid complex are modified (e.g. are non-DNA and non-RNA).

The percentage of the modified nucleosides in one or more strands of the nucleic acid complex can vary in different embodiments. In some embodiments, the percentage of the modified nucleosides in a core nuclei acid strand herein described can be, be about, be at least, or be at least about 50%, 60%, 70%, 80%, 85%, 90%, or 95%. For example, the percentage of the modified nucleosides in a core nuclei acid strand herein described can be, be about, be at least, or be at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or a number or a range between any two of these values. In some embodiments, all of the nucleosides of a core nucleic acid strand are chemically modified.

In the embodiments where a core nucleic acid strand comprises a central region, a 3′ region and a 5′ region (see e.g., Design 2 in FIG. 4), the percentage of the modified nucleosides in the central region, the 3′ region, and/or the 5′ region of the core nuclei acid strand herein described can be, be about, be at least, or be at least about 80%, 85%, 90%, or 95%. For example, the percentage of the modified nucleosides in the central region, the 3′ region and/or the 5′ region of a core nuclei acid strand herein described can be, be about, be at least, or be at least about 80%, 81%, 82%, 83%, 84%, 85%, 86%, 87%, 88%, 89%, 90%, 91%, 92%, 93%, 94%, 95%, 96%, 97%, 98%, 99%, or 100%, or a number or a range between any two of these values. In some embodiments, all of the nucleosides of the 5′ region and/or the 3′ region of a core nucleic acid strand are chemically modified.

In the embodiments wherein a core nucleic acid strand comprises a first region and a second region (see e.g., Design 3 in FIG. 5), the percentage of the modified nucleosides in the first region and/or the second region of the core nuclei acid strand herein described can be, be about, be at least, or be at least about 50%, 60%, 70%, 80%, 85%, 90%, or 95%. For example, the percentage of the modified nucleosides in the first region and/or the second region of a core nuclei acid strand herein described can be, be about, be at least, or be at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or a number or a range between any two of these values. In some embodiments, all of the nucleosides of the first region and/or the second region of a core nucleic acid strand are chemically modified.

The percentage of the modified nucleosides in a passenger nuclei acid strand herein described can be, be about, be at least, or be at least about 8050%, 60%, 70%, 80%, 85%, 90%, or 95%. For example, the percentage of the modified nucleosides in a passenger nuclei acid strand herein described can be, be about, be at least, or be at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or a number or a range between any two of these values. In some embodiments, all of the nucleosides of a passenger nucleic acid strand are chemically modified.

In some embodiments, the percentage of the modified nucleosides in a sensor nuclei acid strand herein described can be, be about, be at least, or be at least about 50%, 60%, 70%, 80%, 85%, 90%, or 95%. In some embodiments, the percentage of the modified nucleosides in a sensor nuclei acid strand herein described can be, be about, be at least, or be at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or a number or a range between any two of these values. In some embodiments, all of the nucleosides of a sensor nucleic acid strand are chemically modified.

The modified nucleosides in one or more of the core nucleic acid strand, the passenger nucleic acid strand, and the sensor nucleic acid strand can comprise 2′-O-methyl nucleoside and/or 2′-F nucleoside.

In some embodiments, the percentage of 2′-O-methyl nucleoside and/or 2′-F nucleoside in the nucleic acid complex herein described can be, be about, be at least, be at least about, be at most, or be at most about 10%-50%. For example, the percentage of 2′-O-methyl nucleoside and/or 2′-F nucleoside in the nucleic acid complex herein described can be, be about, be at least, be at least about, be at most, or be at most about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or a number or a range between any two of these values.

In some embodiments, the percentage of 2′-O-methyl nucleoside and/or 2′-F nucleoside in a core nucleic acid strand herein described can be, be about, be at least, be at least about, be at most, or be at most about 10%-50%. For example, the percentage of 2′-O-methyl nucleoside and/or 2′-F nucleoside in a core nucleic acid strand herein described can be, be about, be at least, be at least about, be at most, or be at most about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or a number or a range between any two of these values.

In some embodiments, the percentage of 2′-O-methyl nucleoside and/or 2′-F nucleoside in a passenger nucleic acid strand herein described can be, be about, be at least, be at least about, be at most, or be at most about 10%-50%. For example, the percentage of 2′-O-methyl nucleoside and/or 2′-F nucleoside in a passenger nucleic acid strand herein described can be, be about, be at least, be at least about, be at most, or be at most about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or a number or a range between any two of these values.

In some embodiments, the percentage of 2′-O-methyl nucleoside and/or 2′-F nucleoside in a sensor nucleic acid strand herein described can be, be about, be at least, be at least about, be at most, or be at most about 10%-50%. For example, the percentage of 2′-O-methyl nucleoside and/or 2′-F nucleoside in a sensor nucleic acid strand herein described can be, be about, be at least, be at least about, be at most, or be at most about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or a number or a range between any two of these values.

### Phosphate Modification

The percentage of phosphate modification to the nucleotides in the nucleic acid complex described herein can vary in different embodiments. In some embodiments, the phosphate modification comprises or is a phosphorothioate internucleoside linkage. In some embodiments, the percentage of phosphorothioate internucleoside linkages in a core nucleic acid strand is less than 5%, less than 10%, less than 25%, less than 50%, or a number or a range between any two of these values. For example, percentage of phosphorothioate internucleoside linkages in a core nucleic acid strand is about, less than, or less than about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50% or a number or a range between any two of these values. In some embodiments, the core nucleic acid strand comprises no more than two phosphorothioate internucleoside linkages. In some embodiments, the core nucleic acid strand does not comprise a phosphorothioate internucleoside linkage modification.

The percentage of phosphodiester internucleoside linkages in a core nucleic acid strand can be, be about, be at least, or be at least about, 50%, 80% or 95%, or a number or a range between any two of these values. For example, percentage of phosphodiester internucleoside linkages in a core nucleic acid strand is about, at least, or at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or a number or a range between any two of these values. In some embodiments, all the internucleoside linkages in the core nucleic acid strand are phosphodiester internucleoside linkage.

In some embodiments wherein a core nucleic acid strand comprises a central region, a 3′ region, and a 5′ region (see e.g., Design 2 in FIG. 4), the 5′ terminus of the central region of the core nucleic acid strand comprises at least one phosphorothioate internucleoside linkage (e.g. one, two or three phosphorothioate internucleoside linkage). In some embodiments, the 3′ terminus of the central region of the core nucleic acid strand comprises at least one phosphorothioate internucleoside linkage (e.g. one, two or three phosphorothioate internucleoside linkage). In some embodiments, each of the 5′ terminus of the central region of the core nucleic acid strand and the 3′ terminus of the central region of the core nucleic acid strand independently comprises one or more phosphorothioate internucleoside linkages (e.g. one, two or three phosphorothioate internucleoside linkage). In some embodiments, the central region of the core nucleic acid strand does not comprise phosphorothioate internucleoside linkages except for the phosphorothioate internucleoside linkage(s) between two or three nucleosides at the 5′ terminus, 3′ terminus, or both, of the central region. In some embodiments, the internucleoside linkages between the one to three nucleotides (e.g. one, two, or three nucleotides) adjacent to the 3′ of the 5′ connector of the core nucleic acid strand are phosphorothioate internucleoside linkages. In some embodiments, the internucleoside linkages between the one or two nucleotides adjacent to the 5′ of the 3′ connector of the core nucleic acid strand are phosphorothioate internucleoside linkages. In some embodiments, the internucleoside linkages between the one to three nucleotides (e.g. one, two, or three nucleotides) adjacent to the 3′ of the 3′ connector of the core nucleic acid strand are phosphorothioate internucleoside linkages. In some embodiments, the 3′ region of the core nucleic acid strand does not comprise phosphorothioate internucleoside linkages except for the phosphorothioate internucleoside linkage(s) between the one to three nucleotides (e.g. one, two, or three nucleotides) adjacent to the 3′ of the 3′ connector of the core nucleic acid strand. In some embodiments, the 5′ region of the core nucleic acid strand does not comprise phosphorothioate internucleoside linkages.

In some embodiments wherein a core nucleic acid strand comprises a first region and a second region (see e.g., Design 3 in FIG. 5), the 3′ terminus of the first region of the core nucleic acid strand comprises at least one phosphorothioate internucleoside linkage (e.g. one, two or three phosphorothioate internucleoside linkage). The phosphorothioate internucleoside linkage can be between the last two, three, or four nucleosides at the 3′ terminus of the first region of the core nucleic acid strand. In some embodiments, the 5′ terminus of the first region of the core nucleic acid strand comprises at least one phosphorothioate internucleoside linkage (e.g. one, two or three phosphorothioate internucleoside linkage). The phosphorothioate internucleoside linkage can be between the last two, three, or four nucleosides at the 5′ terminus of the first region of the core nucleic acid strand. In some embodiments, each of the 5′ terminus of the first region of the core nucleic acid strand and the 3′ terminus of the first region of the core nucleic acid strand independently comprises one or more phosphorothioate internucleoside linkages (e.g. one, two or three phosphorothioate internucleoside linkage). In some embodiments, the first region of the core nucleic acid strand does not comprise phosphorothioate internucleoside linkages except for the phosphorothioate internucleoside linkage(s) between the last two or three nucleosides at the 5′ terminus, 3′ terminus, or both, of the first region. For example, the first region of the core nucleic acid strand does not comprise phosphorothioate internucleoside linkages except for the phosphorothioate internucleoside linkage(s) between the last three nucleosides at the 5′ terminus and the last three nucleosides 3′ terminus of the first region. In some embodiments, the percentage of phosphorothioate internucleoside linkages in the second region of a core nucleic acid strand is less than 5%, less than 10%, or a number or a range between any two of these values. In some embodiments, the second region of a core nucleic acid strand does not comprise phosphorothioate internucleoside linkages.

In some embodiments, the passenger nucleic acid strand comprises one or more phosphorothioate internucleoside linkage. The percentage of phosphorothioate internucleoside linkages in a passenger nucleic acid strand is less than 5%, less than 10%, less than 25%, less than 50%, or a number or a range between any two of these values. For example, percentage of phosphorothioate internucleoside linkages in a passenger nucleic acid strand is about, less than, or less than about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50% or a number or a range between any two of these values.

In some embodiments, the 5′ terminus of the passenger nucleic acid strand comprises at least one phosphorothioate internucleoside linkage (e.g. one, two, or three phosphorothioate internucleoside linkage). In some embodiments, the 3′ terminus of the passenger nucleic acid strand comprises at least one phosphorothioate internucleoside linkage (e.g. one, two, or three phosphorothioate internucleoside linkage). In some embodiments, the passenger nucleic acid strand does not comprise phosphorothioate internucleoside linkages except for the phosphorothioate internucleoside linkage(s) between the last two, three, or four nucleosides at the 5′ terminus, 3′ terminus, or both, of the passenger nucleic acid strand. In some embodiments, the passenger nucleic acid strand does not comprise phosphorothioate internucleoside linkages except for the phosphorothioate internucleoside linkage(s) between the last two to three nucleosides at the 5′ terminus and the last two to three nucleosides at 3′ terminus of the passenger nucleic acid strand.

In some embodiments, the sensor nucleic acid strand comprises one or more phosphorothioate internucleoside linkage. The percentage of phosphorothioate internucleoside linkages in a sensor nucleic acid strand can be less than 5%, less than 10%, less than 25%, less than 50%, or a number or a range between any two of these values. For example, percentage of phosphorothioate internucleoside linkages in a sensor nucleic acid strand is about, less than, or less than about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50% or a number or a range between any two of these values.

In some embodiments, the 5′ terminus of the sensor nucleic acid strand comprises at least one phosphorothioate internucleoside linkage (e.g. one, two or three phosphorothioate internucleoside linkage). In some embodiments, the 3′ terminus of the sensor nucleic acid strand comprises at least one phosphorothioate internucleoside linkage (e.g. one to twenty phosphorothioate internucleoside linkage. In some embodiments, each of the 5′ terminus of the sensor nucleic acid strand and the 3′ terminus of the sensor nucleic acid strand independently comprises one or more phosphorothioate internucleoside linkages (e.g. one, two or three at the 5′ terminus or one to twenty at the 3′ terminus). In some embodiments, the sensor nucleic acid strand does not comprise phosphorothioate internucleoside linkages except for the phosphorothioate internucleoside linkage(s) at the 5′ terminus, 3′ terminus, or both, of the sensor nucleic acid strand. In some embodiments, the phosphorothioate internucleoside linkages at the 3′ terminus of the sensor nucleic acid strand are in the singled-stranded overhang of the sensor nucleic acid strand.

### LNA, Analogues Thereof and 2′-4′Bridging Modification

The percentage of the LNA or analogues thereof of the nucleic acid complex can vary in different embodiments. In some embodiments, the percentage of the LNA or analogues thereof of the nucleic acid complex herein described can be about 10%-50%. For example, the percentage of the LNA or analogues thereof of the nucleic acid complex herein described can be about, at most, at most about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50% or a number or a range between any two of these values.

The percentage of the LNA or analogues thereof in one or more strands of the nucleic acid complex can vary in different embodiments. In some embodiments, the percentage of the LNA or analogues thereof in a core nucleic acid strand herein described can be, be about, be at most, or be at most about 5%, 10%, or 15%. For example, the percentage of the LNA or analogues thereof of a core nucleic acid strand herein described can be, be about, be at most, or be at most about 5%, 6%, 7%, 8%, 9%, 10%, 11%, 12%, 13%, 14%, 15%, or a number or a range between any two of these values.

In some embodiments, the percentage of the LNA or analogues thereof in a passenger nucleic acid strand herein described can be, be about, be at most, or be at most about 5%, 10%, or 15%. For example, the percentage of the LNA or analogues thereof of a passenger nucleic acid strand herein described can be, be about, be at most, or be at most about 5%, 6%, 7%, 8%, 9%, 10%, 11%, 12%, 13%, 14%, 15%, or a number or a range between any two of these values. In some embodiments, a percentage of the LNA or analogues thereof in a passenger nucleic acid strand herein described greater than 5%, greater than 10%, or greater than 15% can decrease the RNAi activity of the nucleic acid complex (see e.g., Example 1).

In some embodiments, the percentage of the LNA or analogues thereof in a sensor nucleic acid strand herein described can be, be about, be at least, be at least about, be at most, or be at most about 10%-50%. For example, the percentage of the LNA or analogues thereof of a sensor nucleic acid strand herein described can be, be about, be at least, be at least about, be at most, or be at most about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or a number or a range between any two of these values.

The percentage of 2′-4′ bridging modification of the nucleic acid complex can vary in different embodiments. In some embodiments, the percentage of the 2′-4′ bridging modification of the nucleic acid complex herein described can be about 10%-50%. For example, the percentage of the 2′-4′ bridging modification of the nucleic acid complex herein described can be about, at most, at most about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50% or a number or a range between any two of these values.

### Core Strand

A core nucleic acid strand of a nucleic acid complex herein described can comprise a region complementarily bound to a passenger nucleic acid strand to form a RNAi duplex and one or more regions complementarily bound to a sensor nucleic acid strand to form a sensor duplex.

In some embodiments, the core nucleic acid strand can comprise a 5′ region, a 3′ region, and a central region between the 5′ region and the 3′ region (see e.g., FIG. 4). The central region is complementarily bound to a passenger nucleic acid strand and the 3′ and 5′ regions are complementarily bound to a sensor nucleic acid strand. Each of the 5′ region, the 3′ region, and the central region can be directly adjacent to each other, that is no nucleotide between the two adjacent regions. In some embodiments, the 3′ end of the 5′ region can be 1, 2, 3, 4, 5, 8, 9, 10, 11, 12, 13, 14, 15, 20, or a number or a range between any two of these values, nucleotides away from the 5′ end of the central region. In some embodiments, the 5′ end of the 3′ region can be 1, 2, 3, 4, 5, 8, 9, 10, 11, 12, 13, 14, 15, 20, or a number or a range between any two of these values, nucleotides away from 3′ of the central region. The length of the central region of the core nucleic acid strand can vary in different embodiments. In some embodiments, the central region of the core nucleic acid strand comprises 10-35 linked nucleosides. For example, the central region of the core nucleic acid strand can comprise 10, 11, 12, 13, 14, 15, 16, 17, 18, 19, 20, 21, 22, 23, 24, 25, 26, 27, 28, 29, 30, 31, 32, 33, 34, or 35 linked nucleosides. The 3′ region and the 5′ region of the core nucleic acid strand can have a same length or a different length. The length of the 3′ region and the 5′ region of the core nucleic acid strand can vary in different embodiments. In some embodiments, the length of the 3′ region and the 5′ region of the core nucleic acid strand comprises 2-33 linked nucleosides. For example, the 3′ region and the 5′ region of the core nucleic acid strand can comprise 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33 linked nucleosides.

In some embodiments, a core nucleic acid strand can comprise a first region and a second region (e.g. Design 3 in FIG. 5). The first region is at the 3′ direction of the second region. The first region is complementarily bound to a passenger nucleic acid strand and the second region is complementarily bound to a sensor nucleic acid strand. The length of the first region of the core nucleic acid strand can vary in different embodiments. In some embodiments, the first region of the core nucleic acid strand comprises 10-30 linked nucleosides. For example, the first region of the core nucleic acid strand can comprise 10, 11, 12, 13, 14, 15, 16, 17, 18, 19, 20, 21, 22, 23, 24, 25, 26, 27, 28, 29, or 30, linked nucleosides. In some embodiments, the first region of the core nucleic acid strand comprises 17-22 linked nucleosides. The length of the second region of the core nucleic acid strand can vary in different embodiments. In some embodiments, the length of the second region of the core nucleic acid strand comprises 10-30 linked nucleosides. For example, the second region of the core nucleic acid strand can comprise 10, 11, 12, 13, 14, 15, 16, 17, 18, 19, 20, 21, 22, 23, 24, 25, 26, 27, 28, 29, or 30, linked nucleosides. The first region and the second region of the core nucleic acid strand can have a same length or a different length. In some embodiments, the second region of the core nucleic acid strand has a relatively short length with respect to the first region of the core nucleic acid strand. In some embodiments, the second region of the core nucleic acid strand has about 14 linked nucleosides.

The length of the core nucleic acid strand can vary in different embodiments. In some embodiments, the core nucleic acid strand comprises 20-70 linked nucleosides, optionally 20-60 linked nucleosides. For example, the core nucleic acid strand can comprise 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 linked nucleosides.

The region of the core nucleic acid strand complementarily bound to the passenger nucleic acid strand (e.g. the central region or the first region) comprises a sequence complementary to a target RNA. The length of the sequence complementary to a target RNA can vary in different embodiments. In some embodiments, the sequence complementary to a target RNA is 10-35 nucleotides in length. For example, the sequence complementary to a target RNA is 10, 11, 12, 13, 14, 15, 16, 17, 18, 19, 20, 21, 22, 23, 24, 25, 26, 27, 28, 29, 30, 31, 32, 33, 34, or 35, nucleotides in length. In some embodiments, the sequence complementary to a target RNA is 10-21 nucleotides in length.

The core nucleic acid strand (e.g. the central region or the first region) comprises a sequence complementary to a passenger nucleic acid strand. The length of the sequence complementary to a passenger nucleic acid strand can vary in different embodiments. In some embodiments, the sequence complementary to a passenger nucleic acid strand is 19-25 nucleotides in length, optionally 17-22 nucleotides in length. For example, the sequence complementary to a passenger nucleic acid strand is 19, 20, 21, 22, 23, 24, or 25 nucleotides in length. In some embodiments, the sequence of the core nucleic acid strand complementary to a passenger nucleic acid strand is about 21 nucleotides in length.

In some embodiments, each of the regions in the core nucleic acid strand is linked to its adjacent region via a connector. For example, the central region of the core nucleic acid strand is linked the 5′ region of the core nucleic acid strand via a 5′ connector. In some embodiments, the central region of the core nucleic acid strand is linked to the 3′ region of the core nucleic acid strand via a 3′ connector. In some embodiments, the first region of the core nucleic acid strand is linked the second region of the core nucleic acid strand via a 5′ connector. In some embodiments, the core nucleic acid strand only comprises one connector (e.g. 5′ connector) and does not comprise a 3′ connector.

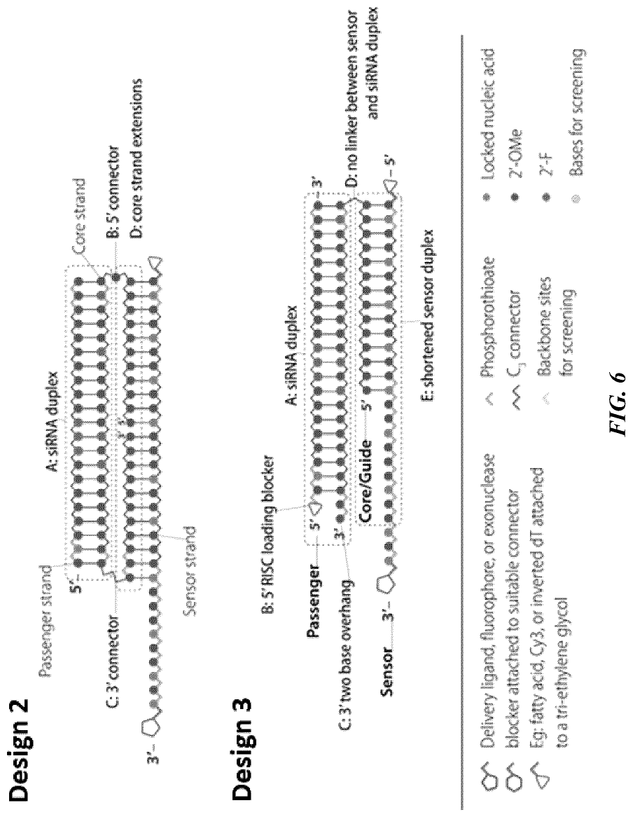

The 5′ connector and/or 3′ connector can comprise a three-carbon linker (C₃ linker), a nucleotide, any modified nucleotide described herein, or any moiety that can resist exonuclease cleavage when the core nucleic acid strand is single-stranded (e.g. after displacement of the sensor nucleic acid strand from the core nucleic acid strand). For example, the 5′ connector and/or the 3′ connector can comprise a 2′-F nucleotide such as 2′-F-adenosine, 2′-F-guanosine, 2′-F-uridine, or 2′-F-cytidine. The 5′ connector and/or the 3′ connector can comprise a 2′-O-methyl nucleotide such as 2′-O-methyladenosine, 2′-O-methylguanosine, 2′-O-methyluridine, or 2′-O-methylcytidine. The 5′ connector and/or the 3′ connector can comprise a naturally occurring nucleotide such as cytidine, uridine, adenosine, or guanosine. The 5′ connector and/or the 3′ connector of the core nucleic acid strand can comprise a phosphodiester linkage (phosphodiester 5′ and 3′ connection) cleavable by an exonuclease when in a single-stranded form. The 5′ connector and/or the 3′ connector of the core nucleic acid strand can comprise any suitable moiety that can resist exonuclease cleavage when in single-stranded form. In some embodiments, the 5′ connector of the core nucleic acid strand comprises no linker molecule except for the normal phosphodiester linkage connecting two adjacent nucleosides (see e.g., Design 3 shown in FIGS. 5-6).

The 5′ connector can comprise or is, a C₃ 3-carbon linker, a nucleotide, a modified nucleotide (2′-O-methyl nucleotide, 2′-F nucleotide), a nucleotide with a phosphodiester 5′ and 3′ connection cleavable by an exonuclease when in a single stranded form, or a combination thereof. In some embodiments, the 5′ connector can comprise or is a 2′-O-methyl nucleotide such as 2′-O-methyladenosine, 2′-O-methylguanosine, 2′-O-methyluridine, or 2′-O-methylcytidine. In some embodiments, the 5′ connector can comprise or is 2′-F nucleotide such as 2′-F-adenosine, 2′-F-guanosine, 2′-F-uridine, or 2′-F-cytidine.

The 3′ connector comprises or is, a C₃ 3-carbon linker, a nucleotide, a modified nucleotide, an exonuclease cleavage-resistant moiety when in a single stranded form, or a combination thereof. In some embodiments, the 3′ connector can comprise or is a 2′-O-methyl nucleotide such as 2′-O-methyladenosine, 2′-O-methylguanosine, 2′-O-methyluridine, or 2′-O-methylcytidine.

In some embodiments, the 3′ connector comprises or is a 2′-O-methyl nucleotide such as 2′-O-methyladenosine, 2′-O-methylguanosine, 2′-O-methyluridine, or 2′-O-methylcytidine and the 5′ connector comprises or is a 2′-O-methyl nucleotide such as 2′-O-methyladenosine, 2′-O-methylguanosine, 2′-O-methyluridine, or 2′-O-methylcytidine

In some embodiments, the 5′ connector of the core nucleic acid strand does not comprise or is not a C₃ 3-carbon linker. In some embodiments, the 3′ connector of the core nucleic acid strand comprises or is a C₃ 3-carbon linker. In some embodiments, it is advantageous to not have a C₃ 3-carbon linker as the 5′ connector. In some embodiments, it is advantageous to have a C₃ 3-carbon linker as the 3′ connector. In some embodiments, the 5′ connector of the core nucleic acid strand does not comprise or is not a C₃ 3-carbon linker, while the 3′ connector of the core nucleic acid strand comprises or is a C₃ 3-carbon linker.

In some embodiments, a nucleic acid complex not having a C₃ 3-carbon linker as the 5′ connector exhibit higher RNA interfering activity (see e.g., Examples 1-2). The nucleic acid complex can have a modified nucleotide or a nucleotide as the 5′ connector. The nucleic acid complex can have no 5′ connector. The nucleic acid complex can have a C₃ 3-carbon linker, a modified nucleotide, or a nucleotide as the 3′ connector. The nucleic acid complex can have no 3′ connector. In some embodiments, not having a C₃ 3-carbon linker as the 5′ connector increases RNA interfering activity of the nucleic acid complex by at least about 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these value, greater than nucleic acid complexes having a C₃ 3-carbon linker as the 5′ connector.

In some embodiments, a nucleic acid complex having a C₃ 3-carbon linker as the 3′ connector exhibit higher RNA interfering activity (see e.g., Examples 1-2). The nucleic acid complex can have a modified nucleotide or a nucleotide as the 5′ connector. The nucleic acid complex can have no 5′ connector. The nucleic acid complex does not have a C₃ 3-carbon linker as the 5′ connector. In some embodiments, having a C₃ 3-carbon linker as the 3′ connector increases RNA interfering activity of the nucleic acid complex by at least about 2-fold, 3-fold, 4-fold, 5-fold, 6-fold, 7-fold, 8-fold, 9-fold, 10-fold, 15-fold, 20-fold, or a number or a range between any of these value, greater than nucleic acid complexes having a modified nucleotide (e.g. 2′-O-methyl nucleotide) as the 3′ connector. In some embodiments, having a C₃ 3-carbon linker as the 3′ connector increases RNA interfering activity of the nucleic acid complex by at least about 2-fold, 3-fold, 4-fold, 5-fold, 6-fold, 7-fold, 8-fold, 9-fold, 10-fold, 15-fold, 20-fold, or a number or a range between any of these value, greater than nucleic acid complexes having no 3′ connector.

In some embodiments, the core nucleic acid strand does not comprise a 5′ connector and/or a 3′ connector. Instead, each of the regions of the core nucleic acid strand is linked to its adjacent region via a standard phosphodiester linkage. In some embodiments, the central region of the core nucleic acid strand is linked to the 5′ region of the core nucleic acid strand via a phosphodiester linkage. In some embodiments, the central region of the core nucleic acid strand is linked to the 3′ region of the core nucleic acid strand via a phosphodiester linkage. In some embodiments, the central region of the core nucleic acid strand is linked to the 3′ region of the core nucleic acid strand via a phosphodiester linkage, while the central region of the core nucleic acid strand is linked to the 5′ region of the core nucleic acid strand via a 2′-O-methyl nucleotide such as 2′-O-methyladenosine, 2′-O-methylguanosine, 2′-O-methyluridine, or 2′-O-methylcytidine. In some embodiments, the central region of the core nucleic acid strand is linked to the 5′ region of the core nucleic acid strand via a phosphodiester linkage, while the central region of the core nucleic acid strand is linked to the 3′ region of the core nucleic acid strand via a 2′-O-methyl nucleotide such as 2′-O-methyladenosine, 2′-O-methylguanosine, 2′-O-methyluridine, or 2′-O-methylcytidine. In some embodiments, the central region of the core nucleic acid strand is linked to the 3′ region and the 5′ region of the core nucleic acid strand both via a phosphodiester linkage.

In some embodiments, not having a 5′ connector and/or a 3′ connector increases RNA interfering activity of the nucleic acid complex by at least about 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold or a number or a range between any of these value, greater than nucleic acid complexes having a C₃ 3-carbon linker as the 5′ connector.

In some embodiments, the core nucleic acid strand has an overhang (see e.g., Design 3 in FIGS. 5-6). The overhang can be at the 3′ terminus of the core nucleic acid strand (3′ overhang). In some embodiments, the core nucleic acid strand can have a short overhang at the 3′ terminus (e.g. 1-3 nucleosides), but the 3′ overhang does not extend back into the middle of the sensor duplex to bind with the sensor nucleic acid strand. The length of the overhang can vary in different embodiments. In some embodiments, the 3′ overhang is about one to three nucleotides in length. For example, the 3′ overhang can be one, two or three nucleotides in length. The overhang can comprise one or more modified nucleotides, such as 2′-O-methyl nucleotides. For example, the 3′ overhang can comprise two 2′-O-methyl nucleotides (see e.g., Design 3 shown in FIGS. 5-6). The overhang can comprise modified internucleoside linkages, such as phosphorothioate internucleoside linkages. In some embodiments, all of the nucleotides in the overhang are chemically modified. In some embodiments, all of internucleoside linkages in the 3′ overhang of the core nucleic acid strand are phosphorothioate internucleoside linkages.

The core nucleic acid strand can be designed from a passenger nucleic acid strand and a sensor nucleic acid strand. Methods and examples of designing a core nucleic acid strand from a passenger nucleic acid strand and a sensor nucleic acid strand are described, for example, in the related U.S. provisional application concurrently filed on Jul. 6, 2021 and entitled “Methods Of Generating Core Strands In Conditionally Activatable Nucleic Acid Complexes”, the content of which is incorporated by reference in its entirety.

### Passenger Nucleic Acid Strand

The passenger nucleic acid strand of the nucleic acid complex described herein is complementary bound to the central region or the first region of the core nucleic acid strand to form a RNAi duplex (e.g. a first nucleic acid duplex). Since the central region or the first region of the core nucleic acid strand is complementary to a target nucleic acid strand, the passenger nucleic strand of the nucleic acid complex can comprise a sequence homologous to the target nuclei acid strand.

As used herein, the term “homologous” or “homology” refers to sequence identity between at least two sequences. The term “sequence identity” or “identity” in the context of two nucleic acid or polypeptide sequences makes reference to the nucleotide bases or residues in the two sequences that are the same when aligned for maximum correspondence over a specified comparison window.

In some embodiments, the sequence identity between a passenger nucleic acid strand and a target nucleic acid or a portion there of can be, be about, be at least, or be at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values. The passenger nucleic acid strand of a nucleic acid complex can have a sequence substantially identical, e.g. at least 80%, 90%, or 100%, to a target nucleic acid or a portion thereof.

The length of the passenger nucleic acid strand can vary in different embodiments. In some embodiments, the passenger nucleic acid strand comprises 10-35 linked nucleosides. For example, the core nucleic acid strand can comprise 10, 11, 12, 13, 14, 15, 16, 17, 18, 19, 20, 21, 22, 23, 24, 25, 26, 27, 28, 29, 30, 31, 32, 33, 34, or 35 linked nucleosides. In some embodiments, the passenger nucleic acid strand comprises 17-21 linked nucleosides.

In some embodiments, the passenger nucleic acid strand has a 3′ overhang, a 5′ overhang, or both in the RNAi duplex. In some embodiments, the passenger nucleic acid strand has a 3′ overhang, and the 3′ overhang is one to five nucleosides in length.

In some embodiments, the overhang of the passenger nucleic acid strand is capable of binding to the input nucleic acid strand to form a toehold, thereby initiating a toehold mediated strand displacement and causing the displacement of the passenger nucleic acid strand from the core nucleic acid strand.

In some embodiments, the overhang of the passenger nucleic acid strand is 5 to 20 nucleosides in length. For example, the overhang of the passenger nucleic acid strand can be 5, 6, 7, 8, 9, 10, 11, 12, 13, 14, 15, 16, 17, 18, 19, or 20 nucleosides in length. In some embodiments, the overhang of the passenger nucleic acid strand is 9 nucleosides in length.

In some embodiments, one or more internucleoside linages of the overhang of the passenger nucleic acid strand are phosphorothioate internucleoside linkage which can protect the overhang from degradation. In some embodiments, all internucleoside linages of the overhang of the passenger nucleic acid strand can be phosphorothioate internucleoside linkage.

In some embodiments, the passenger nucleic acid strand is fully complementary to the central region or the first region of the core nucleic acid strand, thereby forming no overhang at the 5′ and 3′ termini of the passenger nucleic acid strand in the RNAi duplex. Therefore, in some embodiments, the passenger nucleic acid strand does not have a 3′ overhang, a 5′ overhang, or both in the RNAi duplex. In some embodiments, having a blunt end with no overhang can render the passenger nucleic acid strand unfavorable for Dicer binding, thereby bypassing the Dicer-mediated cleavage.

In some embodiments, the passenger nucleic acid strand is attached to a terminal moiety and/or a blocking moiety. Any suitable terminal moiety described herein that is capable of blocking the passenger nucleic acid strand from interacting with a RNAi pathway enzyme (e.g. Dicer, RISC) can be used. The blocking moiety can include one or more suitable terminal linkers or modifications such as a blocker that can protect a single-stranded nucleic acid from nuclease degradation such as an exonuclease blocking moiety. Examples of suitable blocking moieties include, but are not limited to, a dye (e.g. fluorophore, Cy3, a dark quencher), inverted dT, a linker to link the oligonucleotide with another molecule or a particular surface (biotins, amino-modifiers, alkynes, thiol modifiers, azide, N-Hydroxysuccinimide, and cholesterol), a space (e.g. C3 spacer, Spacer 9, Spacer 18, dSpacer, tri-ethylene glycol spacer, hexa-ethylene glycol spacer), a fatty acid, one or more modified nucleotides (e.g. 2′-O-methyl, 2′-F, PS backbone connection, LNA, and/or 2′-4′ bridged base) or a combination thereof. In some embodiments, the 5′ terminus of the passenger nucleic acid is attached to an inverted-dT, a tri-ethylene-glycol, or a fluorophore. For example, a fluorophore can be attached to the 5′ terminus of the passenger nucleic acid strand via a phosphorothioate linkage.

### Sensor Nucleic Acid Strand

The sensor nucleic acid strand of the nucleic acid complex described herein comprises a region complementary bound to the core nucleic acid strand, also referred to as the central region of the sensor nucleic acid strand. For example, in some embodiments, the central region of the sensor nucleic acid strand complementarily binds to the 5′ region and 3′ region of the core nucleic acid strand (see e.g., FIG. 4). In some other embodiments, the central region of the sensor nucleic acid strand complementarily binds to the second region of the core nucleic acid strand (see e.g., Design 3 in FIGS. 5-6).

The length of the central region complementary bound to the core nucleic acid strand can vary in different embodiments. In some embodiments, the central region complementary bound to the core nucleic acid strand comprises 10-35 linked nucleosides, optionally 10-30 linked nucleosides. For example, the central region in the sensor nucleic strand complementary bound to the 5′ region and the 3′ region of the core nucleic acid strand can comprise 10, 11, 12, 13, 14, 15, 16, 17, 18, 19, 20, 21, 22, 23, 24, 25, 26, 27, 28, 29, 30, 31, 32, 33, 34, or 35 linked nucleosides. In some embodiments, the central region in the sensor nucleic acid strand complementary bound to the core nucleic acid strand comprise about 14 linked nucleosides.

The sensor nucleic acid strand can comprise a toehold or an overhang. The overhang can be at the 3′ end or 5′ end, or both, of the sensor nucleic acid strand. The overhang is not complementary to the core nucleic acid strand and is capable of binding to an input nucleic acid strand, thereby initiating a toehold mediated strand displacement and causing the displacement of the passenger nucleic acid strand from the core nucleic acid strand. In some embodiments, the region of the sensor nucleic acid strand capable of binding an input nucleic acid strand covers the toehold region or a portion thereof and extends past the mid-point of the central region of the sensor stand.

The length of the overhang in the sensor nucleic acid strand can vary in different embodiments. In some embodiments, the length of the overhang can be 5-20 linked nucleotides. For example, the length of the overhang in the sensor nucleic acid strand can comprise 5, 6, 7, 8, 9, 10, 11, 12, 13, 14, 15, 16, 17, 18, 19, or 20 nucleotides in length. In some embodiments, the overhang of the sensor nucleic acid strand is 12 nucleotides in length. In some embodiments, the overhang of the sensor nucleic acid strand is 9 nucleotides in length.

The overhang of the sensor nucleic acid strand can comprise nucleotide modification introduced to improve the base-pairing affinity, nuclease resistance of the singled-stranded overhang, and thermodynamic stability to avoid spurious exonuclease induced activation of the strand. Exemplary modifications include, but not limited to, 2′-O-methyl modification, 2′-Fluoro modifications, phosphorothioate internucleoside linkages, inclusions of LNA, and the like that are identifiable by a skilled person. In some embodiments, at least 50% of the internucleoside linkages in the overhang of the sensor nucleic acid strand are phosphorothioate internucleoside linkages. For example, at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or a number or a range between any two values, of the internucleoside linkages in the overhang of the sensor nucleic acid strand are phosphorothioate internucleoside linkages. In some embodiments, all internucleoside linkages in the overhang of the sensor nucleic acid strand are phosphorothioate internucleoside linkages.

In some embodiments, the 5′ terminus and/or the 3′ terminus of the sensor nucleic acid strand can comprise a terminal moiety. Any suitable terminal moiety described herein can be used. In some embodiments, the terminal moiety can include a tri- or hexa-ethylene glycol spacer, a C3 spacer, an inverted dT, an amine linker, a ligand (e.g. a delivery ligand), a fluorophore, an exonuclease, a fatty acid, a Cy3, an inverted dT attached to a tri-ethylene glycol, or a combination thereof. In some embodiments, the 3′ terminus of the sensor nucleic acid strand can be attached to a delivery ligand, a dye (e.g. fluorophore), or exonuclease. The 5′ terminus can be attached to a fatty acid, a dye (e.g. Cy3), an inverted dT, a tri-ethylene glycol, or an inverted dT attached to a tri-ethylene glycol. The delivery ligand attached to the 3′ terminus can be any suitable ligand for use in targeting the nucleic acid complex to specific cell types described elsewhere in the present disclosure.

The sequence of the sensor nucleic acid strand can be designed to sense an input nucleic acid strand or a portion thereof. For example, from the sequence of an input biomarker, a list of all possible sensor segments which are antisense to the input strand can be generated. The sensor sequences for uniqueness in the transcriptome of the target animal can be ranked using NCBI BLAST. For human cancer cell lines, sequences can be checked against human transcript and genomic collection using the BLASTn algorithm. In some embodiments, sensor segments that have more than 17 bases of sequence complementarity and complete overhang complementarity to known or predicted RNA transcripts may be eliminated. Methods and examples of designing a sensor nucleic acid strand of a nucleic acid complex disclosed herein are described, for example, in the related application concurrently filed on Jul. 6, 2021 and entitled “Methods Of Designing Conditional-Activatable Small Interfering RNA Sensors”, the content of which is incorporated by reference in its entirety. The designed sensor nucleic acid can be further modified using the method herein described to introduce LNAs.

### Input Nucleic Acid Strand

The input nucleic acid strand described herein acts as a trigger to activate (switch on) the RNA interfering activity of the nucleic acid complex (e.g. RNAi duplex) upon binding to a sequence of the sensor nucleic acid in the nucleic acid complex. Therefore, the input nucleic acid strand comprises a mRNA of a gene or a variant thereof used in the method described herein to design the sensor nucleic acid.

The input nucleic acid strand comprises a sequence complementary to a sequence in the sensor nucleic acid strand of the nucleic acid complex. For example, the input nucleic acid strand can complementarily bind to a toehold (e.g. 3′ toehold) of the sensor nucleic acid strand. In some embodiments, the binding of the input nucleic acid strand initiates at the sensor toehold region and extends past the mid-point of the sensor duplex formed by the sensor nucleic acid strand and the core nucleic acid strand. The complementary binding between the input nucleic acid strand and the sensor nucleic acid strand causes displacement of the sensor nucleic acid strand from the core nucleic acid strand, thereby activating the RNA interfering activity of the RNAi duplex formed by the passenger nucleic acid strand and the central region of the core nucleic acid strand.

The input nucleic acid strand can be cellular RNA transcripts that are present at relatively high expression levels in a set of target cells (e.g. cancer cells) and at a relatively low level of expression in a set of non-target cells (e.g. normal cells). In some embodiments, the nucleic acid complex herein described is activated (switched on) in target cells. While in the non-target cells, the nucleic acid complex remains inactivated (switched off).

In the target cells, the input nucleic acid strand can be expressed at a level of, about, at least, or at least about 2-fold, 5-fold, 10-fold, 20-fold, 30-fold, 40-fold, 50-fold, 60-fold, 70-fold, 80-fold, 90-fold, or 100-fold higher than in the non-target cells.

In the target cells, the input nucleic acid strand can be expressed at a level of, about, at least, at least about 50, 100, 200, 300, 400, 500, 600, 700, 800, 900, 1000 transcripts. In some embodiments, in the non-target cells, the input nucleic acid strand is expressed at a level of less than 50, less than 40, less than 30, less than 20, or less than 10 transcripts. Preferably, the non-target cells have no detectable expression of the input nucleic acid strand.

The input nucleic acid strand can comprise an mRNA, an miRNA, or a non-coding RNA such as a long non-coding RNA, an RNA fragment, or an RNA transcript of a virus. In some embodiments, the input nucleic acid strand is an RNA transcript that is expressed in a set of cells that are causing the progression of a disease and are therefore targeted for RNAi therapy. The non-target cells are usually a set of cells where silencing of a target RNA can cause side effects that are not beneficial for therapy. For treating a disease or a condition where the input RNA is overexpressed in target cells, the nucleic acid complex can be designed such that the sensor nucleic acid strand comprises a sequence complementary to the input RNA sequence. Upon administration of the nucleic acid complex, the binding of sensor nucleic acid strand to the input RNA induces the dissociation of the RNAi duplex from the sensor duplex in target cells thereby to activate the RNAi targeting the disease or condition.

In some embodiments, the input nucleic acid strand comprises a biomarker. The term“biomarker” refers to a nucleic acid sequence (DNA or RNA) that is an indicator of a disease or disorder, a susceptibility to a disease or disorder, and/or of response to therapeutic or other intervention. A biomarker can reflect an expression, function or regulation of a gene. The input nucleic acid strand can comprise any disease biomarker known in the art. In some embodiments, the input nucleic acid strand is a mRNA, for example a cell type or cell state specific mRNA. Examples of a cell type or cell-state specific mRNA include, but are not limited to, C3, GFAP, NPPA, CSF1R, SLC1A2, PLP1, and MBP mRNA. In some embodiments, the input nucleic acid is a microRNA (also known as miRNA), including but is not limited to, hsa-mir-23a-3p, hsa-mir-124-3p, and hsa-mir-29b-3p. In some embodiments, the input nucleic acid strand is a non-coding RNA, for example MALAT1 (metastasis associated lung adenocarcinoma transcript 1, also known as NEAT2 (noncoding nuclear-enriched abundant transcript 2).

### Target RNA

The core nucleic acid strand (e.g. the central region or the second region) comprises a sequence complementary to a target RNA in order to direct target-specific RNA interference. The target RNA can be an mRNA, an miRNA, a non-coding RNA, a viral RNA transcript, a cellular RNA transcript, or a combination thereof.

As used herein, a “target RNA” refers to a RNA whose expression is to be selectively inhibited or silenced through RNA interference. A target RNA can be a target gene comprising any cellular gene or gene fragment whose expression or activity is associated with a disease, a disorder or a condition. A target RNA can also be a foreign or exogenous RNA or RNA fragment whose expression or activity is associated with a disease, a disorder or a certain condition (e.g. a viral RNA transcript or a pro-viral gene).

The target RNA can comprise an oncogene, a cytokinin gene, an idiotype protein gene (Id protein gene), a prion gene, a gene that expresses a protein that induces angiogenesis, an adhesion molecule, a cell surface receptor, a gene of a protein involved in a metastasizing and/or invasive process, a gene of a proteinase, a gene of a protein that regulates apoptosis and the cell cycle, a gene that expresses the EGF receptor, a multi-drug resistance 1 gene (MDR1), a gene of a human papilloma virus, a hepatitis C virus, or a human immunodeficiency virus, a gene involved in cardiac hypertrophy, or a fragment thereof.

The target RNA can comprise a gene encoding for a protein involved in apoptosis. Exemplary target RNA genes include, but are not limited to, bcl-2, p53, caspases, cytotoxic cytokines such as TNF-α or Fas ligand, and a number of other genes known in the art as capable of mediating apoptosis. The target RNA can comprise a gene involved in cell growth. Exemplary target RNA genes include, but not limited to, oncogenes (e.g., genes encoding for ABLI, BCLI, BCL2, BCL6, CBFA2, CBL, CSFIR, ERBA, ERBB, EBRB2, ETSI, ETSI, ETV6, FGR, FOS, FYN, HCR, HRAS, JUN, KRAS, LCK, LYN, MDM2, MLL, MYB, MYC, MYCLI, MYCN, NRAS, PIM I, PML, RET, SRC, TALI, TCL3, and YES), as well as genes encoding for tumor suppressor proteins (e.g., APC, BRCA1, BRCA2, MADH4, MCC, NF I, NF2, RB I, TP53, and WTI).

The target RNA can comprise a human major histocompatibility complex (MHC) gene or a fragment thereof. Exemplary MHC genes include MHC class I genes such as genes in the HLA-A, HLA-B or HLA-C subregions for class I cc chain genes, or β₂-microglobulinand and MHC class II genes such as any of the genes of the DP, DQ and DR subregions of class II α chain and β chain genes (i.e. DPα, DPβ, DQα, DQβ, DRα, and DRβ).

In some embodiments, the target RNA can comprise a gene encoding for a pathogen-associated protein. Pathogen associated protein include, but are not limited to, a viral protein involved in immunosuppression of the host, replication of the pathogen, transmission of the pathogen, or maintenance of the infection, or a host protein which facilitates entry of the pathogen into the host, drug metabolism by the pathogen or host, replication or integration of the pathogen's genome, establishment or spread of infection in the host, or assembly of the next generation of pathogen. In some embodiments, the pathogen can be a virus, such as a herpesvirus (e.g., herpes simplex, varicella-zoster virus, Epstein-Barr virus, cytomegalovirus (CMV)), hepatitis C, HIV, JC virus), a bacteria or a yeast.

In some embodiments, the target RNA comprises a gene associated with a disease or a condition of the central nervous system (CNS). Exemplary genes associated with a CNS disease or a condition include, but are not limited to, APP, MAPT, SOD1, BACE1, CASP3, TGM2, NFE2L3, TARDBP, ADRB1, CAMK2A, CBLN1, CDK5R1, GABRA1, MAPK10, NOS1, NPTX2, NRGN, NTS, PDCD2, PDE4D, PENK, SYT1, TTR, FUS, LRDD, CYBA, ATF3, ATF6, CASP2, CASP1, CASP7, CASP8, CASP9, HRK, C1QBP, BNIP3, MAPK8, MAPK14, Rac1, GSK3B, P2RX7, TRPM2, PARG, CD38, STEAP4, BMP2, GJA1, TYROBP, CTGF, ANXA2, RHOA, DUOX1, RTP801, RTP801L, NOX4, NOX1, NOX2 (gp91pho, CYBB), NOX5, DUOX2, NOXO1, NOXO2 (p47phox, NCF1), NOXA1, NOXA2 (p67phox, NCF2), p53 (TP53), HTRA2, KEAP1, SHC1, ZNHIT1, LGALS3, HI95, SOX9, ASPP1, ASPP2, CTSD, CAPNS1, FAS and FASLG, NOGO and NOGO-R; TLR1, TLR2, TLR3, TLR4, TLR6, TLR7, TLR8, TLR9, IL1bR, MYD88, TICAM, TIRAP, and HSP47.

### Executive Environment

FIG. 2 depicts a general architecture of an example computing device 200 configured to implement the method of placing LNAs in a nucleic acid strand of a conditionally activatable siRNA complex disclosed herein. The general architecture of the computing device 200 depicted in FIG. 2 includes an arrangement of computer hardware and software components. The computing device 200 may include many more (or fewer) elements than those shown in FIG. 2. It is not necessary, however, that all of these generally conventional elements be shown in order to provide an enabling disclosure. As illustrated, the computing device 200 includes a processing unit 210, a network interface 220, a computer readable medium drive 230, an input/output device interface 240, a display 250, and an input device 260, all of which may communicate with one another by way of a communication bus. The network interface 220 may provide connectivity to one or more networks or computing systems. The network interface 220 may also provide connectivity to one or more public databases to retrieve sequences and related information. The processing unit 210 may thus receive information and instructions from other computing systems or services via a network. The processing unit 210 may also communicate to and from memory 270 and further provide output information for an optional display 250 via the input/output device interface 240. The input/output device interface 240 may also accept input from the optional input device 260, such as a keyboard, mouse, digital pen, microphone, touch screen, gesture recognition system, voice recognition system, gamepad, accelerometer, gyroscope, or other input device.

The memory 270 may contain computer program instructions (grouped as modules or components in some embodiments) that the processing unit 210 executes in order to implement one or more embodiments. The memory 270 generally includes RAM, ROM and/or other persistent, auxiliary or non-transitory computer-readable media. The memory 270 may store an operating system 272 that provides computer program instructions for use by the processing unit 210 in the general administration and operation of the computing device 200. The memory 270 may further include computer program instructions and other information for implementing aspects of the present disclosure.

For example, in one embodiment, the memory 270 includes a LNA placement module 274 for placing LNAs in a nucleic acid strand, such as the method 100 for placing LNAs in a nucleic acid strand of a conditionally activable siRNA complex described with reference to FIG. 1. In addition, memory 270 may include or communicate with the data store 290 and/or one or more other data stores that store sequences, structures, parameters, and/or other information related to the method of placing LNAs in the nucleic acid strand and the generated LNA-modified nucleic acid strand. The one or more data stores can also store the information generated during the process including, for example, the sequence of the nucleic acid strand, the secondary structures of the nucleic acid strand, diagrams and/or graphs representing the secondary structures, the plurality of sequence regions identified, the plurality of sequence regions modified with LNAs, and other data generated by the method described herein.

The methods described herein have been used and can be used to design, for example, the conditional activatable nucleic acid complexes described in the related U.S. Provisional Application No. 63/172,030 filed on Apr. 7, 2021 as “Using siRNA To Treat Neurodegenerative Diseases” and U.S. provisional application concurrently filed on Jul. 6, 2021 and entitled “Conditionally Activatable Nucleic Acid Complexes,” the content of each of these related applications is incorporated by reference in its entirety.

### EXAMPLES

Some aspects of the embodiments discussed above are disclosed in further detail in the following examples, which are not in any way intended to limit the scope of the present disclosure.

### Example 1. RNAi Activity with or without a C3 Linker

This example demonstrates the RNAi activity of various siRNA domain variants with or without a C3 linker as the 5′ and the 3′ connector.

The passenger and core strands of the new construct are assembled to form the siRNA domains of the new construct. The different variants of these siRNA domains are tested for RNAi activity.

To test the constructs, CASi siRNA segments were assembled by thermally annealing passenger and core strands in 1× phosphate buffer saline. The RNAi activities of the CASi siRNA segments were measured using dual luciferase assays. CASi siRNA segments were co-transfected into HCT 116 cells with dual luciferase vectors carrying the Huntingtin gene siRNA target sequence, using lipofectamine 2000. After 48 hours, cells were lysed and assayed for knockdown of the target gene by comparing the luminescence value of Renilla luciferase that carries the target sequence to Firefly luciferse that was used as a reference control. Methods and procedures of assembling CASi siRNA, cell transfection, and dual luciferase assays can be found in, for example, international application WO 2020/033938, the content of which is incorporated herein by reference in its entirety.

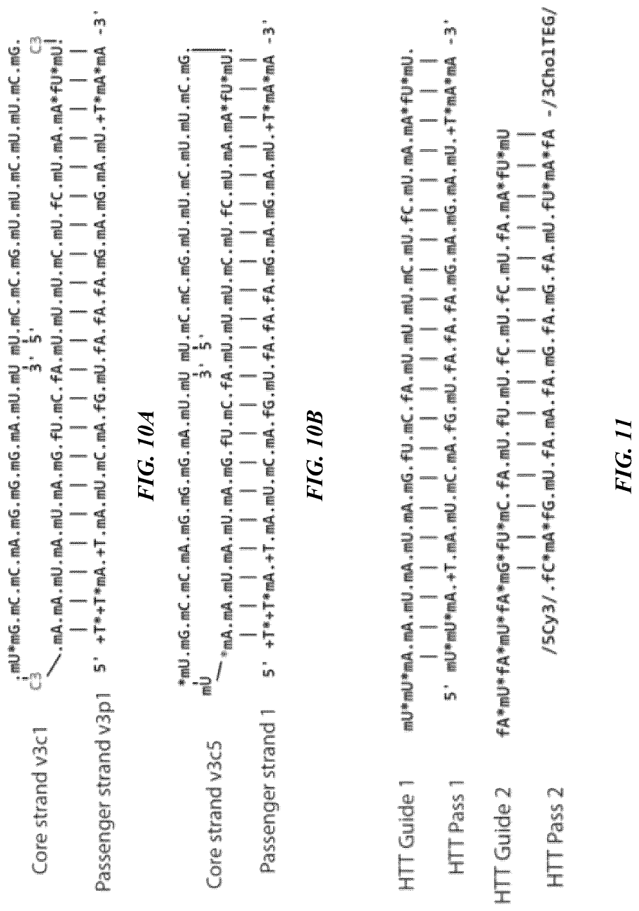

FIG. 10A and FIG. 10B show sequence diagrams of two exemplary nucleic acid complex constructs whose RNAi activities are determined in this example. Top nucleic acid complex construct comprises a core strand v3c1 base-paired to a passenger strand v3p1, in which a C3 linker is used as the 5′ and the 3′ connector. Bottom nucleic acid complex construct comprises a core strand v3c5 base-paired to the same passenger strand, in which no C3 linker is used as the 5′ and the 3′ connector. Instead, v3c5 core strand has a 3′ mU connector and no connector at the 5′ end.

FIG. 11 shows sequence diagrams of two positive control nucleic acid complex constructs designed to target Huntingtin gene (HTT gene) used in the assay described in this example.

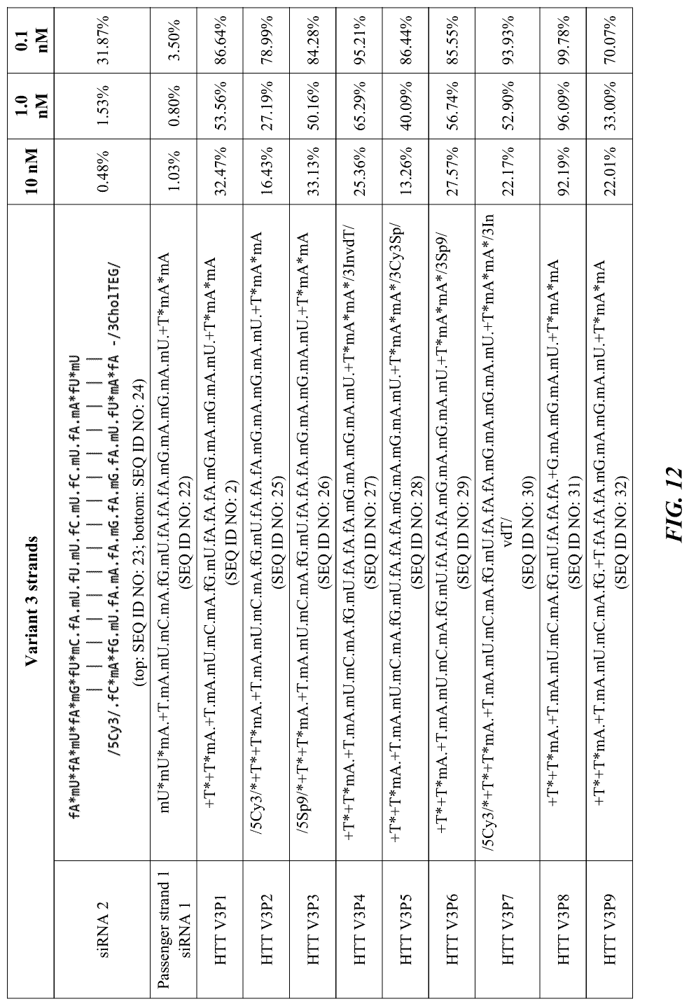

FIG. 12 shows various siRNA variants with different passenger strand (V3P1, V3P2, V3P3, V3P5, V3P5, V3P6, V3P7, V3P8, and V3P9) assembled with v3c1 core strand shown in FIG. 10A and tested in this example. The v3c1 core strand has a C3 linker as the 5′ and the 3′ connector. The target protein expression was tested with the siRNA variants at three different concentrations: 10 nM, 1.0 nM, and 0.1 nM.

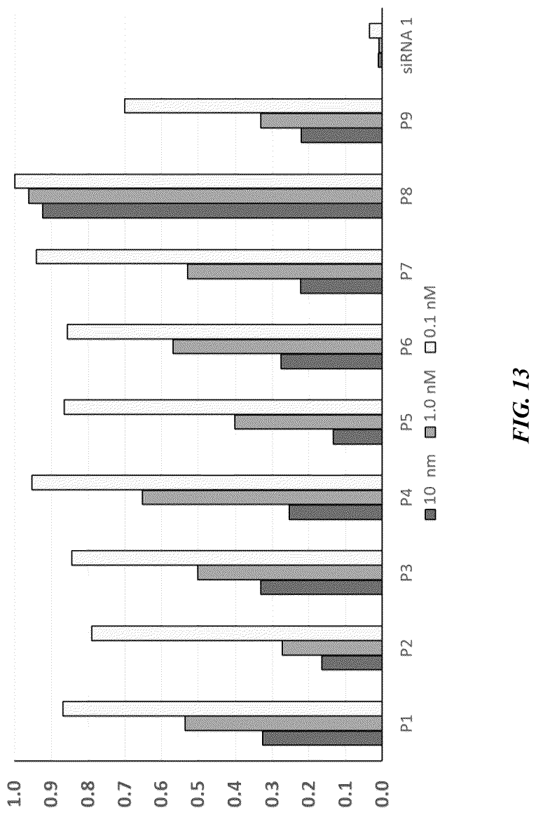

FIG. 13 shows a graphic representation of the target protein expression data for the siRNA variants shown in FIG. 12. Higher RNAi activity is suggested by lower expression of the target protein.

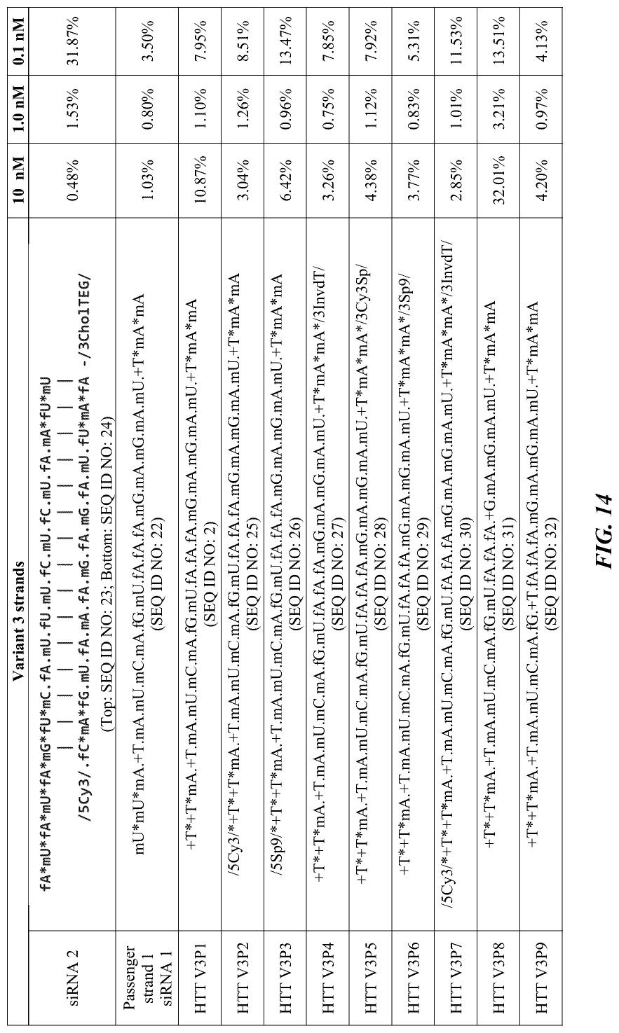

FIG. 14 shows different siRNA variants with different passenger strand (V3P1, V3P2, V3P3, V3P5, V3P5, V3P6, V3P7, V3P8, and V3P9) assembled with a v3c5 core strand shown in FIG. 10B and tested in this example. The v3c5 core strand does not have a C3 linker as the 5′ and the 3′ connector. Instead, v3c5 core strand has a 3′ mU connector and no connector at the 5′ end. The target protein expression was tested with the siRNA variants at three different concentrations: 10 nM, 1.0 nM, and 0.1 nM.

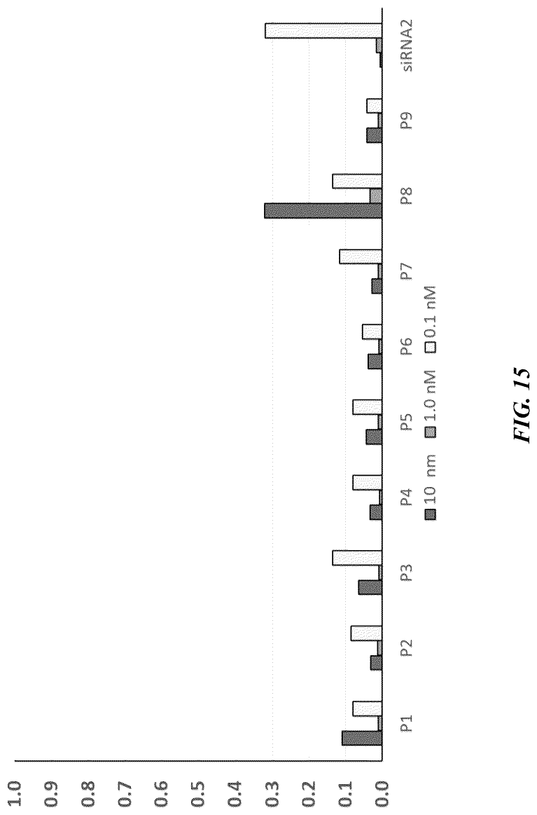

FIG. 15 shows a graphic representation of the target protein expression data for the siRNA variants shown in FIG. 14. Similar to FIGS. 12-13, higher RNAi activity is suggested by lower expression of the target protein.

These data indicate that a C3 linker as the 5′ connector inhibits RNAi activity of the siRNA domain. A comparison of the target protein expression data among different passenger variants (V3P1, V3P2, V3P3, V3P5, V3P5, V3P6, V3P7, V3P8, and V3P9) indicates that extensive modification of the passenger strand with LNAs (e.g. HTT V3P8) can decrease RNAi activity.

### Example 2. RNAi Activity with Different 5′ and 3′ Connectors

In this example, different versions of the core strand were tested with the same sensor (Mir23 Sensor 1) and passenger strands (Passenger strand 1) to investigate the effects of different 5′ and 3′ connectors on the RNAi activity. RNAi activity was also evaluated between two-stranded constructs and three-stranded constructs.

Two-stranded constructs consist of the passenger strand base-paired to the core strand, forming an active siRNA domain. Three-stranded constructs consist of all three strands: the passenger strand, the core strand, and the sensor strand.

CASi siRNA segments (two-stranded constructs) and three-stranded constructs were assembled by thermally annealing passenger and core strands, or passenger, core and sensor strands in 1× phosphate buffer saline.

CASi siRNA segments or three-stranded constructs were co-transfected into HCT 116 cells with dual luciferase vectors carrying the Huntingtin gene siRNA target sequence, using lipofectamine 2000. After 48 hours, cells were lysed and assayed for knockdown of the target gene by comparing the luminescence value of Renilla luciferase that carries the target sequence to Firefly luciferse that was used as a reference control. Examples of methods and procedures of assembling CASi siRNA, cell transfection, and dual luciferase assays are described in, for example, international application WO 2020/033938.

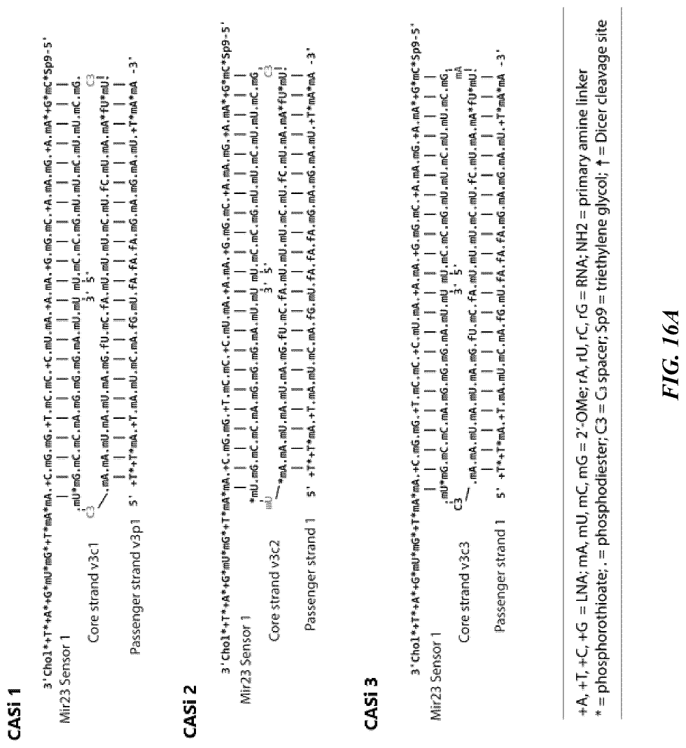

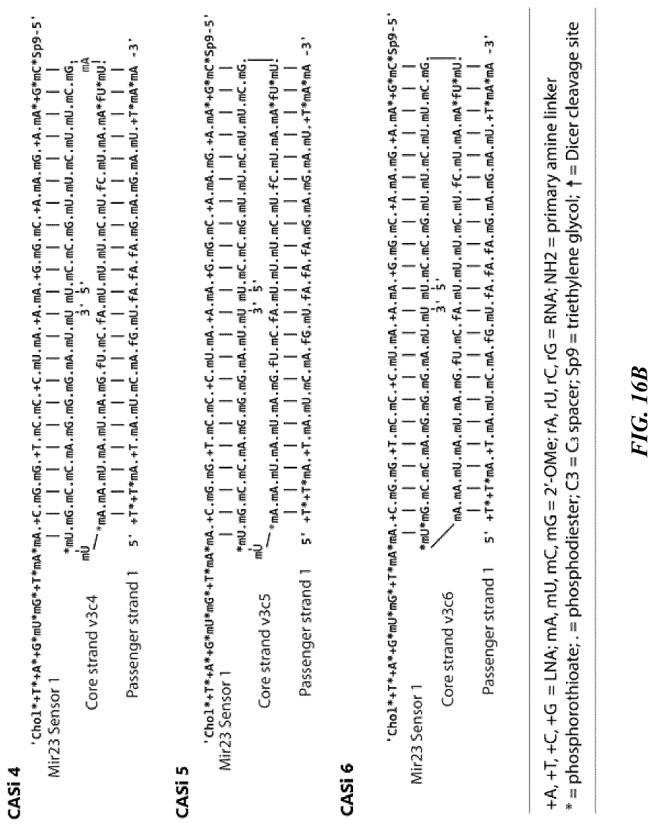

FIG. 16A and FIG. 16B show sequence diagrams of various nucleic acid complexes disclosed herein each having the same passenger strand (Passenger strand 1) and the sensor strand (Mir23 Sensor 1) but a different core strand (Core strand v3c1, Core strand v3c2, Core strand v3c3, Core strand v3c4, Core strand v3c5, and Core strand v3c6), and particularly, a different 5′ and 3′ connector in the core strand.

The sequences illustrated in FIGS. 16A and 16B are also provided in Table 2 below.

TABLE 2
Sequences of exemplary CASi strands.
mir23 sensor

| | |
|---|---|
| mir23 Sensor 1 | /5Sp9/*mC*+G*mA.+A.mG.mA.+A.mC.mG.+G.mA.+A.mA.mU |
| | .+C.mC.mC.+T.mG.mG.+C.mA*mA*+T*mG*mU*+G*+A*+T*/ |
| | 3CholTEG/ |
| | (SEQ ID NO: 1) |

Passenger strand

| | |
|---|---|
| HTT V3P1 | +T*+T*mA.+T.mA.mU.mC.mA.fG.mU.fA.fA.fA.mG.mA.mG. |
| | mA.mU.+T*mA*mA |
| | (SEQ ID NO: 2) |

Core strands

| | |
|---|---|
| CASi 1: V3C1 | mU.mC.mC.mG.mU.mU.mC.mU.mU.mC.mG./iSpC3/.mU*fU*m |
| | A.mA.mU.fC.mU.mC.mU.mU.mU.fA.mC.fU.mG.mA.mU.mA.m |
| | U.mA.mA./iSpC3/.mU*mG.mC.mC.mA.mG.mG.mG.mA.mU.mU |
| | (SEQ ID NO: 3, SEQ ID NO: 4, |
| | and SEQ ID NO: 5 joined by an |
| | internal C3 spacer “iSpC3”) |
| CASi 2: V3C2 | mU.mC.mC.mG.mU.mU.mC.mU.mU.mC.mG./iSpC3/.mU*fU*m |
| | A.mA.mU.fC.mU.mC.mU.mU.mU.fA.mC.fU.mG.mA.mU.mA.m |
| | U.mA.mA*mU*mU.mG.mC.mC.mA.mG.mG.mG.mA.mU.mU |
| | (SEQ ID NO: 6 and SEQ ID NO: 7 joined by iSpC3) |
| CASi 3: V3C3 | mU.mC.mC.mG.mU.mU.mC.mU.mU.mC.mG.mA.mU*fU*mA.mA. |
| | mU.fC.mU.mC.mU.mU.mU.fA.mC.fU.mG.mA.mU.mA.mU.mA. |
| | mA./iSpC3/.mU*mG.mC.mC.mA.mG.mG.mG.mA.mU.mU |
| | (SEQ ID NO: 8 and SEQ ID NO: 9 joined by iSpC3) |
| CASi 4: V3C4 | mU.mC.mC.mG.mU.mU.mC.mU.mU.mC.mG.mA.mU*fU*mA.mA. |
| | mU.fC.mU.mC.mU.mU.mU.fA.mC.fU.mG.mA.mU.mA.mU.mA. |
| | mA*mU*mU.mG.mC.mC.mA.mG.mG.mG.mA.mU.mU |
| | (SEQ ID NO: 10) |
| CASi 5: V3C5 | mU.mC.mC.mG.mU.mU.mC.mU.mU.mC.mG.mU*fU*mA.mA.mU. |
| | fC.mU.mC.mU.mU.mU.fA.mC.fU.mG.mA.mU.mA.mU.mA.mA* |
| | mU*mU.mG.mC.mC.mA.mG.mG.mG.mA.mU.mU |
| | (SEQ ID NO: 11) |
| CASi 6: V3C6 | mU.mC.mC.mG.mU.mU.mC.mU.mU.mC.mG.mU*fU*mA.mA.mU. |
| | fC.mU.mC.mU.mU.mU.fA.mC.fU.mG.mA.mU.mA.mU.mA.mA* |
| | mU*mG.mC.mC.mA.mG.mG.mG.mA.mU.mU |
| | (SEQ ID NO: 12) |

/Sp9/ = triethylene glycol spacer
CholTEG = Cholesterol-TEG
/iSpC3/ = internal C3 spacer
* = phosphorothioate backbone
. = phosphodiester backbone
mA, mG, mC, mU = 2′-O-methyl bases
+A, +T, +C, +G = locked nucleic acid (LNA) bases
fA, fU, fC, fG = 2′-fluoro bases
NH2 = primary amine linker.
rA, rU, rC, rG = RNA

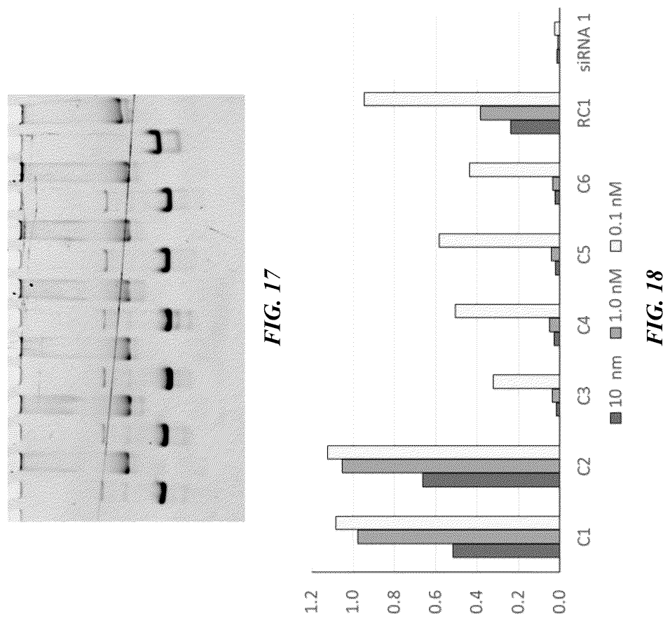

FIG. 17 shows non-denaturing polyacrylamide gel (PAGE) of various nucleic acid complex constructs, indicating all the complexes are assembled as desired. Lanes are as follows (from left to right): P1C1; P1C1S2; P1C2; P1C2S2; P1C3; P1C3S2; P1C4; P1C4S2; P1C5; P1C5S2; P1C6; P1C6S2; G1RC1; and G1RC1S2. P1 indicates the passenger strand 1.

FIG. 18 shows the RNAi activity of two-stranded assemblies each having the same passenger strand v3p1 and a different core strand (C1, C2, C3, C4, C5, and C6) at different concentrations. The sequences of the passenger strand and the core strand are shown in FIGS. 16A and 16B.

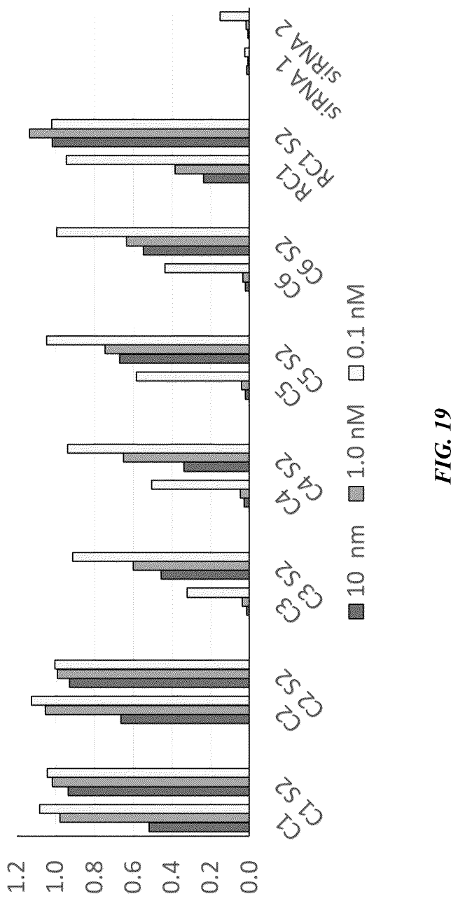

FIG. 19 shows the RNAi activity of three-stranded assemblies each having the same passenger strand v3p1, the same sensor strand (Mir23 sensor 1), and a different core strand (C1, C2, C3, C4, C5, and C6) at three different concentrations. The sequences of the passenger strand, the sensor strand, the core strand are shown in FIGS. 16A and 16B.

These data indicate that assemblies, including two-stranded and three-stranded assembles, with 5′ mA connector and 3′ C3 (3-carbon linker) connector has the highest RNAi activity. Assemblies, including two-stranded and three-stranded assembles, which do not have a 5′ C3 connector (such as C3, C4, C5, C6) have a higher RNAi activity than assemblies having a 5′ C3 connector (C1 and C2). Assemblies that do not have a 5′ connector (C5 and C6) have a lower RNAi activity than assemblies (C3 and C4) having a 5′ connector (such as mA) but not a C3 linker. For the same core strand, the three-stranded assemblies are generally expected to have lower RNAi activity than two-stranded assemblies.

### Example 3. RNAi Activity of Various RNA Complex Designs

In this example, experiments were carried out to compare the RNAi switching and RNAi activity of Design 1 shown in FIG. 4 and the RNA complex design disclosed herein (e.g., Design 2 shown in FIG. 4). V3C3a and V3C3b are the constructs in the form of Design 2. G1C1S1 is a construct in the form of the Design 1.

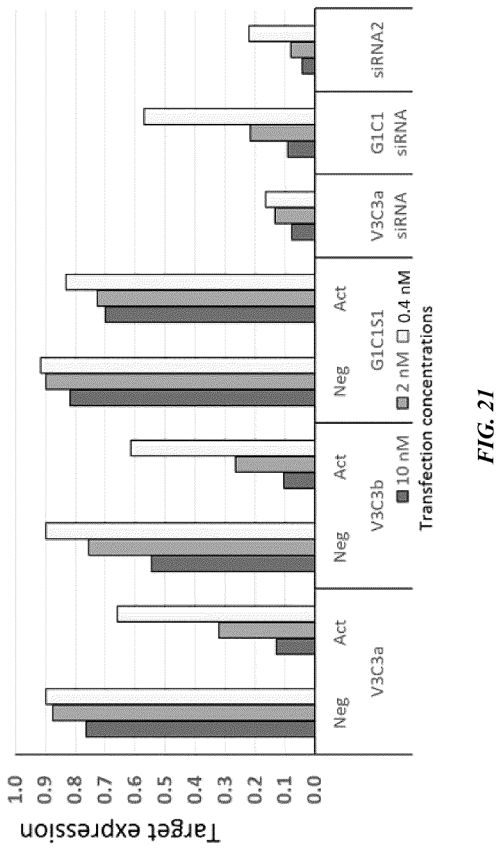

CASi siRNA segment (two-stranded constructs) and three-stranded constructs were assembled by thermally annealing passenger and core strands, or passenger, core and sensor strands in 1× phosphate buffer saline. The CASi siRNA segment (two-stranded constructs) and three-stranded constructs were co-transfected into HCT 116 cells using lipofectamine 2000. The HCT116 cells can express either an RNA biomarker that could activate the CASi sensor (e.g. NPPA gene sequence encoding atrial natriuretic peptide (ANP)) (denoted as “Act” in FIG. 21) or a control nucleic acid strand that could not activate the CASi sensor (denoted as “Neg” in FIG. 21) using a short RNA transcript driven by a Pol III promoter. The HCT 116 cells also have a dual luciferase vector carrying the calcineurin gene siRNA target sequence. Calcineurin is a calcium and calmodulin dependent serine/threonine protein phosphatase, and has been identified as a key driver of cardiac hypertrophy. ANP has been used as diagnostic markers for cardiac hypertrophy. Therefore, the sensor strand of the three-stranded CASi siRNA constructs is designed to detect ANP mRNA while the siRNA domain (e.g. the passenger strand) is designed to inhibit calcineurin.

After 72 hours, cells were lysed and assayed for knockdown of the target gene (calcineurin) by comparing the luminescence value of Renilla luciferase (carrying the target sequence) to Firefly luciferase.

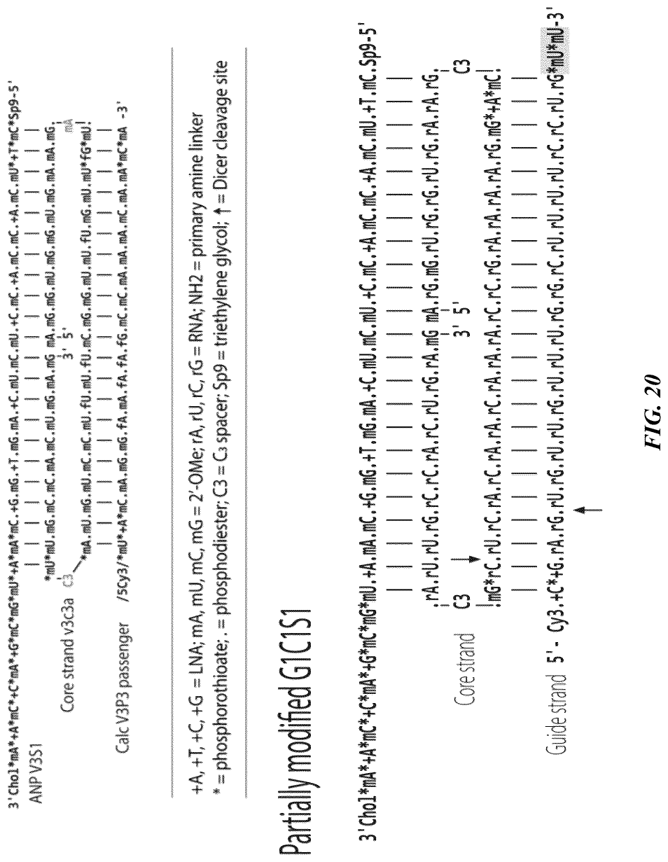

FIG. 20 shows sequence diagrams of a nuclei acid complex including a core strand V3C3a in the form of Design 2 (T2 CASi) shown in FIG. 4 and a nucleic acid complex in the form of Design 1 (Cond-siRNA construct) shown in FIG. 4 (bottom: G1C1S1). The sequences of T2 CASi and Cond-siRNA strands are provided in Table 3.

TABLE 3
Sequences of T2 CASi of Design 2 and an Cond-siRNA of Design 1.
Passenger for T2 ANP-calcineurin CASi

| | |
|---|---|
| Calc V3P3 | /5Cy3/*mU*+A*mC.mA.mG.mG.fA.mA.fA.fA.fG.mC.mC.mA |
| passenger | .mA.mA.mC.mA.mA*mC*mA |
| | (SEQ ID NO: 13) |

Sensor strand for both T2 CASi and Cond-siRNA

| | |
|---|---|
| ANP Sensor 1 | /5Sp9/.mC.+T.mU.mC.+A.mC.mC.+A.mC.+C.mU.mC.mU.+C |
| | .mA.mG.+T.mG.+G.mC.mA.+A.mU*mG*mC*+G*mA*+C*mC*+A |
| | *mA* / 3TEGChol/ |
| | (SEQ ID NO: 14) |

Core strand for T2 ANP-calcineurin CASi

| | |
|---|---|
| Rat ANP V3C3a | mA.mG.mG.mU.mG.mG.mU.mG.mA.mA.mG.mA.mU*fG*mU.mU. |
| | mG.fU.mU.mU.mG.mG.mC.fU.mU.fU.mU.mC.mC.mU.mG.mU. |
| | mA*/iSpC3/*mU*mU.mG.mC.mC.mA.mC.mU.mG.mA.mG |
| | (SEQ ID NO: 15 and SEQ ID NO: 16 joined by iSpC3) |

PPP3CA guide strand for Cond-siRNA

| | |
|---|---|
| Calc G4 | /5Cy3/.+C*+G.rA.rG.rU.rG.rU.rU.rG.rU.mU.rU.mG.mG |
| | .mC.rU.mU.rU.rU.rC.mC.mU.mG*mU*mU |
| | (SEQ ID NO: 17) |

Core strand for Cond-siRNA

| | |
|---|---|
| ANP-Calc core | mA.mG.mG.mU.mG.mG.mU.mG.mA.mA.mG./iSpC3/.mC*+A*m |
| strand | G.rG.rA.rA.rA.rA.rG.rC.rC.rA.rA.rA.rC.rA.rA.rC.r |
| | A.rC.rU.rC*mG./iSpC3/.mA.mU.mU.mG.mC.mC.mA.mU.mU |
| | .mG.mA.mG |
| | (SEQ ID NO: 18, SEQ ID NO: 19 and SEQ ID NO: 20 |
| | joined by iSpC3) |

/Sp9/ = triethylene glycol spacer
CholTEG = Cholesterol-TEG
/iSpC3/ = internal C3 spacer
* = phosphorothioate backbone
. = phosphodiester backbone
mA, mG, mC, mU = 2′-O-methyl bases
+A, +T, +C, +G = locked nucleic acid (LNA) bases
fA, fU, fC, fG = 2′-fluoro bases
NH2 = primary amine linker.
rA, rU, rC, rG = RNA

FIG. 21 shows the RNAi activity of the modified two-stranded constructs (V3C3a siRNA) and three-stranded constructs (V3C3a and V3C3b) in comparison with the original two-stranded (G1C1 siRNA) and three-stranded constructs (G1C1S1) at three different concentrations.

These data indicate that the modified CASi constructs shows lower RNAi activity in the absence of the RNA biomarker (Neg) and higher RNAi activity in the presence of the RNAi biomarker (Act), thus indicating that the RNAi activity of the modified CASi constructs is switched OFF when the RNA biomarker is absent. The RNAi activity of the modified constructs (V3C3a and V3C3b) was also significantly improved compared to the original design (G1C1S1). The modified CASi siRNA segments (two-stranded assemblies, e.g. V3C3a siRNA)) also show significantly improved RNAi activity compared to the original two-stranded design (G1C1 siRNA).

### Example 4. Determination of RNAi Activity

This example describes performing RNAi activity of various nucleic acid complex constructs described herein.

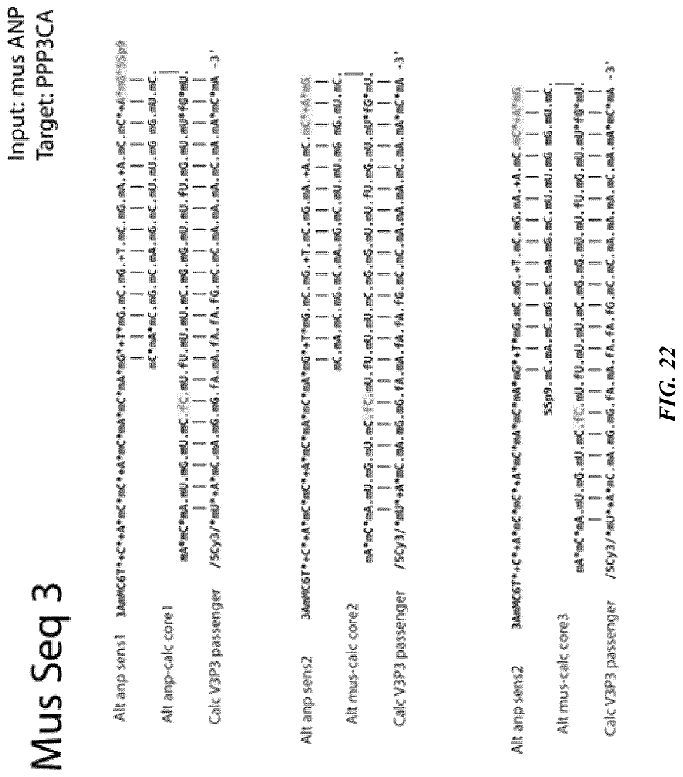

Different variants of the CASi siRNA constructs shown in FIG. 22 can be tested for RNAi activity. The sensor strand of the constructs can be designed to sense an input nucleic acid, such as a NPPA gene sequence encoding atrial natriuretic peptide (ANP). To test the constructs, CASi siRNA constructs can be assembled by thermally annealing the passenger strand, the core strand and the sensor strand in 1× phosphate buffer saline. The RNAi activities of the CASi siRNA constructs can be measured using dual luciferase assays. CASi siRNA constructs can be co-transfected into HCT 116 cells with dual luciferase vectors carrying a calcineurin gene target sequence (PPP3A), using lipofectamine 2000. After 48 hours, cells can be lysed and assayed for knockdown of the target gene by comparing the luminescence value of Renilla luciferase that carries the target sequence to Firefly luciferase that can be used as a reference control. Examples of methods and procedures of assembling CASi siRNA constructs, cell transfection, and dual luciferase assays are described in, for example, international application WO/2020/033938, the content of which is incorporated herein by reference in its entirety. It is expected that the RNA complexes described herein have RNAi activities.

### Terminology

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method of introducing a plurality of locked nucleic acid (LNA) modifications in a nucleic acid strand, comprising:
under control of a hardware processor:
(a) determining a secondary structure of the nucleic acid strand;
(b) determining if each nucleotide of the nucleic acid strand is capable of base pairing with another nucleotide of the nucleic acid strand;
(c) identifying a plurality of sequence regions in the nucleic acid strand, each having a starting position and an end position and each comprising a nucleotide capable of base pairing with at most one other nucleotide in the secondary structure;
(d) for each of the plurality of sequence region identified in (c), from the starting position to the end position
(i) introducing a first LNA modification at a first nucleotide of the sequence region capable of forming a minimum number of base pairs with other nucleotides of the nucleic acid strand;
(ii) introducing a second LNA modification at a second nucleotide of the sequence region at least two bases downstream from the first LNA modification introduced if the second nucleotide is not a guanine (G); and
(iii) repeating (ii) until reaching the end position of the sequence region to generate a LNA-modified nucleic acid strand.

2. The method of claim 1, wherein the secondary structure of the nucleic acid strand comprises an internal secondary structure formed by the nucleic acid strand, a self-duplex secondary structure formed by two interacting nucleic acid strands, or both.

3. The method of claim 1 or 2, wherein the secondary structure of the nucleic acid strand has a minimal free energy.

4. The method of any one of claims 1-3, wherein obtaining the secondary structure of the nucleic acid strand comprises calculating a free energy of each of a plurality of secondary structures of the nucleic acid strand and identifying the secondary structure of the nucleic acid strand having a minimal free energy; and optionally calculating the free energy of each of the plurality of secondary structures of the nucleic acid strand comprises using a nearest neighbor model.

5. The method of any one of claims 1-4, wherein analyzing the secondary structure of the nucleic acid strand comprises: determining a base pair score for each nucleotide of the nucleic acid strand, the base pair score being proportional to the number of base pairs a nucleotide forms with one or more nucleotides of the nucleic acid strand; and optionally the base pairs are formed between nucleotides of a single nucleotide acid strand or between nucleotides of two interacting nucleotide acid strands.

6. The method of claim 5, wherein the number of base pairs the nucleotide forms with one or more nucleotides of the nucleic acid strand is at most three.

7. The method of any one of claims 5-6, wherein identifying the plurality of sequence regions each having a starting position and an end position and each comprising a nucleotide forming at most one base pair with another nucleotide in the secondary structure, comprises: identifying, for each sequence region, at least one nucleotide having a lowest base pair score.

8. The method of any one of claims 1-7, comprising eliminating any sequence region in which each nucleotide of the sequence region is capable of base pairing with two or more other nucleotides in the nucleic acid strand.

9. The method of any one of claims 1-8, wherein the plurality of sequence regions each have 2-20 nucleotides in length.

10. The method of any one of claims 1-9, wherein the first nucleotide of the sequence region at which the first LNA modification is introduced is not a G.

11. The method of any one of claims 1-10, comprising:
identifying one or more nucleotide of the sequence region each forming a minimum number of base pairs with other nucleotides of the nucleic acid strand.

12. The method of any one of claims 1-11, wherein the second nucleotide of the sequence region forms a minimum number of base pairs with other nucleotides of the nucleic acid strand.

13. The method of any one of claims 1-12, wherein introducing the first LNA modification at the first nucleotide of the sequence region forming the minimum number of base pairs with other nucleotides of the nucleic acid strand comprises:
identifying the first nucleotide of the sequence region that is not guanine (G) and that forms the minimum number of base pairs with other nucleotides of the nucleic acid strand; and
introducing the first LNA modification at the first nucleotide of the sequence region.

14. The method of any one of claims 1-13, comprising:
introducing the first LNA modification at the first nucleotide of the sequence region that is a G and that form a minimum number of base pairs with other nucleotides of the nucleic acid strand, if all the nucleotides of the sequence region each forming the minimum number of base pairs with other nucleotides of the nucleic acid strand are G.

15. The method of any one of claims 1-14, wherein the second LNA modification is introduced at the second nucleotide of the sequence region three bases downstream from the first LNA modification introduced.

16. The method of any one of claims 1-14, wherein the second LNA modification is introduced at the second nucleotide of the sequence region four bases downstream from the first LNA modification introduced.

17. The method of any one of claims 1-16, wherein the plurality of sequence regions does not overlap with one another when aligned with the nucleic acid strand.

18. The method of any one of claims 1-17, wherein the LNA modification comprises introducing a chemical bridge connecting the 2′ and 4′ carbons of a nucleotide; and optionally the chemical bridge is a 2′-O, 4′-C methylene bridge or a 2′-O, 4′-C ethylene bridge.

19. The method of any one of claims 1-18, wherein about 10%-50% of the nucleotides of the nucleic acid strand are modified with LNA or analogues thereof.

20. The method of any one of claims 1-19, wherein any two LNA modifications of the plurality of LNA modifications are at least one nucleotide apart; and optionally two LNA modifications of the plurality of LNA modifications are two nucleotides apart.

21. The method of any one of claims 1-20, wherein the 5′ terminus, the 3′ terminus, or both of the LNA-modified nucleic acid strand comprises a terminal moiety; and optionally the terminal moiety comprises a ligand, a fluorophore, a exonuclease, a fatty acid, a Cy3, an inverted dT attached to a tri-ethylene glycol, or a combination thereof.

22. The method of any one of claims 1-21, wherein the nucleotide acid strand has 10-35 nucleotides in length.

23. The method of any one of claims 1-22, further comprising producing the LNA-modified nucleic acid strand.

24. A method for producing a nucleic acid complex, comprising:
providing a first nucleic acid strand comprising 20-70 linked nucleosides;
providing a second nucleic acid strand;
providing a third nucleic acid strand, wherein one or more of the first, second and third nucleic acid strands is a LNA-modified nucleic acid strand produced by the method of claim 23; and
contacting the first nucleic acid strand, the second nucleic strand, and the third nucleic acid strand under a condition for a period of time to form a nucleic acid complex, wherein the nucleic acid complex comprises:
the second nucleic acid strand binding to a central region of the first nucleic acid strand to form a first nucleic acid duplex; and
the third nucleic acid strand binding to a 5′ region and a 3′ region of the first nucleic acid strand to form a second nucleic acid duplex, wherein the third nucleic acid strand comprises a 3′ toehold that is not complementary to the first nucleic acid strand and is capable of binding to an input nucleic acid strand to cause the displacement of the third nucleic acid strand from the first nucleic acid strand.

25. The method of claim 24, wherein the central region of the first nucleic acid strand comprises a sequence complementary to a target RNA.

26. The method of any one of claim 24 and 25, wherein the sequence complementary to a target RNA is 10-35 nucleosides in length.

27. A method for producing a nucleic acid complex, comprising:
providing a first nucleic acid strand comprising 20-70 linked nucleosides;
providing a second nucleic acid strand;
providing a third nucleic acid strand, wherein one or more of the first, second and third nucleic acid strands is a LNA-modified nucleic acid strand produced by the method of claim 23; and
contacting the first nucleic acid strand, the second nucleic strand, and the third nucleic acid strand under a condition for a period of time to form a nucleic acid complex, wherein the nucleic acid complex comprises:
the second nucleic acid strand binding to a first region of the first nucleic acid strand to form a first nucleic acid duplex; and
the third nucleic acid strand binding to a second region of the first nucleic acid strand to form a second nucleic acid duplex, wherein the third nucleic acid strand comprises a 3′ toehold that is not complementary to the first nucleic acid strand and is capable of binding to an input nucleic acid strand to cause the displacement of the third nucleic acid strand from the first nucleic acid strand, and
wherein
the first region of the first nucleic acid strand is 3′ of the second region of the first nucleic acid strand, and
the third nucleic acid strand does not bind to any region of the first nucleic acid strand that is 3′ of the first region of the first nucleic acid strand.

28. The method of claim 27, wherein the first region of the first nucleic acid strand comprises a sequence complementary to a target RNA.

29. The method of any one of claim 27 and 28, wherein the sequence complementary to a target RNA is 10-35 nucleosides in length wherein the sequence is 10-35 nucleosides in length.

30. The method of any one of claims 24-29, the third nucleic acid strand comprises a 5′ toehold.


SEQUENCE LISTING (37 sequences)

SEQ ID NO: 1 - RNA, 29 nt, synthetic construct
  source 1..29: mol_type = other RNA
  misc_binding 1: bound_moiety = triethylene glycol spacer
  misc_binding 29: bound_moiety = Cholesterol-TEG
  modified_base 1: mod_base = cm
  modified_base 2: mod_base = OTHER; note = locked nucleic acid base
  modified_base 3: mod_base = OTHER; note = 2'-O-methylated base
  modified_base 4: mod_base = OTHER; note = locked nucleic acid base
  modified_base 5: mod_base = gm
  modified_base 6: mod_base = OTHER; note = 2'-O-methylated base
  modified_base 7: mod_base = OTHER; note = locked nucleic acid base
  modified_base 8: mod_base = cm
  modified_base 9: mod_base = gm
  modified_base 10: mod_base = OTHER; note = locked nucleic acid base
  modified_base 11: mod_base = OTHER; note = 2'-O-methyladenine
  modified_base 12: mod_base = OTHER; note = locked nucleic acid base
  modified_base 13: mod_base = OTHER; note = 2'-O-methyladenine
  modified_base 14: mod_base = OTHER; note = 2'-O-methyluracil
  modified_base 15: mod_base = OTHER; note = locked nucleic acid base
  modified_base 16..17: mod_base = cm
  modified_base 18: mod_base = OTHER; note = locked nucleic acid base
  modified_base 19..20: mod_base = gm
  modified_base 21: mod_base = OTHER; note = locked nucleic acid base
  modified_base 22..23: mod_base = OTHER; note = 2'-O-methyladenine
  modified_base 24: mod_base = OTHER; note = locked nucleic acid thymine
  modified_base 25: mod_base = gm
  modified_base 26: mod_base = OTHER; note = 2'-O-methyuracil
  modified_base 27..29: mod_base = OTHER; note = locked nucleic acid base
  misc_feature 1..3: note = phosphorothioate backbone
  misc_feature 22..29: note = phosphorothioate backbone
cgaagaacggaaatccctggcaatgtgat

SEQ ID NO: 2 - RNA, 21 nt, synthetic construct
  source 1..21: mol_type = other RNA
  modified_base 1..2: mod_base = OTHER; note = locked nucleic acid thymine
  modified_base 3: mod_base = OTHER; note = 2'-O-methyladenosine
  modified_base 4: mod_base = OTHER; note = locked nucleic acid thymine
  modified_base 5..8: mod_base = OTHER; note = 2'-O-methylated base
  modified_base 9: mod_base = OTHER; note = 2'-fluoro G
  modified_base 10: mod_base = OTHER; note = 2'-O methyl U
  modified_base 11..13: mod_base = OTHER; note = 2'-fluoro A
  modified_base 14..18: mod_base = OTHER; note = 2'-O methylated base
  modified_base 19: mod_base = OTHER; note = locked nucleic acid T
  modified_base 20..21: mod_base = OTHER; note = 2'-O-methyladenosine
  misc_feature 1..3: note = phosphorothioate backbone
  misc_feature 19..21: note = phosphorothioate backbone
ttatatcagtaaagagattaa

SEQ ID NO: 3 - RNA, 11 nt, synthetic construct
  source 1..11: mol_type = other RNA
  modified_base 1..11: mod_base = OTHER; note = 2'-O methylated base
  misc_feature 11: note = The 3' terminus is attached to a C3 spacer that is joined to SEQ ID NO: 4.
tccgttcttcg

SEQ ID NO: 4 - RNA, 21 nt, synthetic construct
  source 1..21: mol_type = other RNA
  modified_base 1: mod_base = OTHER; note = 2'-O methyl U
  modified_base 2: mod_base = OTHER; note = 2'-fluoro U
  modified_base 3..5: mod_base = OTHER; note = 2'-O methylated base
  modified_base 6: mod_base = OTHER; note = locked nucleic acid C
  modified_base 7..11: mod_base = OTHER; note = 2'-O methylated base
  modified_base 12: mod_base = OTHER; note = 2'-fluoro A
  modified_base 13: mod_base = cm
  modified_base 14: mod_base = OTHER; note = 2'-fluoro U
  modified_base 15..21: mod_base = OTHER; note = 2'-O methylated base
  misc_feature 1..3: note = phosphorothioate backbone
  misc_feature 21: note = The 3' terminus is attached to a C3 spacer that is joined to SEQ ID NO: 5.
ttaatctctttactgatataa

SEQ ID NO: 5 - RNA, 11 nt, synthetic construct
  source 1..11: mol_type = other RNA
  modified_base 1..11: mod_base = OTHER; note = 2'-O-methylated base
  misc_feature 1..2: note = phosphorothioate backbone
tgccagggatt

SEQ ID NO: 6 - RNA, 11 nt, synthetic construct
  source 1..11: mol_type = other RNA
  modified_base 1..11: mod_base = OTHER; note = 2'-O methylated base
  misc_feature 11: note = The 3' terminus is attached to a C3 spacer that is joined to SEQ ID NO: 7.
tccgttcttcg

SEQ ID NO: 7 - RNA, 33 nt, synthetic construct
  source 1..33: mol_type = other RNA
  modified_base 1: mod_base = OTHER; note = 2'-O methyl U
  modified_base 2: mod_base = OTHER; note = 2'-fluoro U
  modified_base 3..5: mod_base = OTHER; note = 2'-O methylated base
  modified_base 6: mod_base = OTHER; note = 2'-fluoro C
  modified_base 7..11: mod_base = OTHER; note = 2'-O methylated base
  modified_base 12: mod_base = OTHER; note = 2'-fluoro A
  modified_base 13: mod_base = cm
  modified_base 14: mod_base = OTHER; note = 2'-fluoro U
  modified_base 15..33: mod_base = OTHER; note = 2'-O methylated base
ttaatctctttactgatataattgccagggatt

SEQ ID NO: 8 - RNA, 33 nt, synthetic construct
  source 1..33: mol_type = other RNA
  modified_base 1..13: mod_base = OTHER; note = 2'-O methylated base
  misc_feature 13..15: note = phosphorothioate backbone
  modified_base 14: mod_base = OTHER; note = 2'-fluoro U
  modified_base 15..17: mod_base = OTHER; note = 2'-O-methylated base
  modified_base 18: mod_base = OTHER; note = 2'-fluoro C
  modified_base 19..23: mod_base = OTHER; note = 2'-O methylated base
  modified_base 24: mod_base = OTHER; note = 2'-fluoro A
  modified_base 25: mod_base = cm
  modified_base 26: mod_base = OTHER; note = 2'-fluoro U
  modified_base 27..33: mod_base = OTHER; note = 2'-O methylated base
  misc_feature 33: note = The 3' terminus is attached to a C3 spacer that is joined to SEQ ID NO: 9.
tccgttcttcgattaatctctttactgatataa

SEQ ID NO: 9 - RNA, 11 nt, synthetic construct
  source 1..11: mol_type = other RNA
  modified_base 1..11: mod_base = OTHER; note = 2'-O methylated base
  misc_feature 1..2: note = phosphorothioate backbone
tgccagggatt

SEQ ID NO: 10 - RNA, 45 nt, synthetic construct
  source 1..45: mol_type = other RNA
  modified_base 1..13: mod_base = OTHER; note = 2'-O methylated base
  modified_base 14: mod_base = OTHER; note = 2'-fluoro U
  modified_base 15..17: mod_base = OTHER; note = 2'-O methylated base
  modified_base 18: mod_base = OTHER; note = 2'-fluoro C
  modified_base 19..23: mod_base = OTHER; note = 2'-O-methylated base
  modified_base 24: mod_base = OTHER; note = 2'-fluoro A
  modified_base 25: mod_base = cm
  modified_base 26: mod_base = OTHER; note = 2'-fluoro U
  modified_base 27..45: mod_base = OTHER; note = 2'-O methylated base
  misc_feature 13..15: note = phosphorothioate backbone
  misc_feature 33..35: note = phosphorothioate backbone
tccgttcttcgattaatctctttactgatataattgccagggatt

SEQ ID NO: 11 - RNA, 44 nt, synthetic construct
  source 1..44: mol_type = other RNA
  modified_base 1..12: mod_base = OTHER; note = 2'-O methyl base
  modified_base 13: mod_base = OTHER; note = 2'-fluoro U
  modified_base 14..16: mod_base = OTHER; note = 2'-O-methyl base
  modified_base 17: mod_base = OTHER; note = 2'-fluoro C
  modified_base 18..22: mod_base = OTHER; note = 2'-O-methyl base
  modified_base 23: mod_base = OTHER; note = 2'-fluoro A
  modified_base 24: mod_base = cm
  modified_base 25: mod_base = OTHER; note = 2'-fluoro U
  modified_base 26..44: mod_base = OTHER; note = 2'-O-methyl base
  misc_feature 32..34: note = phosphorothioate backbone
  misc_feature 12..14: note = phosphorothioate backbone
tccgttcttcgttaatctctttactgatataattgccagggatt

SEQ ID NO: 12 - RNA, 43 nt, synthetic construct
  source 1..43: mol_type = other RNA
  modified_base 1..12: mod_base = OTHER; note = 2'-O-methyl base
  modified_base 13: mod_base = OTHER; note = 2'-fluoro U
  modified_base 14..16: mod_base = OTHER; note = 2'-O-methyl base
  modified_base 17: mod_base = OTHER; note = 2'-fluoro C
  modified_base 18..22: mod_base = OTHER; note = 2'-O-methyl base
  modified_base 23: mod_base = OTHER; note = 2'-fluoro A
  modified_base 24: mod_base = cm
  modified_base 25: mod_base = OTHER; note = 2'-fluoro U
  modified_base 26..43: mod_base = OTHER; note = 2'-O-methyl base
  misc_feature 12..14: note = phosphorothioate backbone
  misc_feature 32..34: note = phosphorothioate backbone
tccgttcttcgttaatctctttactgatataatgccagggatt

SEQ ID NO: 13 - RNA, 21 nt, synthetic construct
  source 1..21: mol_type = other RNA
  misc_binding 1: bound_moiety = Cy3
  modified_base 1: mod_base = OTHER; note = 2'-O-methyl U
  modified_base 2: mod_base = OTHER; note = locked nucleic acid A
  modified_base 3: mod_base = cm
  modified_base 4: mod_base = OTHER; note = 2'-O-methyladenosine
  modified_base 5..6: mod_base = gm
  modified_base 7: mod_base = OTHER; note = 2'-fluoro A
  modified_base 8: mod_base = OTHER; note = 2'-O-methyladenosine
  modified_base 9..11: mod_base = OTHER; note = 2'-fluoro base
  modified_base 12..21: mod_base = OTHER; note = 2'-O-methyl base
  misc_feature 1..3: note = phosphorothioate backbone
  misc_feature 19..21: note = phosphorothioate backbone
tacaggaaaagccaaacaaca

SEQ ID NO: 14 - RNA, 31 nt, synthetic construct
  source 1..31: mol_type = other RNA
  misc_binding 1: bound_moiety = triethylene glycol spacer
  misc_binding 31: bound_moiety = Cholesterol-TEG
  modified_base 1: mod_base = cm
  modified_base 2: mod_base = OTHER; note = locked nucleic acid T
  modified_base 3: mod_base = OTHER; note = 2'-O-methyl U
  modified_base 4: mod_base = cm
  modified_base 5: mod_base = OTHER; note = locked nucleic acid A
  modified_base 6..7: mod_base = cm
  modified_base 8: mod_base = OTHER; note = locked nucleic acid A
  modified_base 9: mod_base = cm
  modified_base 10: mod_base = OTHER; note = locked nucleic acid
  modified_base 11..13: mod_base = OTHER; note = 2'-O-methyl base
  modified_base 14: mod_base = OTHER; note = locked nucleic acid base
  modified_base 15..16: mod_base = OTHER; note = 2'-O-methyl base
  modified_base 17: mod_base = OTHER; note = locked nucleic acid T
  modified_base 18: mod_base = gm
  modified_base 19: mod_base = OTHER; note = locked nucleic acid base
  modified_base 20: mod_base = cm
  modified_base 21: mod_base = OTHER; note = 2'-O-methyladenosine
  modified_base 22: mod_base = OTHER; note = locked nucleic acid base
  modified_base 23..25: mod_base = OTHER; note = 2'-O-methyl base
  modified_base 26: mod_base = OTHER; note = locked nucleic acid base
  modified_base 27: mod_base = OTHER; note = 2'-O-methyladenosine
  modified_base 28: mod_base = OTHER; note = locked nucleic acid base
  modified_base 29: mod_base = cm
  modified_base 30: mod_base = OTHER; note = locked nucleic acid base
  modified_base 31: mod_base = OTHER; note = 2'-O-methyladenosine
  misc_feature 23..31: note = phosphorothioate backbone
cttcaccacctctcagtggcaatgcgaccaa

SEQ ID NO: 15 - RNA, 33 nt, synthetic construct
  source 1..33: mol_type = other RNA
  modified_base 1..13: mod_base = OTHER; note = 2'-O-methyl base
  modified_base 14: mod_base = OTHER; note = 2'-fluoro G
  modified_base 15..17: mod_base = OTHER; note = 2'-O-methyl base
  modified_base 18: mod_base = OTHER; note = 2'-fluoro U
  modified_base 19..23: mod_base = OTHER; note = 2'-O-methyl base
  modified_base 24: mod_base = OTHER; note = 2'-fluoro U
  modified_base 25: mod_base = OTHER; note = 2'-O-methyl base
  modified_base 26: mod_base = OTHER; note = 2'-fluoro U
  modified_base 27..33: mod_base = OTHER; note = 2'-O-methyl base
  misc_feature 33: note = The 3' terminus is attached to a C3 space via a phosphorothioate backbone, which is joined to SEQ ID NO: 16.
  misc_feature 13..15: note = phosphorothioate backbone
aggtggtgaagatgttgtttggcttttcctgta

SEQ ID NO: 16 - RNA, 11 nt, synthetic construct
  source 1..11: mol_type = other RNA
  modified_base 1..11: mod_base = OTHER; note = 2'-O-methyl base
  misc_feature 1..2: note = phosphorothioate backbone
ttgccactgag

SEQ ID NO: 17 - RNA, 25 nt, synthetic construct
  source 1..25: mol_type = other RNA
  misc_binding 1: bound_moiety = C3 spacer
  modified_base 1..2: mod_base = OTHER; note = locked nucleic acid
  modified_base 11: mod_base = OTHER; note = 2'-O methyl base
  modified_base 13..14: mod_base = gm
  modified_base 15: mod_base = cm
  modified_base 17: mod_base = OTHER; note = 2'-O-methyl base
  modified_base 21..25: mod_base = OTHER; note = 2'-O-methyl base
  misc_feature 1..2: note = phosphorothioate backbone
  misc_feature 23..25: note = phosphorothioate backbone
cgagtgttgtttggcttttcctgtt

SEQ ID NO: 18 - RNA, 11 nt, synthetic construct
  source 1..11: mol_type = other RNA
  modified_base 1..11: mod_base = OTHER; note = 2'-O-methyl base
  misc_feature 11: note = The 3' terminus is attached to a C3 spacer, which is joined to SEQ ID NO: 19.
aggtggtgaag

SEQ ID NO: 19 - RNA, 23 nt, synthetic construct
  source 1..23: mol_type = other RNA
  misc_feature 23: note = The 3' terminus is attached to a C3 spacer, which is joined to SEQ ID NO: 20
  misc_feature 1..2: note = phosphorothioate backbone
  misc_feature 22..23: note = phosphorothioate backbone
  modified_base 1: mod_base = cm
  modified_base 2: mod_base = OTHER; note = locked nucleic acid base
  modified_base 3: mod_base = gm
  modified_base 23: mod_base = gm
caggaaaagccaaacaacactcg

SEQ ID NO: 20 - RNA, 12 nt, synthetic construct
  source 1..12: mol_type = other RNA
  modified_base 1..12: mod_base = OTHER; note = 2'-O-methyl base
attgccattgag

SEQ ID NO: 21 - RNA, 23 nt, synthetic construct
  source 1..23: mol_type = other RNA
  modified_base 15..23: mod_base = OTHER; note = 2'-O-methyl base
  modified_base 14: mod_base = OTHER; note = 2'-fluoro U
  modified_base 13: mod_base = cm
  modified_base 12: mod_base = OTHER; note = 2'-fluoro A
  modified_base 7..11: mod_base = OTHER; note = 2'-O-methyl base
  modified_base 6: mod_base = OTHER; note = 2'-fluoro C
  modified_base 3..5: mod_base = OTHER; note = 2'-O-methyl base
  modified_base 2: mod_base = OTHER; note = 2'-fluoro base
  modified_base 1: mod_base = OTHER; note = 2'-O-methyl base
  misc_feature 1..3: note = phosphorothioate backbone
  misc_feature 21..23: note = phosphorothioate backbone
ttaatatagtcatttctctaatt

SEQ ID NO: 22 - RNA, 21 nt, synthetic construct
  source 1..21: mol_type = other RNA
  misc_feature 1..3: note = phosphorothioate backbone
  misc_feature 19..21: note = phosphorothioate backbone
  modified_base 1..3: mod_base = OTHER; note = 2'-O-methyl base
  modified_base 4: mod_base = OTHER; note = locked nucleic acid T
  modified_base 5..8: mod_base = OTHER; note = 2'-O-methyl base
  modified_base 9: mod_base = OTHER; note = 2'-fluoro G
  modified_base 10: mod_base = OTHER; note = 2'-O-methyl U
  modified_base 11..13: mod_base = OTHER; note = 2'-fluoro A
  modified_base 14..18: mod_base = OTHER; note = 2'-O-methyl base
  modified_base 19: mod_base = OTHER; note = locked nucleic acid T
  modified_base 20..21: mod_base = OTHER; note = 2'-O-methyladenosine
ttatatcagtaaagagattaa

SEQ ID NO: 23 - RNA, 20 nt, synthetic construct
  source 1..20: mol_type = other RNA
  misc_feature 1..3: note = phosphorothioate backbone
  misc_feature 13..20: note = phosphorothioate backbone
  modified_base 1: mod_base = OTHER; note = 2'-O-methyl U
  modified_base 2: mod_base = OTHER; note = 2'-fluoro U
  modified_base 3: mod_base = OTHER; note = 2'-O-methyladenosine
  modified_base 4: mod_base = OTHER; note = 2-fluoro A
  modified_base 5: mod_base = OTHER; note = 2'-O-methyl U
  modified_base 6: mod_base = OTHER; note = 2'-fluoro base
  modified_base 7: mod_base = OTHER; note = 2'-O-methyl base
  modified_base 8: mod_base = OTHER; note = 2'-fluoro base
  modified_base 9: mod_base = OTHER; note = 2'-O-methyl base
  modified_base 10: mod_base = OTHER; note = 2'-fluoro base
  modified_base 11: mod_base = OTHER; note = 2'-O-methyl base
  modified_base 12: mod_base = OTHER; note = 2'-fluoro base
  modified_base 13: mod_base = cm
  modified_base 14: mod_base = OTHER; note = 2'-fluoro base
  modified_base 15: mod_base = gm
  modified_base 16: mod_base = OTHER; note = 2'-fluoro base
  modified_base 17: mod_base = OTHER; note = 2'-O-methyl base
  modified_base 18: mod_base = OTHER; note = 2'-fluoro base
  modified_base 19: mod_base = OTHER; note = 2'-O-methyl base
  modified_base 20: mod_base = OTHER; note = 2'-fluoro base
ttaatctctttactgatata

SEQ ID NO: 24 - RNA, 15 nt, synthetic construct
  source 1..15: mol_type = other RNA
  misc_binding 1: bound_moiety = C3 spacer
  misc_binding 15: bound_moiety = Cholesterol-TEG
  misc_feature 1..3: note = phosphorothioate backbone
  misc_feature 13..15: note = phosphorothioate backbone
  modified_base 1: mod_base = OTHER; note = 2'-fluoro base
  modified_base 2: mod_base = OTHER; note = 2'-O-methyladenosine
  modified_base 3: mod_base = OTHER; note = 2'-fluoro base
  modified_base 4: mod_base = OTHER; note = 2'-O-methyl base
  modified_base 5: mod_base = OTHER; note = 2'-fluoro base
  modified_base 6: mod_base = OTHER; note = 2'-O-methyladenosine
  modified_base 7: mod_base = OTHER; note = 2'-fluoro base
  modified_base 8: mod_base = gm
  modified_base 9: mod_base = OTHER; note = 2'-fluoro base
  modified_base 10: mod_base = gm
  modified_base 11: mod_base = OTHER; note = 2'-fluoro base
  modified_base 12: mod_base = OTHER; note = 2'-O-methyl base
  modified_base 13: mod_base = OTHER; note = 2'-fluoro base
  modified_base 14: mod_base = OTHER; note = 2'-O-methyladenosine
  modified_base 15: mod_base = OTHER; note = 2'-fluoro base
cagtaaagagattaa

SEQ ID NO: 25 - RNA, 21 nt, synthetic construct
  source 1..21: mol_type = other RNA
  misc_feature 1..2: note = phosphorothioate backbone
  misc_feature 19..21: note = phosphorothioate backbone
  misc_binding 1: bound_moiety = Cy3 dye
  modified_base 1..2: mod_base = OTHER; note = locked nucleic acid T
  modified_base 3: mod_base = OTHER; note = 2'-O-methyladenosine
  modified_base 4: mod_base = OTHER; note = locked nucleic acid T
  modified_base 5..8: mod_base = OTHER; note = 2'-O-methyl base
  modified_base 9: mod_base = OTHER; note = 2-fluoro base
  modified_base 10: mod_base = OTHER; note = 2-O-methyl base
  modified_base 11..13: mod_base = OTHER; note = 2'-fluoro base
  modified_base 14..18: mod_base = OTHER; note = 2'-O-methyl base
  modified_base 19: mod_base = OTHER; note = locked nucleic acid T
  modified_base 20..21: mod_base = OTHER; note = 2'-O-methyladenosine
ttatatcagtaaagagattaa

SEQ ID NO: 26 - RNA, 21 nt, synthetic construct
  source 1..21: mol_type = other RNA
  misc_feature 1..2: note = phosphorothioate backbone
  misc_feature 19..21: note = phosphorothioate backbone
  misc_binding 1: bound_moiety = 5' spacer 9
  modified_base 1..2: mod_base = OTHER; note = locked nucleic acid T
  modified_base 3: mod_base = OTHER; note = 2'-O-methyladenosine
  modified_base 4: mod_base = OTHER; note = locked nucleic acid T
  modified_base 5..8: mod_base = OTHER; note = 2'-O-methyl base
  modified_base 9: mod_base = OTHER; note = 2-fluoro base
  modified_base 10: mod_base = OTHER; note = 2-O-methyl base
  modified_base 11..13: mod_base = OTHER; note = 2'-fluoro base
  modified_base 14..18: mod_base = OTHER; note = 2'-O-methyl base
  modified_base 19: mod_base = OTHER; note = locked nucleic acid T
  modified_base 20..21: mod_base = OTHER; note = 2'-O-methyladenosine
ttatatcagtaaagagattaa

SEQ ID NO: 27 - RNA, 21 nt, synthetic construct
  source 1..21: mol_type = other RNA
  misc_feature 1..2: note = phosphorothioate backbone
  misc_feature 19..21: note = phosphorothioate backbone
  misc_binding 21: bound_moiety = 3' inverted dT
  modified_base 1..2: mod_base = OTHER; note = locked nucleic acid T
  modified_base 3: mod_base = OTHER; note = 2'-O-methyladenosine
  modified_base 4: mod_base = OTHER; note = locked nucleic acid T
  modified_base 5..8: mod_base = OTHER; note = 2'-O-methyl base
  modified_base 9: mod_base = OTHER; note = 2-fluoro base
  modified_base 10: mod_base = OTHER; note = 2-O-methyl base
  modified_base 11..13: mod_base = OTHER; note = 2'-fluoro base
  modified_base 14..18: mod_base = OTHER; note = 2'-O-methyl base
  modified_base 19: mod_base = OTHER; note = locked nucleic acid T
  modified_base 20..21: mod_base = OTHER; note = 2'-O-methyladenosine
ttatatcagtaaagagattaa

SEQ ID NO: 28 - RNA, 21 nt, synthetic construct
  source 1..21: mol_type = other RNA
  misc_feature 1..2: note = phosphorothioate backbone
  misc_feature 19..21: note = phosphorothioate backbone
  misc_binding 21: bound_moiety = 3' Cy3Sp
  modified_base 1..2: mod_base = OTHER; note = locked nucleic acid T
  modified_base 3: mod_base = OTHER; note = 2'-O-methyladenosine
  modified_base 4: mod_base = OTHER; note = locked nucleic acid T
  modified_base 5..8: mod_base = OTHER; note = 2'-O-methyl base
  modified_base 9: mod_base = OTHER; note = 2-fluoro base
  modified_base 10: mod_base = OTHER; note = 2-O-methyl base
  modified_base 11..13: mod_base = OTHER; note = 2'-fluoro base
  modified_base 14..18: mod_base = OTHER; note = 2'-O-methyl base
  modified_base 19: mod_base = OTHER; note = locked nucleic acid T
  modified_base 20..21: mod_base = OTHER; note = 2'-O-methyladenosine
ttatatcagtaaagagattaa

SEQ ID NO: 29 - RNA, 21 nt, synthetic construct
  source 1..21: mol_type = other RNA
  misc_feature 1..2: note = phosphorothioate backbone
  misc_feature 19..21: note = phosphorothioate backbone
  misc_binding 21: bound_moiety = 3' spacer 9
  modified_base 1..2: mod_base = OTHER; note = locked nucleic acid T
  modified_base 3: mod_base = OTHER; note = 2'-O-methyladenosine
  modified_base 4: mod_base = OTHER; note = locked nucleic acid T
  modified_base 5..8: mod_base = OTHER; note = 2'-O-methyl base
  modified_base 9: mod_base = OTHER; note = 2-fluoro base
  modified_base 10: mod_base = OTHER; note = 2-O-methyl base
  modified_base 11..13: mod_base = OTHER; note = 2'-fluoro base
  modified_base 14..18: mod_base = OTHER; note = 2'-O-methyl base
  modified_base 19: mod_base = OTHER; note = locked nucleic acid T
  modified_base 20..21: mod_base = OTHER; note = 2'-O-methyladenosine
ttatatcagtaaagagattaa

SEQ ID NO: 30 - RNA, 21 nt, synthetic construct
  source 1..21: mol_type = other RNA
  misc_feature 1..2: note = phosphorothioate backbone
  misc_feature 19..21: note = phosphorothioate backbone
  misc_binding 1: bound_moiety = Cy3 dye
  modified_base 1..2: mod_base = OTHER; note = locked nucleic acid T
  modified_base 3: mod_base = OTHER; note = 2'-O-methyladenosine
  modified_base 4: mod_base = OTHER; note = locked nucleic acid T
  modified_base 5..8: mod_base = OTHER; note = 2'-O-methyl base
  modified_base 9: mod_base = OTHER; note = 2-fluoro base
  modified_base 10: mod_base = OTHER; note = 2-O-methyl base
  modified_base 11..13: mod_base = OTHER; note = 2'-fluoro base
  modified_base 14..18: mod_base = OTHER; note = 2'-O-methyl base
  modified_base 19: mod_base = OTHER; note = locked nucleic acid T
  modified_base 20..21: mod_base = OTHER; note = 2'-O-methyladenosine
  misc_binding 21: bound_moiety = 3' inverted dT
ttatatcagtaaagagattaa

SEQ ID NO: 31 - RNA, 21 nt, synthetic construct
  source 1..21: mol_type = other RNA
  misc_feature 1..2: note = phosphorothioate backbone
  misc_feature 19..21: note = phosphorothioate backbone
  modified_base 1..2: mod_base = OTHER; note = locked nucleic acid T
  modified_base 3: mod_base = OTHER; note = 2'-O-methyladenosine
  modified_base 4: mod_base = OTHER; note = locked nucleic acid T
  modified_base 5..8: mod_base = OTHER; note = 2'-O-methyl base
  modified_base 9: mod_base = OTHER; note = 2-fluoro base
  modified_base 10: mod_base = OTHER; note = 2-O-methyl base
  modified_base 11..13: mod_base = OTHER; note = 2'-fluoro base
  modified_base 15..18: mod_base = OTHER; note = 2'-O-methyl base
  modified_base 19: mod_base = OTHER; note = locked nucleic acid T
  modified_base 20..21: mod_base = OTHER; note = 2'-O-methyladenosine
  modified_base 14: mod_base = OTHER; note = locked nucleic acid base
ttatatcagtaaagagattaa

SEQ ID NO: 32 - RNA, 21 nt, synthetic construct
  source 1..21: mol_type = other RNA
  misc_feature 1..2: note = phosphorothioate backbone
  misc_feature 19..21: note = phosphorothioate backbone
  modified_base 1..2: mod_base = OTHER; note = locked nucleic acid T
  modified_base 3: mod_base = OTHER; note = 2'-O-methyladenosine
  modified_base 4: mod_base = OTHER; note = locked nucleic acid T
  modified_base 5..8: mod_base = OTHER; note = 2'-O-methyl base
  modified_base 9: mod_base = OTHER; note = 2-fluoro base
  modified_base 10: mod_base = OTHER; note = locked nucleic acid base T
  modified_base 11..13: mod_base = OTHER; note = 2'-fluoro base
  modified_base 14..18: mod_base = OTHER; note = 2'-O-methyl base
  modified_base 19: mod_base = OTHER; note = locked nucleic acid T
  modified_base 20..21: mod_base = OTHER; note = 2'-O-methyladenosine
ttatatcagtaaagagattaa

SEQ ID NO: 33 - RNA, 24 nt, synthetic construct
  source 1..24: mol_type = other RNA
  misc_feature 1: note = The 5 terminus is attached to a spacer 9 via phosphorothioate backbone.
  misc_binding 24: bound_moiety = 3' AmMC6
  modified_base 1: mod_base = gm
  modified_base 2: mod_base = OTHER; note = locked nucleic acid base
  modified_base 3..4: mod_base = cm
  modified_base 5: mod_base = OTHER; note = locked nucleic acid base
  modified_base 6..8: mod_base = OTHER; note = 2'-O-methyl base
  modified_base 9: mod_base = OTHER; note = locked nucleic acid base T
  modified_base 10: mod_base = gm
  modified_base 11: mod_base = cm
  modified_base 12: mod_base = gm
  modified_base 13: mod_base = OTHER; note = locked nucleic acid base T
  modified_base 14..18: mod_base = OTHER; note = 2'-O-methyl base
  modified_base 19: mod_base = OTHER; note = locked nucleic acid base
  modified_base 20..21: mod_base = cm
  modified_base 22..23: mod_base = OTHER; note = locked nucleic acid base
  misc_structure 1..3: note = phosphorothioate backbone
  misc_feature 12..24: note = phosphorothioate backbone
gaccaagctgcgtgacacaccact

SEQ ID NO: 34 - RNA, 37 nt, synthetic construct
  source 1..37: mol_type = other RNA
  modified_base 16: mod_base = OTHER; note = 2'-fluoro base
  modified_base 17..19: mod_base = OTHER; note = 2'-O-methyl base
  modified_base 20: mod_base = OTHER; note = 2'-fluoro base
  modified_base 21..27: mod_base = OTHER; note = 2'-O-methyl base
  modified_base 28: mod_base = OTHER; note = 2'-fluoro base
  modified_base 29: mod_base = OTHER; note = 2'-O-methyl base
  modified_base 30: mod_base = OTHER; note = 2'-fluoro base
  modified_base 31..37: mod_base = OTHER; note = 2'-O-methyl base
  misc_feature 1..3: note = phosphorothioate backbone
  misc_feature 35..37: note = phosphorothioate backbone
  misc_feature 15..17: note = phosphorothioate backbone
  modified_base 1: mod_base = cm
  modified_base 2..15: mod_base = OTHER; note = 2'-O-methyl base
cacgcagcttggtctgttgtttggcttttcctgtaca

SEQ ID NO: 35 - RNA, 24 nt, synthetic construct
  source 1..24: mol_type = other RNA
  misc_binding 24: bound_moiety = 3' AmMC6
  modified_base 1: mod_base = gm
  modified_base 2: mod_base = OTHER; note = locked nucleic acid base
  modified_base 3..4: mod_base = cm
  modified_base 5: mod_base = OTHER; note = locked nucleic acid base
  modified_base 6..8: mod_base = OTHER; note = 2'-O-methyl base
  modified_base 9: mod_base = OTHER; note = locked nucleic acid base T
  modified_base 10: mod_base = gm
  modified_base 11: mod_base = cm
  modified_base 12: mod_base = gm
  modified_base 13: mod_base = OTHER; note = locked nucleic acid base T
  modified_base 14..18: mod_base = OTHER; note = 2'-O-methyl base
  modified_base 19: mod_base = OTHER; note = locked nucleic acid base
  modified_base 20..21: mod_base = cm
  modified_base 22..23: mod_base = OTHER; note = locked nucleic acid base
  misc_structure 1..3: note = phosphorothioate backbone
  misc_feature 12..24: note = phosphorothioate backbone
gaccaagctgcgtgacacaccact

SEQ ID NO: 36 - RNA, 37 nt, synthetic construct; identical to SEQ ID NO: 34
  source 1..37: mol_type = other RNA
  modified_base 16: mod_base = OTHER; note = 2'-fluoro base
  modified_base 17..19: mod_base = OTHER; note = 2'-O-methyl base
  modified_base 20: mod_base = OTHER; note = 2'-fluoro base
  modified_base 21..27: mod_base = OTHER; note = 2'-O-methyl base
  modified_base 28: mod_base = OTHER; note = 2'-fluoro base
  modified_base 29: mod_base = OTHER; note = 2'-O-methyl base
  modified_base 30: mod_base = OTHER; note = 2'-fluoro base
  modified_base 31..37: mod_base = OTHER; note = 2'-O-methyl base
  misc_feature 35..37: note = phosphorothioate backbone
  misc_feature 15..17: note = phosphorothioate backbone
  modified_base 1: mod_base = cm
  modified_base 2..15: mod_base = OTHER; note = 2'-O-methyl base

SEQ ID NO: 37 - RNA, 37 nt, synthetic construct; identical to SEQ ID NO: 34
  source 1..37: mol_type = other RNA
  modified_base 16: mod_base = OTHER; note = 2'-fluoro base
  modified_base 17..19: mod_base = OTHER; note = 2'-O-methyl base
  modified_base 20: mod_base = OTHER; note = 2'-fluoro base
  modified_base 21..27: mod_base = OTHER; note = 2'-O-methyl base
  modified_base 28: mod_base = OTHER; note = 2'-fluoro base
  modified_base 29: mod_base = OTHER; note = 2'-O-methyl base
  modified_base 30: mod_base = OTHER; note = 2'-fluoro base
  modified_base 31..37: mod_base = OTHER; note = 2'-O-methyl base
  misc_feature 35..37: note = phosphorothioate backbone
  misc_feature 15..17: note = phosphorothioate backbone
  modified_base 1: mod_base = cm
  modified_base 2..15: mod_base = OTHER; note = 2'-O-methyl base
  misc_feature 1: note = 5' spacer 9